SEQUENCE LISTING (85 sequences)

SEQ ID NO: 1 - protein, 50 aa, Artificial Sequence
  The sequence was designed and synthesized.
ACTAAAAAAACTTAACTTATATAATCACCTCCTAATAAATAAACTAAAAC

SEQ ID NO: 2 - protein, 50 aa, Artificial Sequence
  The sequence was designed and synthesized.
AAACCCCTTCCCCAACCTAAAACCCTAAAAATAACCTAACTAAAAAAAAC

SEQ ID NO: 3 - protein, 50 aa, Artificial Sequence
  The sequence was designed and synthesized.
AATCTATCTAAAACRTCTAACACTAAAAAACTCTTAACTTACCRTTAAAC

SEQ ID NO: 4 - protein, 50 aa, Artificial Sequence
  The sequence was designed and synthesized.
TCCTCTCTTCCTTCAACATTTCCCTATAAATTATCTATTCAATACAATAC

SEQ ID NO: 5 - protein, 50 aa, Artificial Sequence
  The sequence was designed and synthesized.
RCACTACRACCAACACAAAACTATAAATCTAACCCACRAAATTAAACRCC

SEQ ID NO: 6 - protein, 50 aa, Artificial Sequence
  The sequence was designed and synthesized.
CCAACTAAAAACAAATTCTCACTCAACACAAATAAAACCACCRCTCCAAC

SEQ ID NO: 7 - protein, 50 aa, Artificial Sequence
  The sequence was designed and synthesized.
RAACAATAATCTATCTCRAATTTCCCAAACAAAAATAAACRAACACTCCC

SEQ ID NO: 8 - protein, 50 aa, Artificial Sequence
  The sequence was designed and synthesized.
AAAAAAACCCTACRAAAACTAAAACTACTCAAAAAAAATTCRTAAAAACC

SEQ ID NO: 9 - protein, 50 aa, Artificial Sequence
  The sequence was designed and synthesized.
AAACACCTCACTCTCTAACAAACTCCTACTAACTAAAAATTATTAACATC

SEQ ID NO: 10 - protein, 50 aa, Artificial Sequence
  The sequence was designed and synthesized.
CAATTACAAAACAACTAAACAAACAAACAAAACTAAAAACTATACAAACA

SEQ ID NO: 11 - protein, 50 aa, Artificial Sequence
  The sequence was designed and synthesized.
CTTATACACATAATCTAACACACTCCAAATCACAAACRTCCCTAAACTAC

SEQ ID NO: 12 - protein, 50 aa, Artificial Sequence
  The sequence was designed and synthesized.
CCAAAAACTAAAACTAAAATAAAAAACRCCRAAATTAATAACTCCAAAAC

SEQ ID NO: 13 - protein, 50 aa, Artificial Sequence
  The sequence was designed and synthesized.
RACAAAAAAAACCAATCCCTTAACAACTATAAAATCCTATAACATAAACC

SEQ ID NO: 14 - protein, 50 aa, Artificial Sequence
  The sequence was designed and synthesized.
CCTTACCCTCTAACCCACTAAACRTAATCRACTATATTACTATAACTAAC

SEQ ID NO: 15 - protein, 50 aa, Artificial Sequence
  The sequence was designed and synthesized.
ACATCAAAAAAACTACRATAAAACRAACCTTCTAAACRCTCTAACAATCC

SEQ ID NO: 16 - protein, 50 aa, Artificial Sequence
  The sequence was designed and synthesized.
ACAATCTCTTTCAAAATAATCCTTTTTCTTCTTCCCACTACAAAAAACAC

SEQ ID NO: 17 - protein, 50 aa, Artificial Sequence
  The sequence was designed and synthesized.
CATCACCCCACATACCATATTTCAAACAAAAAATACACAACAAAAAACCA

SEQ ID NO: 18 - protein, 50 aa, Artificial Sequence
  The sequence was designed and synthesized.
RCCATTATCTATACTACAATCRTCTAATAACCAATAAAATCAACCAACCC

SEQ ID NO: 19 - protein, 50 aa, Artificial Sequence
  The sequence was designed and synthesized.
AACTACTACTACRAAAATTTATTCTCTTAACCTTTCCRCTTTACTTCCTC

SEQ ID NO: 20 - protein, 50 aa, Artificial Sequence
  The sequence was designed and synthesized.
ACRTCCCCRAAATAAATAATACAAACAAAAACCAAAATTTCTACATTAAC

SEQ ID NO: 21 - protein, 50 aa, Artificial Sequence
  The sequence was designed and synthesized.
TTACACTACAAAAAACCAACCCAAACTATAAAAATACACAAAACAACACA

SEQ ID NO: 22 - protein, 50 aa, Artificial Sequence
  The sequence was designed and synthesized.
AACAACTTTCCAAAACACTTTTCCTCCCATTATCTTCCTTAATACCTACC

SEQ ID NO: 23 - protein, 50 aa, Artificial Sequence
  The sequence was designed and synthesized.
CAATTAAAACATTAAAAACTAAAAAAACACAACTCACACAAAACTAAACA

SEQ ID NO: 24 - protein, 50 aa, Artificial Sequence
  The sequence was designed and synthesized.
AAAAAAACAACCACTTTTCCAATAAAAAATTAAACAAAAAATTAACACCC

SEQ ID NO: 25 - protein, 50 aa, Artificial Sequence
  The sequence was designed and synthesized.
TATTTTAAAACCRCCTTTACCAAACRACATTTCTATTTATATCTCTAAAC

SEQ ID NO: 26 - protein, 50 aa, Artificial Sequence
  The sequence was designed and synthesized.
ATCAAAACTATTTCTATCCTTATAACAACCCATAAAACCCCCAACTCTCC

SEQ ID NO: 27 - protein, 50 aa, Artificial Sequence
  The sequence was designed and synthesized.
ATAAAATACCCTACATCAACCCAAAATAACAAAAAAACTATCAAAAAAAC

SEQ ID NO: 28 - protein, 50 aa, Artificial Sequence
  The sequence was designed and synthesized.
CAATTACGAAACAACTAAACAAACGAACGAAACTAAAAACTATACAAACG

SEQ ID NO: 29 - protein, 50 aa, Artificial Sequence
  The sequence was designed and synthesized.
CATCGCCCCGCATACCGTATTTCAAACAAAAAATACACAACAAAAAACCG

SEQ ID NO: 30 - protein, 50 aa, Artificial Sequence
  The sequence was designed and sythesized.
TTACGCTACGAAAAACCAACCCGAACTATAAAAATACGCGAAACAACGCG

SEQ ID NO: 31 - protein, 50 aa, Artificial Sequence
  The sequence was designed and synthesized.
CAATTAAAACGTTAAAAACTAAAAAAACGCAACTCGCGCAAAACTAAACG

SEQ ID NO: 32 - protein, 50 aa, Artificial Sequence
  The sequence was designed and synthesized.
CGCCCCAGTCCATTCATCAGGAGGTGATCACACAAGTCAAGCTTCTCTAG

SEQ ID NO: 33 - protein, 50 aa, Artificial Sequence
  The sequence was designed and synthesized.
AGCCCCTTCCCCAACCTGAAGCCCTGGAGATGGCCTGGCTGGAGGGGACG

SEQ ID NO: 34 - protein, 50 aa, Artificial Sequence
  The sequence was designed and synthesized.
GTCTGTCTGGAGCGTCTGGCACTGGGGGGCTCTTAACTTGCCGTTGGGCG

SEQ ID NO: 35 - protein, 50 aa, Artificial Sequence
  The sequence was designed and synthesized.
CGCACTGCACTGAATAGACAATTCACAGGGAAATGTTGAAGGAAGAGAGG

SEQ ID NO: 36 - protein, 50 aa, Artificial Sequence
  The sequence was designed and synthesized.
CGGCGTCCAACCTCGTGGGCCAGACCTACAGCTCTGTGCTGGTCGTAGTG

SEQ ID NO: 37 - protein, 50 aa, Artificial Sequence
  The sequence was designed and synthesized.
CGCTGGAGCGGTGGCTTCATTTGTGCTGAGTGAGAACTTGTTCCCAGCTG

SEQ ID NO: 38 - protein, 50 aa, Artificial Sequence
  The sequence was designed and synthesized.
CGGGAGTGCCCGTCCATCTTTGCTTGGGAAATCCGAGACAGATTACTGTC

SEQ ID NO: 39 - protein, 50 aa, Artificial Sequence
  The sequence was designed and synthesized.
GGGAGGCCCTGCGGAAGCTGGGGCTGCTCAGGGAGAGTTCGTGAGGGCCG

SEQ ID NO: 40 - protein, 50 aa, Artificial Sequence
  The sequence was designed and synthesized.
GACACCTCACTCTCTGGCAGGCTCCTGCTGGCTGGAGATTGTTAACATCG

SEQ ID NO: 41 - protein, 50 aa, Artificial Sequence
  The sequence was designed and synthesized.
CAATTGCGAGGCAGCTGGACAAGCGGGCGGGGCTGAGGGCTGTGCAGGCG

SEQ ID NO: 42 - protein, 50 aa, Artificial Sequence
  The sequence was designed and synthesized.
TTATACACATAGTCTAGCACACTCCAAATCACAAACGTCCCTGGACTACG

SEQ ID NO: 43 - protein, 50 aa, Artificial Sequence
  The sequence was designed and synthesized.
CAGGAACTAGAGCTAGAGTAGGGGGCGCCGAGGTTGGTGGCTCCAGGGCG

SEQ ID NO: 44 - protein, 50 aa, Artificial Sequence
  The sequence was designed and synthesized.
CGGCCTATGTCACAGGATTTCACAGCTGCTAAGGGACTGGCCTCCTCTGC

SEQ ID NO: 45 - protein, 50 aa, Artificial Sequence
  The sequence was designed and synthesized.
CTTGCCCTCTGGCCCACTAAGCGTGATCGGCTGTGTTGCTGTGGCTGGCG

SEQ ID NO: 46 - protein, 50 aa, Artificial Sequence
  The sequence was designed and synthesized.
CATCAAAAGGGCTGCGGTAAGACGAGCCTTCTGGACGCTCTGACAGTCCG

SEQ ID NO: 47 - protein, 50 aa, Artificial Sequence
  The sequence was designed and synthesized.
CAGTCTCTTTCAAAATGGTCCTTTTTCTTCTTCCCACTGCAGGAGGCACG

SEQ ID NO: 48 - protein, 50 aa, Artificial Sequence
  The sequence was designed and synthesized.
CATCGCCCCGCATGCCGTATTTCAAACAAAGAATACACAACAAAGGGCCG

SEQ ID NO: 49 - protein, 50 aa, Artificial Sequence
  The sequence was designed and synthesized.
CGGGCTGGCTGACTCCACTGGCTATCAGACGACTGCAGCACAGACAATGG

SEQ ID NO: 50 - protein, 50 aa, Artificial Sequence
  The sequence was designed and synthesized.
GCTGCTGCTGCGAGAATTTGTTCTCTTGACCTTTCCGCTTTGCTTCCTCG

SEQ ID NO: 51 - protein, 50 aa, Artificial Sequence
  The sequence was designed and synthesized.
CGTCCCCGAAATGAATAATGCAGGCAGGAGCCAAGATTTCTGCATTAGCG

SEQ ID NO: 52 - protein, 50 aa, Artificial Sequence
  The sequence was designed and synthesized.
CGCGCTGTTCCGCGTATCCCCACAGTTCGGGCTGGTTTTCCGCAGCGCAA

SEQ ID NO: 53 - protein, 50 aa, Artificial Sequence
  The sequence was designed and synthesized.
CGGCAGGCATCAAGGAAGACAATGGGAGGAAAAGTGCCCTGGAAAGCTGC

SEQ ID NO: 54 - protein, 50 aa, Artificial Sequence
  The sequence was designed and synthesized.
CAGTTGAGACGTTGAGAGCTGGAGAGGCGCAGCTCGCGCAGGGCTGAGCG

SEQ ID NO: 55 - protein, 50 aa, Artificial Sequence
  The sequence was designed and synthesized.
GGAAGGCAGCCACTTTTCCAGTGAGAAGTTGAGCAAGGAGTTGGCACCCG

SEQ ID NO: 56 - protein, 50 aa, Artificial Sequence
  The sequence was designed and synthesized.
CGCTCAGAGACATAAACAGAAATGCCGTTTGGCAAAGGCGGCCCTAAAAC

SEQ ID NO: 57 - protein, 50 aa, Artificial Sequence
  The sequence was designed and synthesized.
TCAGGGCTATTTCTGTCCTTGTGGCAGCCCATGGGGCCCCCAGCTCTCCG

SEQ ID NO: 58 - protein, 50 aa, Artificial Sequence
  The sequence was designed and synthesized.
CGCCCCCTTGATAGTCTCCCTGCTACTTTGGGTTGATGCAGGGCATTTCA

SEQ ID NO: 59 - protein, 122 aa, Artificial Sequence
  The sequence was designed and synthesized.
TTCCACCGGGAACTAGAGAAGCTTGACTTGTGTGATCACCTCCTGATGAATGGACTGGGGCGGAACACAGATCCGGAGGTGGGGGCTTCTCTTCACCAAGCGCAAAGCCTACCTTCCAGAAG

SEQ ID NO: 60 - protein, 122 aa, Artificial Sequence
  The sequence was designed and synthesized.
GGAGGCCACAGGAGCCCCTTCCCCAACCTGAAGCCCTGGAGATGGCCTGGCTGGAGGGGACGACGGCCCGCAGCGGGGACCCGACTCACTTCTTCGTCCCCACCTGCCCTGGGGTCCGAAGG

SEQ ID NO: 61 - protein, 122 aa, Artificial Sequence
  The sequence was designed and synthesized.
ACGCACCCAGAGGAGACTCCTGGTCCCCTGTCCGGACCCCGCCCCGACCAGGTCCAGCCCCGCCCAACGGCAAGTTAAGAGCCCCCCAGTGCCAGACGCTCCAGACAGACTGCCACTCTTGG

SEQ ID NO: 62 - protein, 122 aa, Artificial Sequence
  The sequence was designed and synthesized.
TTGGGGTCGCCTCCTCTCTTCCTTCAACATTTCCCTGTGAATTGTCTATTCAGTGCAGTGCGAAATAACTGTTTGCTGGTGCCTCTCTCTCAGCCTGGAGGAGGATTTACTCTTTCTCTACC

SEQ ID NO: 63 - protein, 122 aa, Artificial Sequence
  The sequence was designed and synthesized.
AGAACTTCGTGCTCAAGATCCTCTTCTGCAAGCAGTCGGACCGCGGCCTCTACACCTGCACGGCGTCCAACCTCGTGGGCCAGACCTACAGCTCTGTGCTGGTCGTAGTGCGCGGTGAGCTC

SEQ ID NO: 64 - protein, 122 aa, Artificial Sequence
  The sequence was designed and synthesized.
ATCACAGGCAGCCAGCTGGGAACAAGTTCTCACTCAGCACAAATGAAGCCACCGCTCCAGCGTTCACCAATCAGGATTTATTTCCTCTGTTTCCACGTTTACCTAGAAGGGCTTTTCCCCTC

SEQ ID NO: 65 - protein, 122 aa, Artificial Sequence
  The sequence was designed and synthesized.
CAGACCCCCTCGGACAGTAATCTGTCTCGGATTTCCCAAGCAAAGATGGACGGGCACTCCCGCTTATACTGGGCTATTTTGCTTACAACTTCTGGAGTCGCTACTCTCGGTTTACTACCACC

SEQ ID NO: 66 - protein, 122 aa, Artificial Sequence
  The sequence was designed and synthesized.
AGGAGGCGCGCAGGGAGGCCCTGCGGAAGCTGGGGCTGCTCAGGGAGAGTTCGTGAGGGCCGCGCGGGCTCCAGTCCACCCCGTTTCTCCCCACCCTGAAGAGAGGGTGAAAGAGTCGCTGC

SEQ ID NO: 67 - protein, 122 aa, Artificial Sequence
  The sequence was designed and synthesized.
ACATCGCCTGGGGACACCTCACTCTCTGGCAGGCTCCTGCTGGCTGGAGATTGTTAACATCGCCAATCAGATGTTAATTGAATGCAAAGGAATAGGAAATAAATTCTGGTTCTGAAGGGGCA

SEQ ID NO: 68 - protein, 122 aa, Artificial Sequence
  The sequence was designed and synthesized.
ACTGCACCCCTCTCCACCCCATCCGAGGGCGGGCTGCGAGTTGGAGACCCTGCAGGCCCGCGCCTGCACAGCCCTCAGCCCCGCCCGCTTGTCCAGCTGCCTCGCAATTGGCTGCACCCTCT

SEQ ID NO: 69 - protein, 122 aa, Artificial Sequence
  The sequence was designed and synthesized.
CACCCATGTGACTTATACACATAGTCTAGCACACTCCAAATCACAAACGTCCCTGGACTACGCATCCGGCCGCCCACCACACCCCCACTCTGTGGGGTGGGGCATCCCATGAGGAGTCTTCT

SEQ ID NO: 70 - protein, 122 aa, Artificial Sequence
  The sequence was designed and synthesized.
CCGCGGCTCCCCCAGGAACTAGAGCTAGAGTAGGGGGCGCCGAGGTTGGTGGCTCCAGGGCGCCGGATCGGCTCCTTCGAGGGCCCACCGCGGCCCGAGACTCCTCCCCGGAAGTTTCCTCG

SEQ ID NO: 71 - protein, 122 aa, Artificial Sequence
  The sequence was designed and synthesized.
GATCCTACAGACCGCCTAGGGGGCCACAGTCGGGCAGTCCAAGGCTGCCCTGCGGCGACCCGGCCTATGTCACAGGATTTCACAGCTGCTAAGGGACTGGCCTCCTCTGCCGTCACCGACAC

SEQ ID NO: 72 - protein, 122 aa, Artificial Sequence
  The sequence was designed and synthesized.
CTCCCCCCCCGCCTTGCCCTCTGGCCCACTAAGCGTGATCGGCTGTGTTGCTGTGGCTGGCGCGCCCGTGCCGCCATCCTGCCCCTCCCCGCCTCTGCTGCCAACCCTCCCAGCGCCGTGTG

SEQ ID NO: 73 - protein, 122 aa, Artificial Sequence
  The sequence was designed and synthesized.
TTGCGTTTCTCCAGGGCGATCTCAGGCTTCCACAGGGTCTCCAGGGGACACCGCTGAGAGCGGACTGTCAGAGCGTCCAGAAGGCTCGTCTTACCGCAGCCCTTTTGATGTCTTGGTTCCCG

SEQ ID NO: 74 - protein, 122 aa, Artificial Sequence
  The sequence was designed and synthesized.
CTCCCTCACCTTCTGGAGCTTCCCCGAGTTCTCACAGTGAATAATGGAGAAAAATCCCCTCGTGCCTCCTGCAGTGGGAAGAAGAAAAAGGACCATTTTGAAAGAGACTGCCGCACTCTGCT

SEQ ID NO: 75 - protein, 122 aa, Artificial Sequence
  The sequence was designed and synthesized.
TGGCATTGGGTGCATCGCCCCGCATGCCGTATTTCAAACAAAGAATACACAACAAAGGGCCGGCCGGCGCGGGGATTTGTCAGAATTCGGCGGTGGGAGAGATTTATCTAAGCTGAGTGGAG

SEQ ID NO: 76 - protein, 122 aa, Artificial Sequence
  The sequence was designed and synthesized.
CGGGACAGAGCGCCATTGTCTGTGCTGCAGTCGTCTGATAGCCAGTGGAGTCAGCCAGCCCGGGAGGAAGGCGGAGGGTGACCGGAACTAGGCCTTGGGGCGCAGAGAGGTGGTCATCTGTC

SEQ ID NO: 77 - protein, 122 aa, Artificial Sequence
  The sequence was designed and synthesized.
TGGTATTCTCCTTCAGAAATATCCCTCTTTCCCATCTCCTTATTCCGGGTGCAGAACGAGCGAGGAAGCAAAGCGGAAAGGTCAAGAGAACAAATTCTCGCAGCAGCAGCTTGGGGAGCGCG

SEQ ID NO: 78 - protein, 122 aa, Artificial Sequence
  The sequence was designed and synthesized.
ACAGTTTAATTTCCATTATCCGACTTCGGGATCCTAGCGCCAGCCGCCGGGGCAACGTGGCGCTAATGCAGAAATCTTGGCTCCTGCCTGCATTATTCATTTCGGGGACGTGGGGATGGGAT

SEQ ID NO: 79 - protein, 122 aa, Artificial Sequence
  The sequence was designed and synthesized.
TGGGGAATTTGTTTGCGCTGCGGAAAACCAGCCCGAACTGTGGGGATACGCGGAACAGCGCGTCTGGGGCAGGGTCGGGCCTCCCTCACTTATGCTCAGCCCGAAAGGGAGGGAGGCGATGC

SEQ ID NO: 80 - protein, 122 aa, Artificial Sequence
  The sequence was designed and synthesized.
TTGAGGTAATTTTTATTTCTCATTCCGCAGGACAGGAAACAGGGACAAAGAAAAAGAGAACGGCAGGCATCAAGGAAGACAATGGGAGGAAAAGTGCCCTGGAAAGCTGCCTATTAGGAATT

SEQ ID NO: 81 - protein, 122 aa, Artificial Sequence
  The sequence was designed and synthesized.
CACCTCCCGCTTCGTCTGTGCGCTTTTCCTCTCCGCAGCCTTCCTGGGCCGCCTCACGCGCGCTCAGCCCTGCGCGAGCTGCGCCTCTCCAGCTCTCAACGTCTCAACTGCTGTTCATCGCC

SEQ ID NO: 82 - protein, 122 aa, Artificial Sequence
  The sequence was designed and synthesized.
GCTCTTCTCTACCACGCGATCCAGCTTAGCTTTTACCTTTTCTCTAGAGGTAGAGAGGGCCGGGTGCCAACTCCTTGCTCAACTTCTCACTGGAAAAGTGGCTGCCTTCCTCCGTCTCCCTC

SEQ ID NO: 83 - protein, 122 aa, Artificial Sequence
  The sequence was designed and synthesized.
GATCCCTCCACGGCTTCCCCCTTATCTAGAGCATCTGGCCCCATCTTGGCCGTCTCTGCTCGCTCAGAGACATAAACAGAAATGCCGTTTGGCAAAGGCGGCCCTAAAACATCTTTCCCTGA

SEQ ID NO: 84 - protein, 122 aa, Artificial Sequence
  The sequence was designed and synthesized.
TGCCCTCTCCCCGGGGAAATCAAAGGTCAGGACCCTCAGCACCTCCCCCGCCTGCCCTGCCGGAGAGCTGGGGGCCCCATGGGCTGCCACAAGGACAGAAATAGCCCTGACTTTCCAGGGAG

SEQ ID NO: 85 - protein, 122 aa, Artificial Sequence
  The sequence was designed and synthesized.
CAAGGAGACATTGTCCCAGGTAGGATGTGTCCCAGCAGGAAAGGACAGGCAGGTTACTTTCGCCCCCTTGATAGTCTCCCTGCTACTTTGGGTTGATGCAGGGCATTTCATGGAAGATCATG

(19) United States
(12) Patent Application Publication
(10) Pub. No.: US 2020/0270683 A1
(43) Pub. Date: Aug. 27, 2020

(54) METHODS FOR OBTAINING EMBRYONIC STEM CELL DNA METHYLATION SIGNATURES

(71) Applicants: BROWN UNIVERSITY, Providence, RI (US); THE REGENTS OF THE UNIVERSITY OF CALIFORNIA, Oakland, CA (US); THE TRUSTEES OF DARTMOUTH COLLEGE, Hanover, NH (US); UNIVERSITY OF KANSAS, Lawrence, KS (US)

(72) Inventors: Karl T. Kelsey, Brookline, MA (US); John K. Wiencke, San Francisco, CA (US); Lucas A. Salas, Lebanon, NH (US); Devin C. Koestler, Kansas City, KS (US); Brock C. Christensen, Lebanon, NH (US)

(21) Appl. No.: 16/650,761

(22) PCT Filed: Sep. 26, 2018
(86) PCT No.: PCT/US2018/052847

§ 371 Date: Mar. 25, 2020

Related U.S. Application Data
(60) Provisional application No. 62/563,354, filed on Sep. 26, 2017.

(51) Int. Cl. C12Q 1/6881

(52) U.S. Cl. CPC C12Q 1/6881; C12Q 2600/154

(57) ABSTRACT

Stem cell maturation is a fundamental, yet poorly understood aspect of human development. A DNA methylation signature deeply reminiscent of embryonal stem cells was devised to interrogate the evolving character of multiple human tissues. The cell fraction displaying the signature was found to be highly dependent upon developmental stage (fetal vs adult) and in leukocytes, it described a dynamic transition during the first 5 years of life. Significant individual variation in the embryonic signature of leukocytes was evident at birth, in childhood, and throughout adult life. The genes denoting the signature included transcription factors and proteins intimately involved in embryonic development. The DNA methylation signature traces the developmental origin of cells and informs the study of stem cell heterogeneity in humans under homeostatic and pathologic conditions.

### RELATED APPLICATION

The present application claims the benefit of provisional application Ser. No. 62/563,354 entitled “Methods and compositions for obtaining embryonic stem cell DNA methylation signatures”, filed Sep. 26, 2017 with inventors Karl T. Kelsey, John K. Wiencke, Lucas A. Salas, Devin C. Koestler, and Brock C. Christensen, which is hereby incorporated herein by reference in its entirety

### GOVERNMENT SUPPORT

This invention was made with government support under grant numbers R01CA052689, P50CA097257, R01DE022772, R01CA207110 and P20GM103418 awarded by the National Institutes of Health. The government has certain rights in the invention.

### TECHNICAL FIELD

The invention provides methods and compositions for determining embryonic stem cell DNA methylation signatures for use in diagnostics for epidemiological, prenatal, neonatal, toxicological and oncological applications.

### BACKGROUND

The sources and diversity of hematopoietic stem cells (HSC) remain controversial (Orkin S H et al. 2008. Cell 132: 631-644). Heterogeneity in HSC populations is well established (Muller-Sieburg C E et al. 2012. Blood 119: 3900-7) with hematopoiesis in fetal and early life representing dynamic periods of stem cell transition and maturation (Herzenberg L A. 2015. Ann N Y Acad Sci 1362: 1-5; Dykstra B et al. 2008. Cell Tissue Res 331: 91-101; Copley M R et al. 2013. Exp Mol Med 45: e55). In mice, potential regulators of HSC maturation include Polycomb repressor complex 2 proteins (PRC2) (Mochizuki-Kashio M et al. 2011. Blood 118: 6553-61; Xie H et al. 2014. Cell Stem Cell 14: 68-80; Oshima M et al. 2016. Exp Hematol 44: 282-96.e3), Sox17 (He S et al. 2011. Genes Dev 25: 1613-27), Arid3a (Ratliff M L et al. 2014. Front Immunol 5: 113) and Let7B microRNA (Copley M R et al. 2013. Nat Cell Biol 15: 916-25; Rowe R G et al. 2016. J Exp Med 213: 1497-512).

Direct tracking of stem cell lineage and diversity has been achieved in experimental animal models by enumerating chromosomal translocations, retroviral insertions and molecular barcodes in repopulating cells during hematopoietic reconstitution (Eaves C J. 2015. Blood 125: 2605-13). Lineage tracing studies using genetically labeled HSCs, which permits stem cell tracking without engraftment, have produced contrasting data on the relative contributions of HSCs and progenitors in steady state hematopoiesis (Sawai C M et al. 2016. Immunity 45: 597-609; Sawen P et al. 2016. Cell Rep 14: 2809-18). Because genetic lineage tracing is not feasible in humans, effective strategies for identifying and defining distinct stem cell lineages remain to be developed.

There is a need for methods and compositions to be used for conveniently obtaining a stem cell methylation signature, by which one may compare perinatal samples, including prenatal and neonatal samples, with adult samples, to facilitate ability to track stem cells and their lineages.

### BRIEF DESCRIPTION OF THE DRAWINGS

FIG. 1A and FIG. 1B are graphical representations of discovery (FIG. 1A) and replication (FIG. 1B) of the deconvolution method using lineage invariant, developmentally sensitive CpG loci in newborn and adult peripheral blood leukocytes. Estimated mean percentage (standard deviation; SD) fetal cell origin (FCO) methylation fractions are 85.4% (6.0) for umbilical cord blood and 0.6% (1.7) for peripheral adult blood in FIG. 1A, P=2.11×10⁻¹⁹¹. In the replication (FIG. 1B), estimated FCO methylation fractions are 89.9% (3.8) for umbilical cord blood and 2.0% (3.5) for peripheral adult blood, P=8.35×10⁻⁸¹.

FIG. 2 shows absolute difference between FCO estimated with one of the CpG probe lost compared to the full set of 27 CpG probes. The y axis represents the differenced in percentages, with the 27 probes arranged on the x axis.

FIG. 3 shows the Root Mean Square Error increase per CpG lost. In the x axis, 0 corresponds to the reference containing the full set of 27 CpG probes; 1, corresponds to 27 combinations losing one CpG, 2 to 351 combinations losing 2 CpGs, 3 to 2925 combinations losing 3 CpGs, 4 to 17550 combinations losing four CpGs, and 5 to 80730 combinations losing 5 CpGs.

FIG. 4 is a graphical representation of evaluation of extent of potential maternal contamination in the discovery datasets, using umbilical cord blood (UCB).

FIG. 5 is a graphical representation of evaluation of extent of potential maternal contamination in the validation datasets, using umbilical cord blood (UCB), FCO estimated proportion (Fetal.proportion).

FIG. 6 is a graphical representation of evaluation of potential maternal contamination in the five independent datasets compared to the FCO estimation, using umbilical cord blood (UCB), FCO estimated proportion (Fetal.proportion).

FIG. 7 is a flow chart illustrating the pipeline for discovery of the ESC methylation signature. The steps include Discovery datasets which are cell-specific methylation data from B cells, CD4T cells, CD8 T cells, NK cells, granulocytes and monocytes; identifying library of stem cell lineage markers which is a three-step filtering process starting with 1,255 CpGs determined to be differentially methylated between UCVB and AWB shared across the six cell types, then filtering those CpGs to obtain sites where methylation differences between UCB and AWN were consistent, then filter CpGs to those with minimal residual cell-specific effects via confirmatory principal components analysis. The proportion of cells exhibiting the stem cell lineage signature is determined using the final library of 27 CpGs, and the reliability and validity of the signature was determined using two orthogonal approaches.

FIG. 8A-FIG. 8D illustrate selection of invariant loci for the FCO signatures. FIG. 8A and FIG. 8B show data from 1,218 candidate CpG loci, with high variability between umbilical cord blood (UCB, left side) and adults peripheral blood (APB, right side), using data from each of the leukocyte cell types. FIG. 8C and FIG. 8D show data from the reduced library of 27 CpGs with increased variability between umbilical cord blood and adult peripheral blood purified cells, and reduced variability within cell types. Candidate loci (1,218 CpGs) showed a high variability between umbilical cord blood and adult peripheral blood purified cells (principal component 1, x axis). Although small relative to the UCB/APB effect, there was a statistically significant cell type effect present among these 1,218 CpGs (principal components-PC 2 and 3, y axis in the upper panel and P heatmap in the lower panel in bold the significant variables). FIG. 8C, the reduced library (27 CpGs), showed strong separation of UCB and APB samples (principal component 1, x axis), however the residual variability from cell type was attenuated (principal component 2, y axis in the upper panel, P heatmap in the lower panel). The mAge indicates DNA methylation age.

FIG. 9A and FIG. 9B each contain a graphical representation of data obtained using artificial or synthetic mixtures of fetal cells and adult cells, with the proportion of fetal cells shown on the abscissa, and the proportion of cells carrying the FCO signature on the ordinate. Linear results were obtained using either preterm or newborn blood for generating the mixtures.

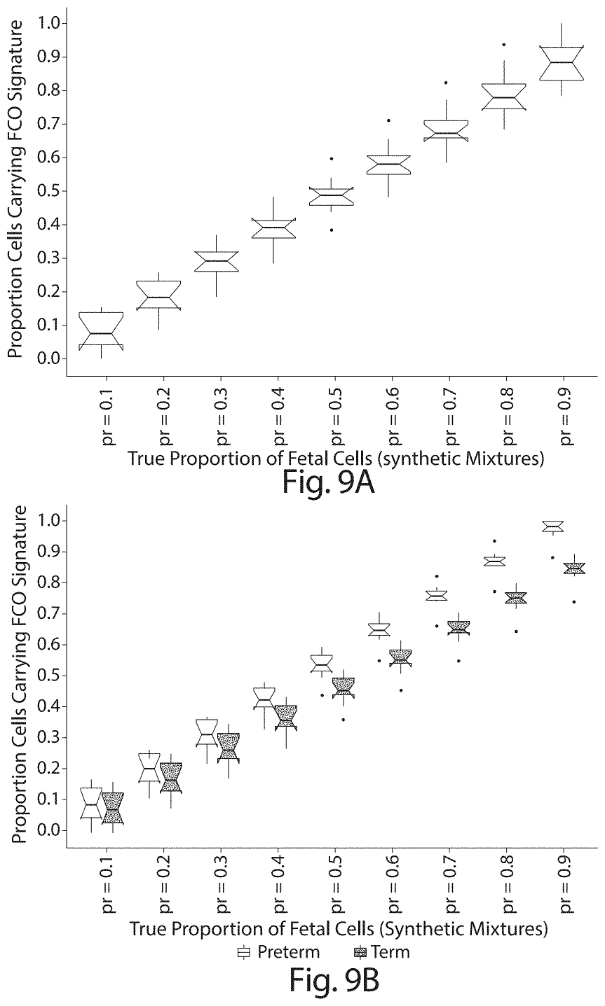

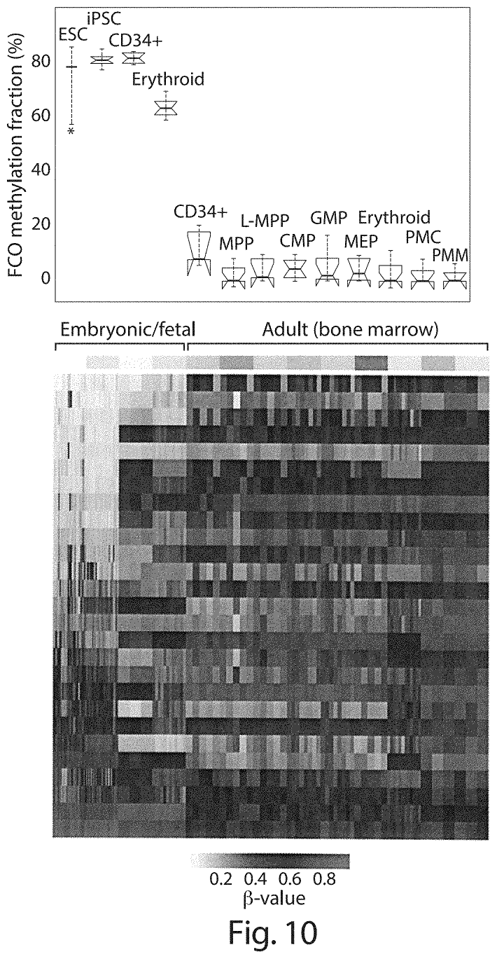

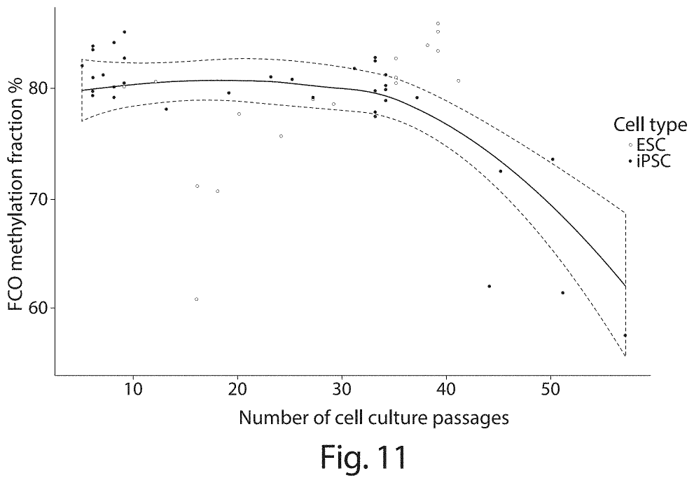

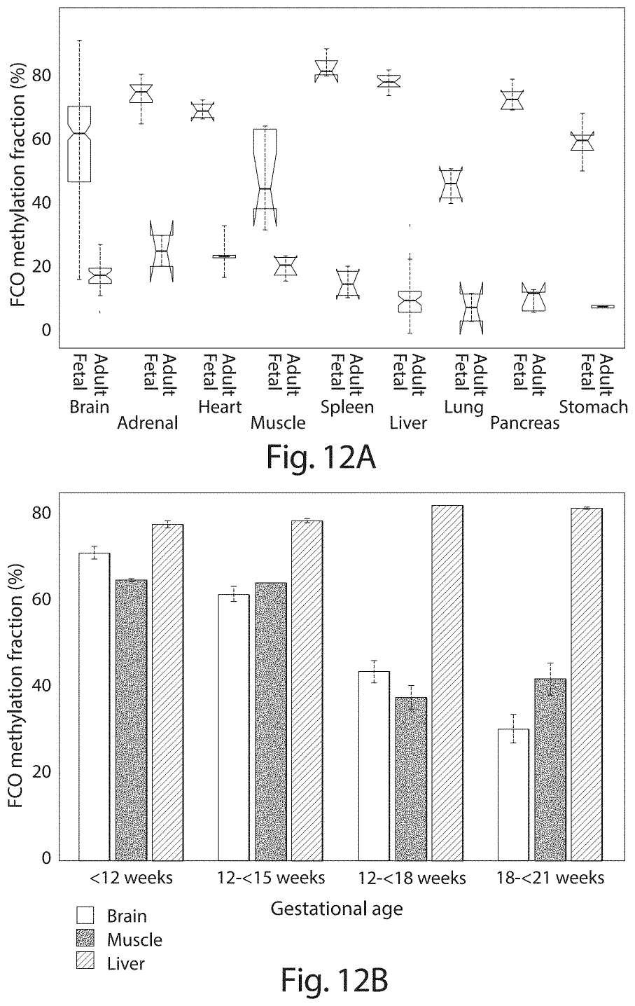

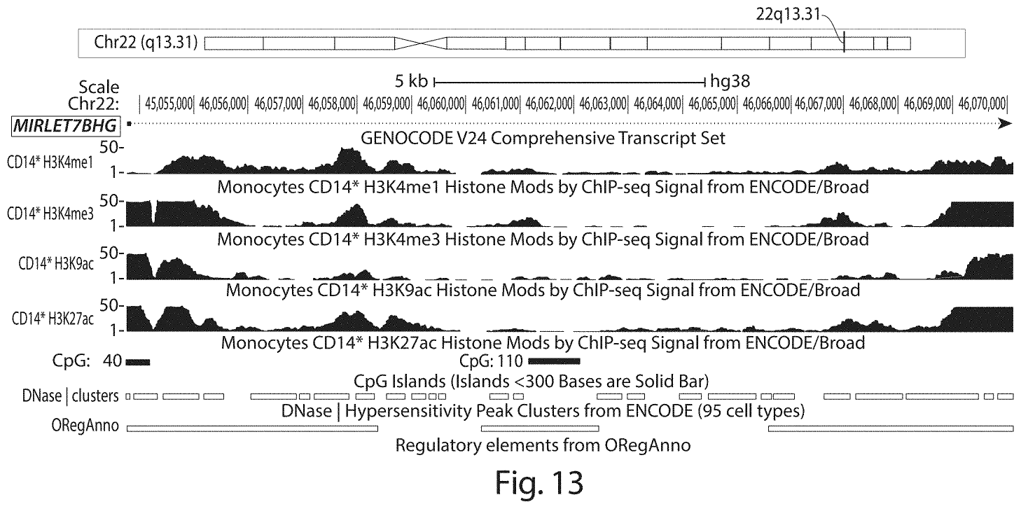

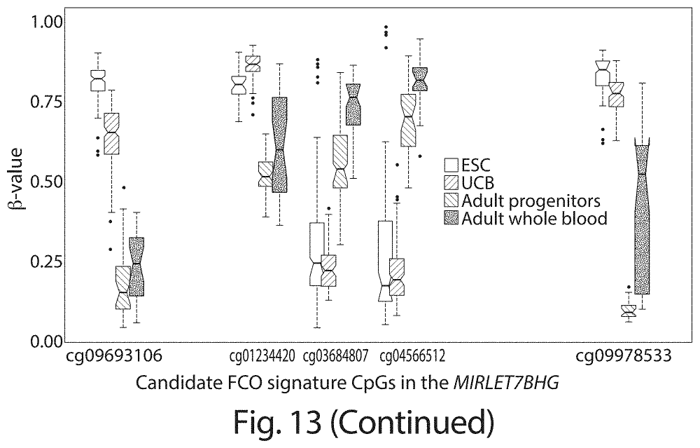

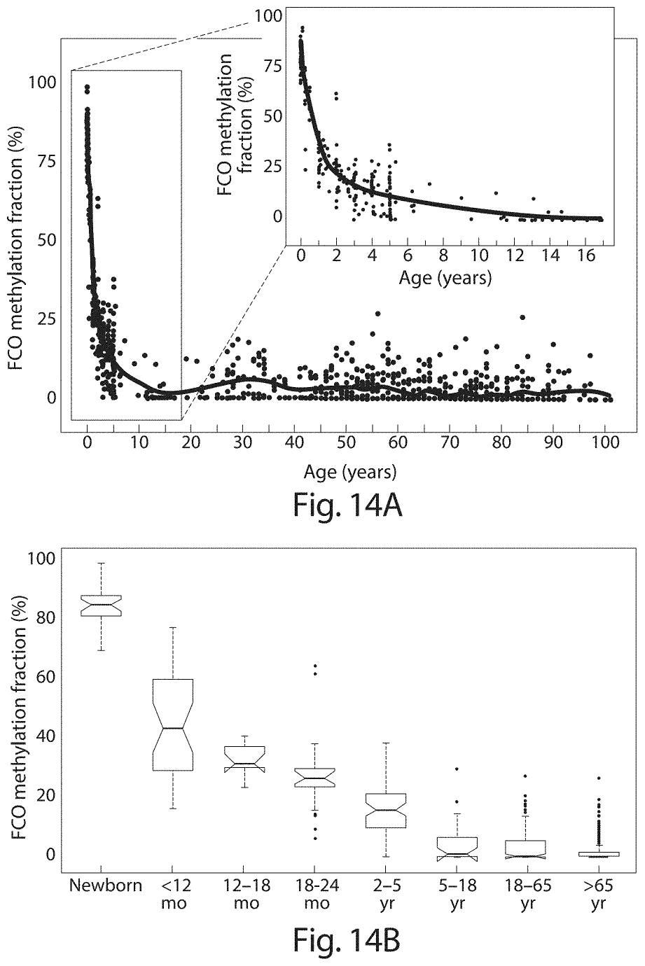

Using generated artificial synthetic mixtures, a high agreement was observed with a concordance correlation coefficient, CCC=0.97 (P<0.05). FIG. 9B includes samples from umbilical cord blood of preterms (<37 weeks of gestational age) and term newborns (≥37 weeks of gestation), and mixtures generated using these two different subgroups. The CCC for the mixtures using Preterm samples was slightly higher, CCC=0.97, compared to term newborns, CCC=0.96. Although there were differences with the largest proportions of cord blood mixtures, overall there were no statistically significant differences.

FIG. 10 shows developmentally sensitive methylation signature deconvolution in pluripotent, fetal progenitors and adult CD34⁺ stem/progenitor cells. Mean (SD) estimated FCO methylation fraction for embryonic/fetal cells is 75.9% (8.5), and for adult progenitors is 4.4% (5.1) (bone marrow), P=1.81×10⁻⁸⁶. In the boxplots in the top panel: the box shows the interquartile range (IQR), the whiskers show the inner fences (1.5×IQR out of the box), the bolded line shows the median of each set of data, and the notches-horns display the 95% confidence interval of the median. Abbreviations: embryonic stem cells (ESC), induced pluripotent stem cells (iPSC), CD34⁺ fetal (fresh cord blood cells expressing CD34⁺), erythroid fetal (fetal liver CD34⁺ cells, differentiated ex vivo to express transferrin receptor and glycophorin), CD34⁺ adult (bone marrow expressing CD34⁺ CD38⁻ CD90⁺ CD45RA⁻), multipotent progenitors (MPP), lymphoid primed multipotent progenitors (L-MPP), common myeloid progenitors (CMP), granulocyte/macrophage progenitors (GMP), megakaryocyte-erythroid progenitors (MEP), erythroid adult (adult bone marrow CD34⁺ cells, differentiated ex vivo to express transferrin receptor and glycophorin), promyelocyte/myelocyte (PMC), metamyelocyte/band-myelocyte (PMN).

FIG. 11 is a graphical representation of estimated Fetal Cell Origin (FCO) in embryonic stem cells (ESC) and induced pluripotent stem cells (iPSC) through different number of cell culture passages (cell subcultures) using loess smoothing. The number of passages ranged from 5 to 57 passages.

FIG. 12A and FIG. 12B are a box plot and a bar graph, respectively, showing FCO methylation signature deconvolution in fetal/embryonic and adult tissues. FIG. 12A compares the estimated FCO methylation fraction between fetal/embryonic and adult tissues. In the boxplot: the box shows the interquartile range (IQR), the whiskers show the inner fences (1.5×IQR out of the box), the bolded line shows the median of the data, and the notches-homs display the 95% confidence interval of the median. FIG. 12B compares the estimated mean FCO methylation signature in three fetal/embryonic tissues in four gestational periods: brain tissue and muscle tissue showed a marked reduction of the signature after the 15^{th} week of gestational age. In contrast, fetal/embryonic liver showed a persistently high level of the FCO signature.

FIG. 13 compares candidate CpGs, identified on the abscissa, in the LET7BHG locus on chromosome 22, with respect to DNA methylation levels for embryonic stems cells, umbilical cord blood, adult progenitors and adult whole blood. Patterns of methylation as a function of development were observed to depend upon the particular CpG locus. Box plots compare the DNA methylation levels (as β-values) at each CpG site for embryonic stem cells (ESC or FCO, in yellow), umbilical cord blood (UCB, in orange), adult progenitors (in green), and adult whole blood (in magenta). In the boxplots: the box shows the interquartile range (IQR), the whiskers show the inner fences (1.5×IQR out of the box), the bolded line shows the median of the data, and the notches-homs display the 95% confidence interval of the median. The scale of the boxplots was rearranged to approximate the different genomic context measured by the probes. Above the boxplots, tracks from the UCSC genome browser show the epigenomic features of normal adult CD14⁺ monocytes including activating histone marks, DNase I hypersensitivity clusters and transcriptions factor binding sites (ORegAnno: Open regulatory Annotation Database). Differences in DNA methylation between fetal cells (ESC and UCB) and adult cells (adult progenitors and adult whole blood) were statistically significant at P<2.0×10⁻¹⁶ after Bonferroni correction for all five CpG sites. Differences in DNA methylation between FCO and Adult progenitors were significant for four out of five CpGs P<5.9×10⁻⁴ after Bonferroni correction (cg03684807 was not significant P=0.26).

FIG. 14A and FIG. 14B are graphical representations of observed FCO methylation signature deconvolution in blood leukocytes sampled starting at birth extending through childhood and adult ages. FIG. 14A shows the loess smoothing curve across different ages ranging from newborn to 101 years. In the top subplot of the panel is an enlarged depiction of the marked decrease of the fraction of cells showing the FCO signature during the first 18 years of life. FIG. 14B is a box plot that summarizes the reduction of the FCO signature at different age intervals. In the boxplots: the box shows the interquartile range (IQR), the whiskers show the inner fences (1.5×IQR out of the box), the bolded line shows the median of the data, and the notches-horns display the 95% confidence interval of the median.

### SUMMARY OF EMBODIMENTS OF THE INVENTION

An aspect of the invention herein provides a method for obtaining a stem cell DNA methylation signature in a subject, the method including: identifying subsets of methylation invariant CpGs within nucleotide sequences of a plurality of leukocyte subtypes in a prenatal or neonatal sample and in an adult sample, and selecting a subset of identified CpGs containing differentially methylated regions (DMRs) between prenatal or neonate leukocyte subtypes and adult leukocyte subtypes;

determining CpGs within a resulting selected subset that are variant between the samples, and determining CpGs within the same selected subset that are invariant between leukocyte subtypes, and comparing the determined variant CpGs and the determined invariant CpGs, to select the leukocyte subtype invariant CpGs for inclusion in a subset list; and,

preparing a stem cell methylation signature by statistically removing CpGs from the subset list based on inconsistent sign in the model beta coefficient estimates compared to the absolute mean difference between the compared groups (delta beta), and selecting the leukocyte subtype invariant CpGs with a statistical difference in methylation between the adult and prenatal or neonate samples which is greater than a pre-determined threshold, to obtain the stem cell methylation signature.

The phrase, “leukocyte subtypes” as used herein and in the claims shall mean any or at least one of leukocyte types of cells which include but are not limited to granulocytes, neutrophils, monocytes, eosinophils and lymphocytes subclasses.

The phrase, “CpG subsets” shall mean a list of sites in the genome having the dinucleotide sequence of CG, the lists indicating the location (chromosome and specific site) which can be distinguished from a second or further list, by virtue of methylation status fraction.

In an embodiment of this method, the step of preparing further includes deconvoluting a prenatal sample methylation fraction or neonate sample methylation fraction compared to all adult sample methylation fraction using constrained projection quadratic programming (CP/QP), the stem cell methylation signature being substituted for a default reference methylation library.

A further embodiment of the method includes enriching the stem cell methylation signature by applying a hypergeometric test to the stem cell methylation signature that reduces the stem cell methylation signature to CpG sequences providing maximum differences in methylation status between the prenatal or neonate sample and the adult sample by a confirmatory principal component analysis with a first component and at least one second component. For example, the first component determines the CpGs that are variant in methylation status between the prenatal sample or the neonate sample and the adult sample by using a pairwise linear model and second components determine the CpGs that are invariant in methylation status among leukocyte subtypes using a linear mixed effect model adjusted using limma to account for subject differences. For example, this embodiment may further involve using the confirmatory principal component analysis first component to account for differences in the adult sample compared to the prenatal or the neonate sample, and the second component to account for subject variability and residual cell subtype confounding.

A particular embodiment of this method further includes calculating the geometric angle between the first component (x) and the second component (y). The geometric angle calculation uses x and y as the legs of the triangle and then using the inverse trigonometric function arctangent (a tan) the geometric angle is obtained as degrees=a tan(x/y)*(180/r) with a known distribution between −90 and +90. Another particular embodiment of this method further includes selecting CpGs with maximum orthogonality of the calculated geometric angle (those closer to zero degrees) for inclusion in the stem cell methylation signature.

Another embodiment of the method further includes calculating the constrained projection quadratic programming (CP/QP) according to the equation: arg min_{w}∥Y−wM^{T}∥², such that M is the list of CpGs, w is an estimate of a fraction of cells carrying the stem cell lineage signature, and Y is based on the constrained projection quadratic programming (CP/QP).

Yet another embodiment of the method further includes validating the stem cell signature by geometrically comparing DNA methylation profiles of purified leukocyte cell subtypes, by obtaining the profiles from at least one methylation library, to DNA methylation profiles of the stem cell methylation signature.

Another embodiment of the method further includes validating the stem cell signature by geometrically comparing DNA methylation profiles of synthetic cell mixtures containing known proportions of the prenatal sample or the neonate sample and the adult sample to a DNA methylation profile of the stem cell methylation signature. The phrase, “synthetic cell mixtures” as used herein refers to cells obtained by statistically mixing data derived from samples with known phenotype characteristics, which are reference samples with a known characteristic of interest, and controls.

Another embodiment of the method further includes pooling the methylation datasets of the at least one prenatal or neonatal sample and the at least one adult sample to combine at least one methylation data subset for a specified subset of leukocyte subtypes. The phrase, “specified subset of leukocyte subtypes” as used herein means a synthetic mixture of two or more leukocyte subtypes.

Another embodiment of the method further includes adjusting mathematically the methylation datasets of the at least one prenatal sample or neonate sample and the at least one adult sample to account for at least one variable of the subject from which the samples were obtained. For example, the variables are selected from one or more of the group of: sex, DNA methylation age, and subject indicators.

Another embodiment of the method further includes implementing by the hypergeometric test the methylation reference databases to restrict the background to genes interrogated in a methylation array, and applying statistical methods to the methylation data to account for array bias.

An embodiment of the method further includes using the confirmatory principal component analysis first component to account for differences in the adult sample compared to the prenatal or the neonate sample, and the second component to account for subject variability and residual cell subtype confounding.

The method further involves, in general, the stem cell methylation signature obtained by analyzing at least one or a plurality of sequences selected from the group of: cg10338787 (SEQ ID No: 68), cg22497969b(SEQ ID No: 83), cg11968804 (SEQ ID No: 71), cg10237252 (SEQ ID No: 67), cg17310258 (SEQ ID No: 78), cg13485366 (SEQ ID No: 72), cg03455765 (SEQ ID No: 62), cg04193160 (SEQ ID No: 63), cg27367526 (SEQ ID No: 85), cg03384000 (SEQ ID No: 61), cg15575683 (SEQ ID No: 76), cg17471939 (SEQ ID No: 79), cg11199014 (SEQ ID No: 70), cg13948430 (SEQ ID No: 73), cg01567783 (SEQ ID No: 60), cg01278041 (SEQ ID No: 59), cg19005955 (SEQ ID No: 80), cg16154155 (SEQ ID No: 77), cg14652587 (SEQ ID No: 75), cg19659741 (SEQ ID No: 81), cg06705930 (SEQ ID No: 65), cg23009780 (SEQ ID No: 84), cg22130008 (SEQ ID No: 82), cg05840541 (SEQ ID No: 64), cg06953130 (SEQ ID No: 66), cg11194994 (SEQ ID No: 69), and cg14375747 (SEQ ID No: 74). The nucleotide sequences, chromosome location, the starting and ending positions according to each of builds hg 19 and hg38 are shown in Table 1, as are the SEQ ID numbers. The invention provides this set of sequences (SEQ ID NOs: 1-85 shown in Table 1), which, while they are known sequences, had not previously been grouped as a subset useful together for obtaining hemopoietic stem cell methylation signatures.

In an embodiment of this method each of the plurality of sequences include a portion of cg10338787 (SEQ ID No: 68), cg22497969 (SEQ ID No: 83), cg11968804 (SEQ ID No: 71), cg10237252 (SEQ ID No: 67), cg17310258 (SEQ ID No: 78), cg13485366 (SEQ ID No: 72), cg03455765 (SEQ ID No: 62), cg04193160 (SEQ ID No: 63), cg27367526 (SEQ ID No: 85), cg03384000 (SEQ ID No: 61), cg15575683 (SEQ ID No: 76), cg17471939 (SEQ ID No: 79), cg11199014 (SEQ ID No: 70), cg13948430 (SEQ ID No: 73), cg01567783 (SEQ ID No: 60), cg01278041 (SEQ ID No: 59), cg19005955 (SEQ ID No: 80), cg16154155 (SEQ ID No: 77), cg14652587 (SEQ ID No: 75), cg19659741 (SEQ ID No: 81), cg06705930 (SEQ ID No: 65), cg23009780 (SEQ ID No: 84), cg22130008 (SEQ ID No: 82), cg05840541 (SEQ ID No: 64), cg06953130 (SEQ ID No: 66), cg11194994 (SEQ ID No: 69), and cg14375747 (SEQ ID No: 74). In an embodiment of this method the portion includes at least one hypermethylatable CpG.

The method uses for example the prenatal or neonatal sample which is a cell or a tissue obtained from at least one of the group consisting of: a fetus, an umbilical cord, umbilical blood, an infant, a uterus, a vein, an artery, a tumor, an abnormal growth, bone marrow, a transplanted or a re-sectioned biological material, an embryo, and a cell from an embryo.

An aspect of the invention herein provides uses of the methods described herein for selecting a small number of nucleotide sequences for a custom array for efficient and economical determination of at least one of embryonic cell content, stem cell content, experiential exposure on stem cell maturation, and identity of progenitor cell lineages.

An aspect of the invention herein provides a method for determining effects of experiential exposure on stem cell maturation in a subject, the method including:

obtaining an exposure sample and a control sample from the subject and analyzing extent of hybridization of each DNA sample to each of a plurality of oligonucleotide probes attached to at least one array, the probes affixed to at least one surface and containing each of methylated CpG containing oligonucleotide sequences and unmethylated CpG containing oligonucleotide sequences and otherwise identical in nucleotide sequence, the plurality of the nucleotide sequences selected from at least one of the group of cg10338787 (SEQ ID No: 68), cg22497969 (SEQ ID No: 83), cg11968804 (SEQ ID No: 71), cg10237252 (SEQ ID No: 67), cg17310258 (SEQ ID No: 78), cg13485366 (SEQ ID No: 72), cg03455765 (SEQ ID No: 62), cg04193160 (SEQ ID No: 63), cg27367526 (SEQ ID No: 85), cg03384000 (SEQ ID No: 61), cg15575683 (SEQ ID No: 76), cg17471939 (SEQ ID No: 79), cg11199014 (SEQ ID No: 70), cg13948430 (SEQ ID No: 73), cg01567783 (SEQ ID No: 60), cg01278041 (SEQ ID No: 59), cg19005955 (SEQ ID No: 80), cg16154155 (SEQ ID No: 77), cg14652587 (SEQ ID No: 75), cg19659741 (SEQ ID No: 81), cg06705930 (SEQ ID No: 65), cg23009780 (SEQ ID No: 84), cg22130008 (SEQ ID No: 82), cg05840541 (SEQ ID No: 64), cg06953130 (SEQ ID No: 66), cg11194994 (SEQ ID No: 69), and cg14375747 (SEQ ID No: 74), and determining a methylation status of at least one CpG dinucleotide in the DNA of the exposure sample and a methylation status of at least one CpG dinucleotide in the DNA of the control sample; and,

deconvoluting the methylation array data from the control sample and the exposure sample to obtain methylation status of individual leukocyte subtypes in the samples, and comparing methylation status of the at least one CpG dinucleotide within a leukocyte subtype of the control sample to the methylation status of the at least one CpG dinucleotide within the same leukocyte subtype of the exposure sample, to determine sites of differential methylation, and correlating a difference in methylation status between the control sample and the exposure sample to obtain the effect of the exposure on stem cell methylation signature.

An aspect of the invention herein provides a method for determining effects of experiential exposure on stem cell maturation in a subject, the method including:

obtaining an exposure sample and a control sample from the subject and analyzing extent of methylation of at least one CpG dinucleotide in DNA of each sample within a plurality of oligonucleotides sequences selected from at least one of the group of cg10338787 (SEQ ID No: 68), cg22497969 (SEQ ID No: 83), cg11968804 (SEQ ID No: 71), cg10237252 (SEQ ID No: 67), cg17310258 (SEQ ID No: 78), cg13485366 (SEQ ID No: 72), cg03455765 (SEQ ID No: 62), cg04193160 (SEQ ID No: 63), cg27367526 (SEQ ID No: 85), cg03384000 (SEQ ID No: 61), cg15575683 (SEQ ID No: 76), cg17471939 (SEQ ID No: 79), cg11199014 (SEQ ID No: 70), cg13948430 (SEQ ID No: 73), cg01567783 (SEQ ID No: 60), cg01278041 (SEQ ID No: 59), cg19005955 (SEQ ID No: 80), cg16154155 (SEQ ID No: 77), cg14652587 (SEQ ID No: 75), cg19659741 (SEQ ID No: 81), cg06705930 (SEQ ID No: 65), cg23009780 (SEQ ID No: 84), cg22130008 (SEQ ID No: 82), cg05840541 (SEQ ID No: 64), cg06953130 (SEQ ID No: 66), cg11194994 (SEQ ID No: 69), and cg14375747 (SEQ ID No: 74), thereby determining a methylation status of at least one CpG dinucleotide in the DNA of the exposure sample and a methylation status of at least one CpG dinucleotide in the DNA of the control sample; and,

deconvoluting the methylation array data from the control sample and the exposure sample to obtain methylation status of individual leukocyte subtypes in the samples, and comparing methylation status of the at least one CpG dinucleotide within a leukocyte subtype of the control sample to the methylation status of the at least one CpG dinucleotide within the same leukocyte subtype of the exposure sample, to determine sites of differential methylation, and correlating a difference in methylation status between the control sample and the exposure sample to obtain the effect of the exposure on stem cell methylation signature.

In embodiments of this method extent of methylation is determined by hybridizing each DNA sample to each of a plurality of oligonucleotide probes attached to at least one array, the probes affixed to at least one surface and containing each of methylated CpG containing oligonucleotide sequences and unmethylated CpG containing oligonucleotide sequences and otherwise identical in nucleotide sequence. In an embodiment of this method extent of methylation is determined by amplifying sample DNA by polymerase chain reaction (PCR) with primers specific for hypermethylated Cpg dinucleotides.

In an embodiment of this method, correlating further involves assessing the effects of at least one of the following on the stem cell methylation signature: a therapy, a vaccine, a nutritional regimen, a genetic alteration, a progenitor cell transplant, and an environmental exposure. In an alternative embodiment of this method, correlating further involves diagnosing prenatal abnormalities in a fetus. In another alternative embodiment after correlating, the method further involves altering patient therapies through analysis of stem cell methylation in induced pluripotent stem cells therapies in the subject. In yet another alternative embodiment after correlating, the method further involves determining amount of induction of stem cell progenitors in a transplantation procedure. In yet another alternative embodiment after correlating, the method further involves measuring an extent of reprogramming adult cells into induced pluripotent stem cells, obtaining a quality control parameter.

An aspect of the invention provides a kit for determining embryonic stem cell methylation signatures, including:

an array with a plurality of DNA probes attached to a surface or a plurality of surfaces at known addressable locations on the array, such that the probes hybridize to a DNA sequence of each of a methylated form and an unmethylated form of a CpG dinucleotide in a sequence of a gene of the plurality of genes in the sample;

primers and reagents for detecting the hybridized probes and for detecting the reaction products derived from the hybridized probes to obtain methylation data; and

instructions for analyzing at least one sample on the array, and instructions for preparing a stem cell methylation signature.

In an embodiment of this kit each of the plurality of sequences include a portion of cg10338787 (SEQ ID No: 68), cg22497969 (SEQ ID No: 83), cg11968804 (SEQ ID No: 71), cg10237252 (SEQ ID No: 67), cg17310258 (SEQ ID No: 78), cg13485366 (SEQ ID No: 72), cg03455765 (SEQ ID No: 62), cg04193160 (SEQ ID No: 63), cg27367526 (SEQ ID No: 85), cg03384000 (SEQ ID No: 61), cg15575683 (SEQ ID No: 76), cg17471939 (SEQ ID No: 79), cg11199014 (SEQ ID No: 70), cg13948430 (SEQ ID No: 73), cg01567783 (SEQ ID No: 60), cg01278041 (SEQ ID No: 59), cg19005955 (SEQ ID No: 80), cg16154155 (SEQ ID No: 77), cg14652587 (SEQ ID No: 75), cg19659741 (SEQ ID No: 81), cg06705930 (SEQ ID No: 65), cg23009780 (SEQ ID No: 84), cg22130008 (SEQ ID No: 82), cg05840541 (SEQ ID No: 64), cg06953130 (SEQ ID No: 66), cg11194994 (SEQ ID No: 69), and cg14375747 (SEQ ID No: 74). In and embodiment of this kit the portion includes at least one hypermethylatable CpG.

An aspect of the invention provides a method for identifying progenitor cell lineages, the method including the following steps:

comparing DNA methylation profiles of a leukocyte subtype between a prenatal or neonatal sample and an adult sample;

identifying CpG sites differentially methylated between the prenatal or neonatal sample and the adult sample for the leukocyte subtype;

filtering to select a lineage invariant subset of CpG loci, the subset loci having consistent differential methylation between the leukocyte subtype and an absolute change in methylation greater than a pre-determined threshold between the prenatal or neonatal sample and the adult sample, thereby forming a candidate list of CpG loci for a stem cell methylation signature; and

reducing the candidate list of CpG loci for the stem cell methylation signature by selecting CpGs with minimal residual cell-specific effects, thereby forming a block of differentially methylated regions (DMRs) across the progenitor cell axis of multipotency to terminal differentiation, to identify the progenitor cell lineages. An embodiment of this method further includes calculating a leukocyte proportion exhibiting the stem cell methylation signature, by applying constrained projection quadratic programming (CP/QP) to the candidate list of the stem cell methylation signature CpG loci. For example, calculating further includes iterating with at least one additional set of leukocyte sequences from each of the prenatal or neonatal sample and the adult sample sources to confirm the candidate list of the CpG loci for the stem cell methylation signature as an estimator of the fraction of the leukocytes in a mixture that contains lineage invariant and developmentally sensitive stem cell loci. An embodiment of this method further includes: validating the calculated stem cell methylation signatures by preparing mixtures of the prenatal or neonate sample and the adult sample in known relative amounts, thereby generating synthetic cell mixtures; analyzing the synthetic cell mixtures on a DNA methylation array to determine methylation status of CpG dinucleotides in the leukocytes in the mixtures; and applying statistical methods to the obtained methylation array data of the mixtures to correlate the fraction of cells carrying a stem cell methylation signature with the known mixture relative amounts, thereby determining stem cell maturation by the changes in methylation status between the prenatal or neonate sample leukocytes and the adult sample leukocytes.

An aspect of the invention herein provides a method of using an array to determine an embryonic stem cell (ESC) methylation signature in a biological sample, including:

analyzing extent of DNA hybridization in an adult sample and a prenatal or neonatal sample to each of a plurality of oligonucleotide probes, the probes being affixed to at least a first surface for methylated CPG sequences and a second surface for unmethylated CpG sequences, the DNA sequences of the oligonucleotides on the first surface and the second surface being otherwise identical, the plurality of the nucleotide sequences selected from at least one of the group of cg10338787 (SEQ ID No: 68), cg22497969 (SEQ ID No: 83), cg11968804 (SEQ ID No: 71), cg10237252 (SEQ ID No: 67), cg17310258 (SEQ ID No: 78), cg13485366 (SEQ ID No: 72), cg03455765 (SEQ ID No: 62), cg04193160 (SEQ ID No: 63), cg27367526 (SEQ ID No: 85), cg03384000, cg15575683 (SEQ ID No: 76), cg17471939 (SEQ ID No: 79), cg11199014 (SEQ ID No: 70), cg13948430 (SEQ ID No: 73), cg01567783 (SEQ ID No: 60), cg01278041 (SEQ ID No: 59), cg19005955 (SEQ ID No: 80), cg16154155 (SEQ ID No: 77), cg14652587 (SEQ ID No: 75), cg19659741 (SEQ ID No: 81), cg06705930 (SEQ ID No: 65), cg23009780 (SEQ ID No: 84), cg22130008 (SEQ ID No: 82), cg05840541 (SEQ ID No: 64), cg06953130 (SEQ ID No: 66), cg11194994 (SEQ ID No: 69), and cg14375747 (SEQ ID No: 74), for determining methylation status of at least one CpG dinucleotide in the DNA of each of the adult and the prenatal or neonatal sample sample;

deconvoluting the methylation array data from the adult sample and the prenatal or neonatal sample to obtain methylation data of a plurality of leukocyte subtypes in the samples;

comparing methylation status of the at least one CpG dinucleotide for a leukocyte subtype in the adult sample to the methylation status of the at least one CpG dinucleotide of the leukocyte subtype of the prenatal or neonatal sample, to determine differentially methylated regions (DMRs); and

analyzing the DMRs to determine the fraction of sequences from progenitor cell lineage origin which constitutes the ESC methylation signature.

An embodiment of this method further includes comparing the ESC methylation signature of samples of a first subject and a second subject, such that the first and second subjects are assessed for effects on the embryonic stem cell methylation signature of differences in maternal or prenatal conditions selected from the group of: nutrition, nutrition, genetics, infant or embryonic genetics, environmental exposure, hematopoietic stress, treatment with chemical agents, vaccination status, transplantation, and surgical stress.

Another embodiment of this method further includes comparing the ESC methylation signature during cancer therapy induced neutropenia in a sample from a patient being treated with an agent that promote granulopoiesis, with the ESC methylation signature obtained prior to treatment.

Another embodiment of this method further includes inducing CD34 stem progenitors for transplantation, and comparing effect on the ESC methylation signatures to determine quality of the induction process. The terms ESC and FCO, fetal cell origin, as used herein refer to the same biological samples.

An aspect of the invention provides an array for efficient and economical determination of embryonic stem cell (ESC) content in a biological sample, the array having a surface containing a plurality of nucleotide sequences, each sequence at an addressable location, the sequences selected from at least one of the group of: cg10338787 (SEQ ID No: 68), cg22497969 (SEQ ID No: 83), cg11968804 (SEQ ID No: 71), cg10237252 (SEQ ID No: 67), cg17310258 (SEQ ID No: 78), cg13485366 (SEQ ID No: 72), cg03455765 (SEQ ID No: 62), cg04193160 (SEQ ID No: 63), cg27367526 (SEQ ID No: 85), cg03384000 (SEQ ID No: 61), cg15575683 (SEQ ID No: 76), cg17471939 (SEQ ID No: 79), cg11199014 (SEQ ID No: 70), cg13948430 (SEQ ID No: 73), cg01567783 (SEQ ID No: 60), cg01278041 (SEQ ID No: 59), cg19005955 (SEQ ID No: 80), cg16154155 (SEQ ID No: 77), cg14652587 (SEQ ID No: 75), cg19659741 (SEQ ID No: 81), cg06705930 (SEQ ID No: 65), cg23009780 (SEQ ID No: 84), cg22130008 (SEQ ID No: 82), cg05840541 (SEQ ID No: 64), cg06953130 (SEQ ID No: 66), cg11194994 (SEQ ID No: 69), and cg14375747 (SEQ ID No: 74), for analyzing a fraction of sequences of progenitor cell lineage origin having an ESC methylation signature.

Thus the array so customized is efficient and economical for determination of the ESC cell content, because the array contains nucleotide sequences containing CpG sites which are substantially reduced in number, such that the number of sequences is less than 1%, less than 0.1%, 0.01% or 0.001% of total CpG sequences that could be found in a genome, such as a mammalian genome, specifically, the human genome. The array having a small number of sequences provides quicker and easier analysis of ESC cell content (or FCO cell content, and is a platform from a variety of applications for diagnosis and prognosis are obtained.

For example, an array having only the 27 nucleotide sequences is used for determining any of embryonic cell content, stem cell content, results of experiential exposure on stem cell maturation, and identity of progenitor cell lineages. Thus the array having nucleotide sequences containing at least one CpG selected by any of the methods herein from among 25 million CpGs in the human genome, or preferably a plurality or all of the only 27 sequences, in a variety of applications.

The invention in various aspects provides uses of the sequences identified herein which are a small number of nucleotide sequences for obtaining a custom array, used for efficient and economical determination of at least one of embryonic cell content, stem cell content, experiential exposure on stem cell maturation, and identity of progenitor cell lineages.

An aspect of the invention herein provides a kit for determining embryonic cell content, the kit including a plurality of primers for custom bisulfate sequencing library preparation, each primer directing amplification of a hyper methylatable CpG dinucleotide located in a DNA sequence selected from cg10338787 (SEQ ID No: 68), cg22497969 (SEQ ID No: 83), cg11968804 (SEQ ID No: 71), cg10237252 (SEQ ID No: 67), cg17310258 (SEQ ID No: 78), cg13485366 (SEQ ID No: 72), cg03455765 (SEQ ID No: 62), cg04193160 (SEQ ID No: 63), cg27367526 (SEQ ID No: 85), cg03384000 (SEQ ID No: 61), cg15575683 (SEQ ID No: 76), cg17471939 (SEQ ID No: 79), cg11199014 (SEQ ID No: 70), cg13948430 (SEQ ID No: 73), cg01567783 (SEQ ID No: 60), cg01278041 (SEQ ID No: 59), cg19005955 (SEQ ID No: 80), cg16154155 (SEQ ID No: 77), cg14652587 (SEQ ID No: 75), cg19659741 (SEQ ID No: 81), cg06705930 (SEQ ID No: 65), cg23009780 (SEQ ID No: 84), cg22130008 (SEQ ID No: 82), cg05840541 (SEQ ID No: 64), cg06953130 (SEQ ID No: 66), cg11194994 (SEQ ID No: 69), and cg14375747 (SEQ ID No: 74).

An aspect of the invention herein provides a kit for determining embryonic stem cell methylation signatures, including:

an array with a plurality of DNA probes attached to a surface or a plurality of surfaces at known addressable locations on the array, such that the probes hybridize to a DNA sequence of each of a methylated form and an unmethylated form of a CpG dinucleotide in a sequence of a gene of the plurality of genes in the sample or; a set of oligonucleotide primers including a plurality of sequences each having a CpG dinucleotide within each primer sequence;

primers and reagents for detecting the hybridized probes and for detecting the reaction products derived from the hybridized probes to obtain methylation data; and

instructions for analyzing at least one sample on the array, and instructions for preparing a stem cell methylation signature.

An aspect of the invention herein provides a kit for quantifying embryonic stem cells in a biological sample, the kit including:

at least one of
(i) an array with a plurality of DNA probes attached to a surface or a plurality of surfaces at known addressable locations on the array, such that the probes hybridize to a DNA sequence of each of a methylated form and an unmethylated form of a CpG dinucleotide in a stem cell signature sequence in the sample; and/or
(ii) a plurality of oligonucleotide primers including a plurality of gene sequences in the stem cell signature for amplification of genomic DNA at a plurality of loci corresponding to hypermethylated CpG sites; and

reagents including at least one of: primers for amplifying DNA in the sample, for detecting sample DNA hybridized with probes, and for detecting reaction products derived from the hybridized probes to obtain methylation data; and

instructions for analyzing at least one sample on the array, and instructions for quantifying embryonic stem cells based on the stem cell methylation signature.

The invention in various aspects provides uses of a list of 27 CpG listed herein containing loci in the human genome as a stem cell methylation signature for efficient and economical determination of at least one of embryonic cell content, stem cell content, experiential exposure on stem cell maturation, and identity of progenitor cell lineages.

An aspect of the invention described herein provides a method for quantifying effects of experiential exposure on stem cell maturation in a subject, including:

obtaining an exposure sample and a control sample from the subject and analyzing extent of methylation of at least one CpG dinucleotide in DNA of each sample within a plurality of CpG dinucleotide locations selected from at least one of the group of cg10338787, cg22497969, cg11968804, cg10237252, cg17310258, cg13485366, cg03455765, cg04193160, cg27367526, cg03384000, cg15575683, cg17471939, cg11199014, cg13948430, cg01567783, cg01278041, cg19005955, cg16154155, cg14652587, cg19659741, cg06705930, cg23009780, cg22130008, cg05840541, cg06953130, cg11194994, and cg14375747, thereby determining a methylation status of at least one CpG dinucleotide in the DNA of the exposure sample and a methylation status of at least one CpG dinucleotide in the DNA of the control sample; and,

deconvoluting the methylation array data from the control sample and the exposure sample to obtain methylation status of individual leukocyte subtypes in the samples, and comparing methylation status of the at least one CpG dinucleotide within a leukocyte subtype of the control sample to the methylation status of the at least one CpG dinucleotide within the same leukocyte subtype of the exposure sample, to determine sites of differential methylation, and correlating a difference in methylation status between the control sample and the exposure sample to obtain the effect of the exposure on stem cell methylation signature.

An aspect of the invention described herein provides a kit for quantifying embryonic cell from extent of hypermethylation, the kit including a plurality of primers for custom bisulfate sequencing library preparation, each primer directing amplification of a hyper methylatable CpG dinucleotide located in a DNA sequence selected from cg10338787, cg22497969, cg11968804, cg10237252, cg17310258, cg13485366, cg03455765, cg04193160, cg27367526, cg03384000, cg15575683, cg17471939, cg11199014, cg13948430, cg01567783, cg01278041, cg19005955, cg16154155, cg14652587, cg19659741, cg06705930, cg23009780, cg22130008, cg05840541, cg06953130, cg11194994, and cg14375747.

An aspect of the invention described herein provides an array for quantifying embryonic stem cell (ESC) content in a biological sample, including a surface containing a plurality of hypermethylatable CpG locations, the locations selected from at least one of the group of: cg10338787, cg22497969, cg11968804, cg10237252, cg17310258, cg13485366, cg03455765, cg04193160, cg27367526, cg03384000, cg15575683, cg17471939, cg11199014, cg13948430, cg01567783, cg01278041, cg19005955, cg16154155, cg14652587, cg19659741, cg06705930, cg23009780, cg22130008, cg05840541, cg06953130, cg11194994, and cg14375747, for analyzing ESC content having an ESC methylation signature.

### DETAILED DESCRIPTION OF EMBODIMENTS

Stem cell maturation is a fundamental, yet poorly understood aspect of human development. Fetal hematopoiesis is driven by embryonic stem cells (ESC) that give rise to adult hematopoietic stem cells (A-HSC) after birth and during the first years of life. Thus, postnatal development is marked by a dynamic temporal transition affecting all blood cellular elements. This developmental maturation of immune cells is accompanied by epigenomic remodeling of immune cells, including alterations in DNA methylation. Methylation of DNA on cytosines of CpG dinucleotides in the genome has long been known to be involved in regulation of gene expression (see Messerschmidt, D. M. et al., 2014, Genes Dev. 28: 812-828 for a review). There are about 28 million CpG sites in the human genome. However the particular patterns of methylation with respect to locations of CpG dincleotides in the genome, and the myriad patterns of gene expression that change during the variety of patterns of tissue differentiation and development, remains largely unknown.

Embodiments of the methods and compositions herein are based on DNA methylation patterns that might be used to trace the developmental history of immune cells during their maturation and reveal temporal and individual variations in the shift from ESC to A-HSC dependent hematopoiesis. A DNA methylation signature was devised that is deeply reminiscent of embryonal stem cells to interrogate the evolving character of multiple human tissues.

Examples described herein provide methods that harmonized adult (n=36) and newborn (n=151) isolated peripheral blood leukocyte subtypes (CD4, CD8, B-cell, NK, monocyte, granulocyte) and compared using linear mixed effect models adjusting for age, sex and subject, as a random effect. From the list of significant candidates (Q-value<0.05), a subset of highly invariant sites was identified. Using a constrained projection/quadratic programming approach the proportion of ESC or FCO signature in the samples was projected. The results of this example were replicated using 46 newborns and 200 adult isolated leukocyte samples. The results were further extended to observe if this signature was present in other cells using isolated embryonic and fetal hematopoietic cells (n=74) which were compared to adult bone marrow cells (n=49); fetal somatic (n=247) cells compared to adult somatic tissues (n=156), and cord blood (n=60) and peripheral blood samples (n=993) at different ages (0 to 103 years).

The results identified a common set of differentially methylated CpG sites that constitute a lineage invariant and developmentally sensitive methylation signature across the different leukocyte subtypes. The cell fraction displaying the signature was highly dependent upon developmental stage (fetal vs adult) and in leukocytes, it described a dynamic transition during the first 5 years of life. A dramatic loss of the ESC or FCO signature occurs in blood following birth with a 50% reduction occurring at approximately 1 year. After age 5 a low but detectable level of ESC occurs in some individuals even into advanced ages. Significant interindividual variation in ESC fraction at birth is partly explained by gestational age. Significant individual variation in the embryonic signature of leukocytes was evident at birth, in childhood, and throughout adult life. The embryonic origin of the newborn cells is supported by the highly concordant methylation signatures they share with embryonic stem cell lines, induced pluripotent cells and fetal liver CD34+ stem/progenitors. Furthermore, multiple non-hematopoietic fetal tissues but not their adult counterparts display the signature, thus confirming it as a marker of embryonic lineage. The ESC or FCO methylation signature provides insight into a fundamental developmental process of immune cell maturation. The genes denoting the signature included transcription factors and proteins intimately involved in embryonic development.

The examples herein determined the DNA methylation signature by the methods to trace the developmental origin of cells and informs the study of stem cell heterogeneity in humans under homeostatic and pathologic conditions. The FCO methylation assay provides a method to identify and quantitate an embryonic stem cell DNA methylation signature in human blood cells and non-hematopoietic tissues. The assay is a tool for characterizing the developmental maturation of human cells and tissues with a broad range of applications in clinical diagnostics, epidemiology, and stem cell related therapeutic products. Potential application areas include:

In human epidemiological research, the FCO methylation diagnostic assay is a research tool for epidemiologists and developmental biologists to gauge the extent of stem cell maturation in children to assess the effects of therapies (vaccines), nutrition, genetic variations, and other environmental exposures on normal developmental processes.

In prenatal diagnostics, the FCO methylation diagnostic assay is a research tool that determines variations in growth in utero linked to hematopoietic stress (e.g. pre-eclampsia) and congenital abnormalities (e.g. Downs syndrome).

In non-human veterinary toxicology and pre-clinical animal model studies the FCO methylation diagnostic assay is a research tool for discovery, validation, and library deconvolution, for example, to be transferred to the mouse to create the first efficient stem cell maturation tool to study maturation in all murine tissues. Toxicological testing in mice could be targeted to stem cell maturation using the FCO signature to reveal potentially harmful chemical agents that would be identified before they are marketed.

In hematology oncology medical practice the FCO methylation diagnostic assay provides the FCO methylation signature which has value to assess progress of patients treated with G-CSF and similar agents that promote granulopoiesis during cancer therapy induced neutropenia. Induced granulopoiesis shows different stem cell characteristics that predict function of the resulting cells.

In transplantation medicine the FCO methylation diagnostic assay provides the ESC methylation signature to be used during induction of CD34 stem progenitors in transplantation medicine to indicate the quality and extent of the induction process.

In stem cell therapeutics and regenerative medicine the FCO methylation diagnostic assay provides the ESC methylation signature which is to be used as a quality control measure during the reprogramming of adult cells into induced pluripotent stem cells (iPCS), which are to be used following their differentiation in regenerative medicine applications. The FCO signature of adult cells would revert to an embryonal form as a result of an efficient reprogramming process. At present there are dozens of different reprogramming protocols being evaluated and little to guide their success. There is a need for methods to evaluate reprogramming of adult cells into pluripotent stem cells.

The provisional application filed Sep. 26, 2017 Ser. No. 62/563,354 from which this application claims the benefit of priority included as an appendix a manuscript entitled, “Tracing human stem cell lineage during development using DNA methylation”, co-authors Lucas A. Salas, John K. Wiencke, Devin C. Koestler, Brock C. Christensen, and Karl T. Kelsey. This manuscript has been published as Salas et al., in Genome Research, Cold Spring Harbor Laboratory Press, Aug. 20, 2018. The provisional application 62/563,354 and the published paper by Salas et al. 2018 are hereby incorporated herein by reference in their entireties.

A spread sheet of a table of nucleotide sequences of the 27 CpG sites determined in the examples herein to have methylation differences between umbilical cord blood (UCV) and adult whole blood (AWB) which were observed to be consistent across all cell types was included as an Appendix in provisional application 62/563,354, and is found in in Salas et al., 2018. Genome Biol 19: 64, which is hereby incorporated herein by reference in its entirety. The identifying information in this table has 27 lines and 24 columns as follows from left to right. Column 1 on the left is a code name of a CpG site; column 2 gives the chromosome location; column 3 gives the start site according to hg19; column 4 gives the end site according to hg19; columns 5 and 6 respectively give the start and end sites, respectively according to hg38; columns 7 and 8 indicate the strand and orientation in which the sequence extends upstream (reverse, negative or 5′ orientation, indicated “up”), or downstream (forward, positive or 3′ orientation, indicated “down”); columns 9-12 give details of channel design according to either Infinium II design (both channels) or Infinium I design (Red or Green), the next base and the next base reference; column 13-16 gives the probe starts and ends in hg19 and hg 38, respectively; column 18 contains the nucleotide sequences of probes of ProbeSeqA (SEQ ID Nos: 1-27); column 20 gives the nucleotide sequences of probes of ProbeSeqB (SEQ ID Nos: 28-31); column 22 gives the nucleotide sequences SEQ ID Nos: 32-58 of the SourceSeq which are the original sequences prior to bisulfite conversion used for probe design; and column 24 shows the nucleotide sequences SEQ ID Nos: 59-85, of the Forward Sequence Plus (+) Strand 5′-3′ (HapMap) 5′-3′ flanking the CG which is identified by square brackets. The relevant sequences referred to in the claims and specification accordingly are identified by the SEQ ID Nos for each probe code in column 20. The format of the spread sheet as attached hereto is separated into 5 pages formatting the data from left to right to include information found in the original spreadsheet.

Following fertilization, DNA methylation is erased and reestablished in concert with lineage commitment and cellular differentiation (Lee H J et al. 2014. Cell Stem Cell 14: 710-9. As lineage specific marks of DNA methylation have been successfully employed to detect the relative abundance of individual cell types in blood mixtures (Houseman E A et al. 2012. BMC Bioinformatics 13: 86; Accomando W P et al. 2014. Genome Biol 15: R50; Koestler D C et al. 2016. BMC Bioinformatics 17: 120; Salas L A et al. 2018. Genome Biol 19: 64) and because a significant proportion of progenitor and stem cell methylation events are mitotically stable throughout differentiation, it is possible that a common set of unchanging DNA methylation markers can trace a common cell ontogeny (Kim K et al. 2010. Nature 467: 285-90).

Analytical methods and devices are provided herein that involve generating a library of stable CpG loci that are markers of the cell of origin for studying peripheral blood leukocytes. The methods are based upon the observation that a subset of CpG-specific methylation marks is inherited in progeny cells irrespective of lineage differentiation. These candidate marker loci, reflecting the progenitors from which they are derived, are identified and selected as an initial step in assembling the devices and method. In a second filtering process, a subset of these candidate loci is selected that optimizes the discrimination of fetal and adult differentiated leukocytes. This second step provides CpG marker loci that are different among fetal and adult progenitors; these loci are used herein to form a fetal cell origin (FCO) signature. The FCO signature is employed in conjunction with methods and processes for cell mixture deconvolution (Houseman et al. 2012, herein) for estimating the proportion of cells in a mixture of cell types that are of fetal cell origin.

### EXAMPLES

The following methods were used thoughout the examples.

### Example 1. Discovery Datasets

For the discovery of CpG markers, three public available datasets were used containing purified cell types (granulocytes: Gran, CD14⁺ monocytes: Mono, CD19⁺ B lymphocytes: Bcell, CD4⁺ T lymphocytes: CD4T, CD8⁺ T lymphocytes: CD8T, and CD56⁺ natural killer lymphocytes: NK cells) from peripheral blood in adults and cord blood in newborns were used (see Table 1). Discovery datasets contained whole blood and purified cell subtypes from several subjects: 1) GSE35069 (Reinius L E et al. 2012. PLoS One 7) contained purified cells from six adult subjects. 2) FlowSorted.CordBlood.450K (Bakulski K M et al. 2016. Epigenetics 11: 354-362) contained samples, from 17 newborns. 3) FlowSorted.CordBloodNorway.450K (Gervin K et al. 2016. Epigenetics 2294: 00-00) contained samples from 11 newborns.

Table 1 first part is a one page summary of data sources and citations. A second part of Table 1 contains data for a list of 834 candidate loci detected, and includes the final 27 selected CpG sites. The following information for each of the 834 sites, respectively, is given on pages 1-55: cgid; gene name; CHR (chromosome) Coordinates (according to build hg19), enhancer, and genomic context. The following information for each of these 834 respective sites is given on pages 56-110: mean methylation adult; mean methylation cord blood; Δ β; selected 27; β coefficient linear mixed model; P-value and FDR. The following information for each of these 834 respective sites is given on pages 111-165: functions; and transcripts.

TABLE 1
Data sources and citations
Discovery and validation datasets

| | | | | | Myeloid |
|---|---|---|---|---|---|
| | Lymphocytes | | | | cells |
| | Bcell | CD4T | CD8T | NK | Gran Ficoll |
| Repository | CD19⁺ | CD4⁺ | CD8⁺ | CD56⁺ | recovery |

Discovery datasets

| | | | | | | |
|---|---|---|---|---|---|---|
| Umbilical | FlowSorted.CordBlood.450K (Bakulski et al. 2016) | 15 | 15 | 14 | 14 | 12 |
| cord blood | FlowSorted.CordBloodNorway.450K (Gervin et al. 2016) | 11 | 11 | 11 | 11 | 11 |
| Peripheral | GSE35069 (Reinius et al. 2012) | 6 | 6 | 6 | 6 | 6 |
| blood | | | | | | |

Replication datasets

| | | | | | | |
|---|---|---|---|---|---|---|
| Umbilical | GSE68456 (de Goede et al. 2015) | 7 | 7 | 6 | 6 | 7 |
| cord blood | GSE30870 (Heyn et al. 2012) | 0 | 1 | 0 | 0 | 0 |
| Peripheral | GSE59065 (Tserel et al. 2015) | 0 | 99 | 100 | 0 | 0 |
| blood | GSE30870 (Heyn et al. 2012) | 0 | 1 | 0 | 0 | 0 |

Discovery and validation datasets

| Myeloid | | | | |
|---|---|---|---|---|
| cells | Subjects | | | |
| Mono | Fe- | | | |
| CD14⁺ | males | Males | Total | Age mean(SD) |

Discovery datasets

| | | | | | | |
|---|---|---|---|---|---|---|
| Umbilical | FlowSorted.CordBlood.450K (Bakulski et al. 2016) | 15 | 7 | 8 | 15 | 39.9(1.0) weeks |
| cord blood | FlowSorted.CordBloodNorway.450K (Gervin et al. 2016) | 11 | 6 | 5 | 11 | 39.3(1.2) weeks |
| Peripheral | GSE35069 (Reinius et al. 2012) | 6 | 0 | 6 | 6 | 38 (13.6) years |
| blood | | | | | | |

Replication datasets

| | | | | | | |
|---|---|---|---|---|---|---|
| Umbilical | GSE68456 (de Goede et al. 2015) | 12 | 7 | 5 | 12 | Term newborns |
| cord blood | GSE30870 (Heyn et al. 2012) | 0 | NA | NA | 1 | Term newborn |
| Peripheral | GSE59065 (Tserel et al. 2015) | 0 | 52 | 48 | 100 | 52.6(23.7) |
| | | | | | | years |
| blood | GSE30870 (Heyn et al. 2012) | 0 | NA | NA | 1 | 103 years |

| | Repository | Whole blood | Females | Males | Total | Age mean(SD) |
|---|---|---|---|---|---|---|
| AUROC datasets | | | | | | |
| Umbilical 0cord blood | GSE80310 | 24 | 13 | 11 | 24 | Term (38.142.9 weeks) |
| | (Knight et al. 2016) | | | | | newborns |
| | GSE74738 | 1 | 0 | 0 | 1 | Pooled sample (Unknown |
| | (Hanna et al. 2016) | | | | | gestational age) |
| | GSE54399 | 24 | 10 | 14 | 24 | Term newborns, with |
| | (Montoya-Williams et al. 2017) | | | | | unknown health conditions |
| | | | | | | rural war area |
| | GSE79056 | 36 | 19 | 17 | 36 | 14 preterm (24.1-34 |
| | (Knight et al. 2016) | | | | | weeks), 22 term (39- |
| | | | | | | 40.9 weeks) newborns |
| | GSE62924 | 38 | 22 | 16 | 38 | 39 (1.4) weeks |
| | (Rojas et al. 2015) | | | | | |
| Peripheral blood | GSE74738 | 10 | 10 | 0 | 10 | 29.0 (9.7) years (healthy |
| | (Hanna et al. 2016) | | | | | women) |
| | GSE54399 | 24 | 24 | 0 | 24 | 32.8 (7.4) years (unknown |
| | (Montova-Williams et al. 2017) | | | | | health conditions rural |
| | | | | | | war area) |
| Synthetic mixtures datasets | | | | | | |
| Umbilical cord blood | GSE66459 | 22 | 11 | 11 | 22 | 11 Term (38-41 weeks) and |
| | (Fernando et al. 2015) | | | | | 11 preterm newborns (26- |
| | | | | | | 36 weeks) |
| Peripheral blood | GSE43976 | 52 | 52 | 0 | 52 | 42.2(8.4) years (healthy |
| | (Marabita et al. 2013) | | | | | women) |

Embryonic stem cells, induced Plurinotent stem cells and hematopoietic cell progenitors**

| | | | CD34⁺ | Erythroid | CD34⁺ | | | | | |
|---|---|---|---|---|---|---|---|---|---|---|
| Repository | ESC | iPSC | fetal | fetal | Adult | MPP | L-MPP | CMP | GMP | MEP |
| GSE31848 (Nazor et al. 2012) | 19 | 29 | 0 | 0 | 0 | 0 | 0 | 0 | 0 | 0 |
| GSE40799 (Weidner et al. 2013) | 0 | 0 | 3 | 0 | 0 | 0 | 0 | 0 | 0 | 0 |
| GSE56491 (Lessard et al. 2015) | 0 | 0 | 0 | 12 | 0 | 0 | 0 | 0 | 0 | 0 |
| GSE56491 (Lessard et al. 2015) | 0 | 0 | 0 | 0 | 0 | 0 | 0 | 0 | 0 | 0 |
| GSE50797 (Rönnerblad et al. | 0 | 0 | 0 | 0 | 0 | 0 | 0 | 3 | 3 | 0 |
| 2014) | | | | | | | | | | |
| GSE63409 (Jung et al. 2015) | 0 | 0 | 0 | 0 | 5 | 5 | 5 | 5 | 5 | 5 |

Embryonic stem cells, induced Plurinotent stem cells and hematopoietic cell progenitors**

| | Erythroid | | | | | | |
|---|---|---|---|---|---|---|---|
| Repository | adult | PMC | PMN | Females | Males | Total | Age |
| GSE31848 (Nazor et al. 2012) | 0 | 0 | 0 | 42 | 12 | 54 | NA |
| GSE40799 (Weidner et al. 2013) | 0 | 0 | 0 | NA | NA | 3 | Term newborns |
| GSE56491 (Lessard et al. 2015) | 0 | 0 | 0 | NA | NA | 12 | Abortuses |
| GSE56491 (Lessard et al. 2015) | 12 | 0 | 0 | NA | NA | 12 | Adult bone |
| | | | | | | | marrow |
| GSE50797 (Rönnerblad et al. | | 3 | 3 | 1* | 2* | 3* | Adult bone |
| 2014) | | | | | | | marrow |
| GSE63409 (Jung et al. 2015) | 0 | 0 | 0 | 2* | 3* | 5* | 22-43 years |

Somatic tissues

| | Repository | Adrenal | Brain | Heart | Liver | Lung | Muscle | Pancreas | Spleen |
|---|---|---|---|---|---|---|---|---|---|
| Fetal | GSE61279 (Bonder et al. 2014) | 0 | 0 | 0 | 14 | 0 | 0 | 0 | 0 |
| | GSE31848 (Nazor et al. 2012) | 3 | 4 | 4 | 4 | 5 | 0 | 0 | 3 |
| | GSE56515 (Slieker et al. 2015) | 9 | 0 | 0 | 0 | 0 | 9 | 8 | 0 |
| | GSE58885 (Spiers et al. 2015) | 0 | 179 | 0 | 0 | 0 | 0 | 0 | 0 |
| Adult | GSE61279 (Bonder et al. 2014) | 0 | 0 | 0 | 96 | 0 | 0 | 0 | 0 |
| | GSE31848 (Nazor et al. 2012) | 2 | 1 | 1 | 0 | 2 | 2 | 2 | 2 |
| | GSE48472 (Slieker et al. 2013) | 0 | 0 | 0 | 5 | 0 | 6 | 4 | 3 |
| | GSE41826 (Guintivano et al. 2013) | 0 | 29 | 0 | 0 | 0 | 0 | 0 | 0 |

Somatic tissues

| | | | Subjects | | | |
|---|---|---|---|---|---|---|
| | Repository | Stomach | Females | Males | Total | Age |
| Fetal | GSE61279 (Bonder et al. 2014) | 0 | NA | NA | 14 | 8-21 weeks |
| | GSE31848 (Nazor et al. 2012) | 5 | 4* | 2* | 6* | 14, 15, 18, and 20 weeks |
| | GSE56515 (Slieker et al. 2015) | 0 | NA | NA | 10* | 9, 18 and 22 weeks |
| | GSE58885 (Spiers et al. 2015) | 0 | 79 | 100 | 179 | 3-26 weeks |
| Adult | GSE61279 (Bonder et al. 2014) | 0 | 48 | 48 | 96 | 26.8 (10.5) years |
| | GSE31848 (Nazor et al. 2012) | 1 | 2* | 1* | 3* | 48.0 (8.5) years |
| | GSE48472 (Slieker et al. 2013) | 0 | NA | NA | 6* | 52.5 (7.5) years |
| | GSE41826 (Guintivano et al. 2013) | 0 | 15 | 14 | 29 | 33.3 (17.2) years |

| Aging | | Whole | Mononuclear | | | | |
|---|---|---|---|---|---|---|---|
| datasets | Permanent repository | blood | cells | Females | Males | Total | Age |
| Umbilical | FlowSorted.CordBlood.450K (Bakulski et al. 2016) | 15 | 0 | 8 | 7 | 15 | 38.9 (1.3) weeks |
| cord blood | FlowSorted.CordBloodNorway.450K (Gervin et al. 2016) | 11 | 0 | 6 | 5 | 11 | 39.3 (1.2) weeks |
| Peripheral | GSE30870 (Heyn et al. 2012) | 0 | 19 | NA | NA | 19 | 38.7 (1.9) weeks |
| blood | GSE83334 (Urdinguio et al. 2016) | 15 | 0 | 9 | 6 | 15 | 38.9 (1.4) weeks |
| | GSE62219 (Acevedo et al. 2015) | 60 | 0 | 60 | 0 | 60 | 2.3 (1.7) years |
| | GSE36054 (Alisch et al. 2012) | 134 | 0 | 55 | 79 | 134 | 4.6 (4.1) years |
| | GSE40279 (Hannum et al. 2013) | 656 | 0 | 338 | 318 | 656 | 64.0 (14.7) years |
| | GSE35069 (Reinius et al. 2012) | 6 | 6 | 0 | 6* | 6* | 38 (13.6) years |
| | GSE30870 (Heyn et al. 2012) | 0 | 19 | NA | NA | 19 | 92.6 (3.7) years |
| | GSE59065 (Tserel et al. 2015) | 97 | 0 | 49 | 48 | 97 | 52.7 (23.7) years |
| | GSE83334 (Urdinguio et al. 2016) | 15 | 0 | 9 | 6 | 15 | 5 years |

*Several samples were drawn from the same subject
**ESC: undifferentiated embryonic stem cells, iPSC: undifferentiated induced pluripotent stem cells, CD34⁺ fetal: stem/progenitor cells from fresh umbilical cord blood, erythroid fetal and adult: CD34⁺ cells from fetal liver and bone marrow respectively differentiated ex-vivo to erythroid cells (transferrin receptor-CD71⁺, and glycophorin-CD235α⁺), CD34⁺ adult: CD34⁺CD38⁻ CD90⁺CD45RA⁻, adult bone marrow progenitors samples: MPP-multipotent progenitors CD34⁺CD38⁻CD90⁻CD45RA⁻, L-MPP—lymphoid primed multipotent progenitors CD34⁺CD38⁻CD90⁻CD45RA⁺, CMP—common myeloid progenitors CD34⁺CD38⁺CD123⁺CD45RA⁻, GMP—granulocyte/macrophage progenitors CD34⁺CD38⁺CD123⁻CD45RA⁺, MEP—megakaryocyte-erythroid progenitors CD34⁺CD38⁺CD123⁻CD45RA⁻, CD34⁺ myeloid progenitors: CMP—common myeloid progenitors CD34⁺CD38⁺CD123⁺CD110⁻CD45RA⁻, and GMP—granulocyte/macrophage progenitors CD34⁺CD38⁺CD123⁺CD110⁻CD45RA⁺, CD34⁻ immature myeloid progenitors: PMC—promyelocyte/myelocyte CD34⁻ CD117⁺CD33⁺CD13⁺CD11b⁺, PMN—metamyelocyte/band-myelocyte CD34⁻ CD117⁻ CD33⁺CD13⁺CD11b⁺.

Table 2 shows developmentally sensitive methylation signature deconvolution in each of pluripotent cells, fetal progenitor cells, and adults CD34+ stem/progenitor cells.

TABLE 2
Fetal Cell Origin (FCO) signature deconvolution in pluripotent,
fetal progenitors and adult CD34⁺ stem/progenitor cells.

| Fetal/embryonic | Cell Type | N | mean (SD) | |
|---|---|---|---|---|
| Fetal/embryonic | ESC | 25 | 75.1 | (9) |
| | iPSC | 29 | 81 | (1.9) |
| | CD34+ fetal | 3 | 81.8 | (2.3) |
| | Erythroid fetal | 12 | 63.6 | (3.3) |
| | CD34+ adult | 5 | 12.1 | (6.7) |
| | MPP | 5 | 2.6 | (3.8) |
| | L-MPP | 5 | 4.3 | (4.5) |
| | CMP | 8 | 4.4 | (3.7) |
| Adult | GMP | 8 | 4.8 | (6.4) |
| progenitors | MEP | 5 | 4.2 | (4.5) |
| (bone marrow) | Erythroid adult | 12 | 2.8 | (3.8) |
| | PMC | 3 | 2.7 | (4.7) |
| | PMN | 3 | 2.1 | (3.7) |

Estimated mean (SD) FCO methylation fractions for embryonic/fetal cells are 75.9% (8.5) and 4.4% (5.1) for adult progenitors (bone marrow), P = 1.81 × 10⁻⁸⁶.
Abbreviations: Embryonic stem cells (ESC), Induced Pluripotent Stem cells (iPSC), CD34⁺ fetal (fresh cord blood cells expressing CD34⁺), Erythroid fetal (fetal liver CD34⁺ cells, differentiated ex vivo to express transferrin receptor and glycophorin), CD34⁺ adult (bone marrow expressing CD34⁺ CD38⁻ CD90⁺ CD45RA⁻), Multipotent progenitors (MPP), Lymphoid primed multipotent progenitors (L-MPP), Common myeloid progenitors (CMP), Granulocyte/macrophage progenitors (GMP), Megakaryocyte-erythroid progenitors (MEP), Erythroid adult (adult bone marrow CD34⁺ cells, differentiated ex vivo to express transferrin receptor and glycophorin), Promyelocyte/myelocyte (PMC), metamyelocyte/band-myelocyte (PMN).

### Example 2. Biomarker Discovery: Creation of a Lineage Invariant and Developmentally Sensitive DNA Methylation Signature (the Fetal Cell Origin-FCO Signature)

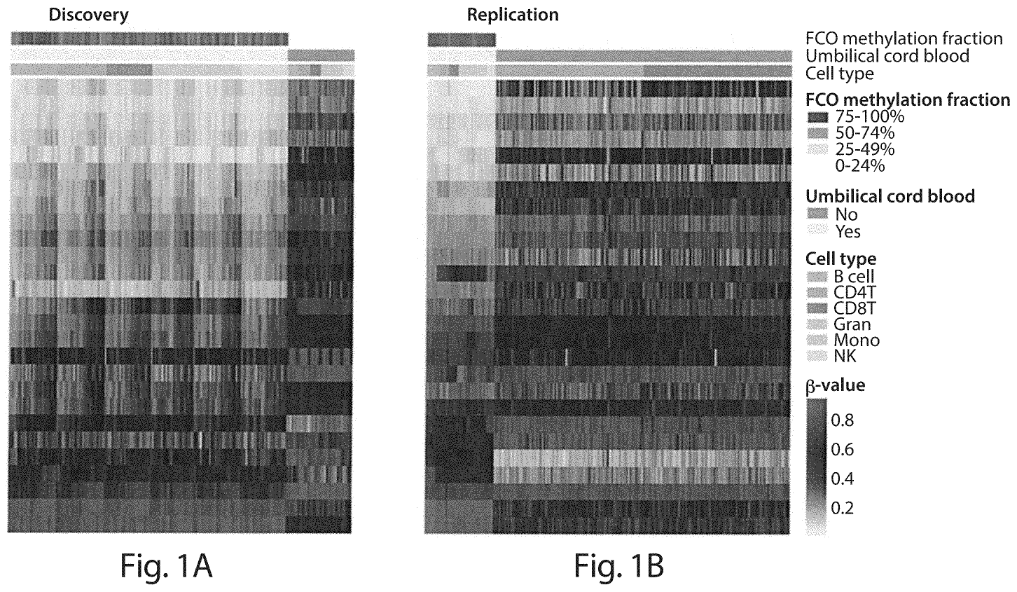

It was envisioned in examples herein that embryonic and adult hematopoietic stem cells contain CpG loci that are unique to each of these types of stem cells and that are invariant with respect to the lineage specification of their progeny. Thus, a selection strategy was undertaken in two steps: using discovery datasets, first lineage invariant CpG sites were indentified within isolated leukocyte populations from umbilical cord blood (UCB, fetal cells) and in adult whole blood (AWB), and second, among these CpG loci, a subset was identified that provided optimal discrimination between all subtypes of UCB and adult leukocytes (FIG. 1A and FIG. 1B).

The aforementioned three datasets were pooled and included purified Gran, Mono, Bcell, CD4T, CD8T, and NK cells only. Datasets were harmonized to include sex, DNA methylation age (Horvath S. 2013. Genome Biol 14: R115; Lowe D et al. 2016. Oncotarget 7: 8524-31), and a subject indicator. Horvath's DNA methylation age was calculated using the agep function in the wateRmelon R-package (Pidsley R et al. 2013. BMC Genomics 14: 293). For newborns, the Knight's DNA methylation gestational age was estimated (Knight A K et al. 2016. Genome Biol 17: 206). The pooled dataset was normalized using Funnorm (Fortin J et al. 2014. Genome Biol 15: 503). Once normalized, CpG loci exhibiting differential patterns of methylation between newborns and adults were identified using two similar but distinct approaches. In the first approach, series linear models adjusted for sex and sample specific estimated DNA methylation age, were fit independently to each of the J CpGs and to each cell type separately (Equation 1).

Yᵢⱼ^{(k)}=α₀ⱼ^{(k)}+α₁ⱼ^{(k)}I(tissueᵢ=fetal)+α₂ⱼ^{(k)}sexᵢ+α₃ⱼ^{(k)}DNAm Ageᵢ+ϵᵢⱼ^{(k)} Equation 1

In Equation 1, Yᵢⱼ^{(k)} represents the methylation β-value among subject i (i=1,2, . . . , N), CpG j (j=1,2, . . . , J), and cell type k (k=1,2, . . . , K). For each of the J×K models that were fit, the model that the mean methylation β-value is equivalent between fetal and adult tissues was tested (e.g., H₀: α₁ⱼ^{(k)}=0), and CpG loci exhibiting a statistically significant difference (FDR<0.05) were retained. In the second approach, a series linear mixed effect models adjusted for sex, sample specific estimated DNA methylation age, cell type (to obtain invariant loci across cell types), and including a subject-specific random intercept, were used to identify differentially methylated CpG loci between adult vs fetal tissues (Equation 2).

$${Y}_{ij} = {β}_{0 j} + {β}_{1 j} I ( {tissue}_{i} = fetal ) + {β}_{2 j} {sex}_{i} + {β}_{3 j} DNAm {Age}_{ij} + {\sum }_{k = 1}^{K} {γ}_{kj} I ( {celltype}_{ij} = k ) + {b}_{i} + {ϵ}_{ij} \qquad Equation 2$$

For each of the J fitted models, the model that the mean methylation β-value is equivalent between fetal and adult tissues (e.g., H₀: β₁ⱼ=0) was tested, and CpG loci exhibiting a statistically significant differences (FDR<0.05) were retained for further analysis. While the strategy for identifying developmentally variant loci involved fitting a series of linear regression and linear mixed effects models, treating the methylation p-values as the response, the existence of alternative models (Saadati M et al. 2014. StatMed 33: 5347-5357; Du P et al. 2010. BMC Bioinformatics 11: 587) that could be used as a substitute or in addition to the models considered here were considered equivalent. These equations are statistical tools that were developed to analyze a large number of data points for the purpose of developing methods of obtaining embryonic stem cell DNA methylation signatures.

The results of the seven models (e.g., six linear models, one fit to each cell type, along with the linear mixed effects model) were compared to identify CpG loci exhibiting statistically significant (FDR<0.05) differences between fetal and adult tissues across all seven models (1,255 CpG loci). Of those, CpG loci exhibiting inconsistent patterns of differential methylation fetal and adult tissues across any of two the seven models were filtered out. This process of identification and filtering out resulted in obtaining a set of loci that exhibited consistent patterns of differential methylation across all cell types. Among those, loci were prioritized that showed absolute differences in methylation between fetal vs adult tissues greater than 0.1 across all cell types (1,218 CpGs).

The filtered candidate CpG list was then subject to a test for enrichment to identify biological pathways enriched with the associated genes using the MSigDB v6.0 curated database 2 using three different approaches: 1) ToppGene which uses a classical hypergeometric distribution test (Chen J et al. 2009. Nucleic Acids Res 37: 305-311), 2) GREAT v3.0.0 (Genomic Regions Enrichment of Annotations Tool) (McLean C Y et al. 2010. Nat Biotechnol 28: 495-501) which interrogates potential cis-regulatory regions (5000 bp upstream and 1000 bp downstream, and an extended region 1 Mbp of the CpG site) that are not captured using the genes associated to the CpG site, and 3) the R-package missMethyl to account for the potential microarray bias (Phipson B et al. 2016. Bioinformatics 32: 286-8). To mitigate the potential for bias, the background was restricted to consider only those genes interrogated in the Illumina HumanMethylation 450K array. The pathways that overlap among the three approaches were selected. In addition, ToppGene was used to test for enrichment of loci on the Progenitor Cell Biology Consortium database (Chen J et al. 2009. Nucleic Acids Res 37: 305-311; Salomonis N et al. 2016. Stem Cell Reports 7: 110-125).

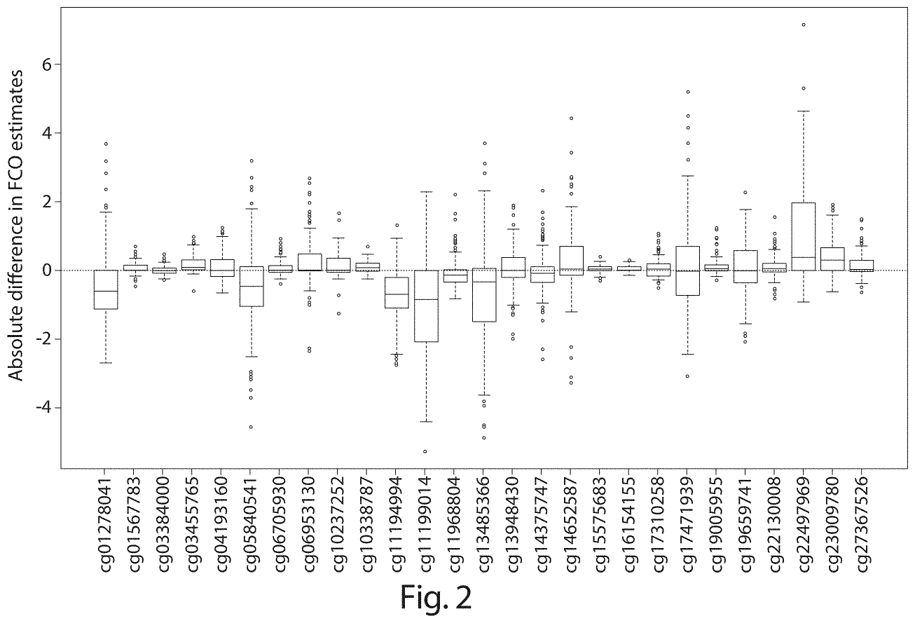

A next step involved reducing the candidate CpGs to a short instrumental list that provided optimal discrimination between adult and fetal tissues but minimal residual cell-specific effects. For this step, a confirmatory principal component (PC) analysis was used to quantitatively compare differences in the components of the candidate list. The first PC should account for differences between adult and fetal cells whereas subsequent PCs should account for inter-subject variability, residual cell type confounding, and other sources of technical noise. Indeed, using the methods herein it was observed that the first PC associated strongly with origin of the cell type (i.e., fetal versus adult), whereas the second PC indicated a small, but noticeable cell-specific effect (FIG. 2). To identify loci with residual cell-specific effects, the geometric angle was computed between the x-axis (direction of the first PC) and the vector formed by loadings for PC1 (x) and PC2 (y) for each CpG. The geometric angle calculation uses x and y as the legs of the triangle, and then, using the inverse trigonometric function arctangent (a tan), the geometric angle is obtained as degrees=a tan(x/y)×(180/r) with a known distribution between −90 and +90. CpGs with angles close to zero degrees represent those predominantly influencing PC1 (i.e. fetal versus adult differences), whereas angles away from zero degrees are indicative of contribution to PC2 (i.e., cell-specific effects). To minimize cell-specific signal among CpGs, only those CpGs whose angle was close to 0 degrees were selected to form the FCO signature.

Using the derived FCO signature, the fetal vs adult cell fraction was deconvoluted using constrained projection quadratic programming (CP/QP) proposed by Houseman (Houseman et al. 2012, herein), substituting the default reference library with the library identified based on the above analysis (Provisional application 62/563,354, and Salas et al., 2018, herein, both of which are hereby incorporated herein by reference in their entireties). For analyses using GEO datasets, no additional normalization steps were employed to the already preprocessed β-values. β-value distributions were however inspected for irregularities, and where relevant, k nearest neighbors was performed for missing value imputation.

### Example 3. Replication

Purified Gran, Mono, Bcell, CD4T, CD8T, and NK were used from three replication datasets: GSE68456 (de Goede O M et al. 2015. Clin Epigenetics 7: 95) included samples from cord blood of 12 newborns; GSE30870 (Heyn H, et al. 2012. Proc NatlAcad Sci USA 109: 10522-10527) contains purified CD4T of one adult and one newborn; and 3GSE59065 (Tserel L et al. 2015. Sci Rep 5: 13107) included 99 CD4T, and 100 CD8T samples.

### Example 4. AUROC, Stability of the FCO Estimations and Synthetic Mixture Statistical Validation

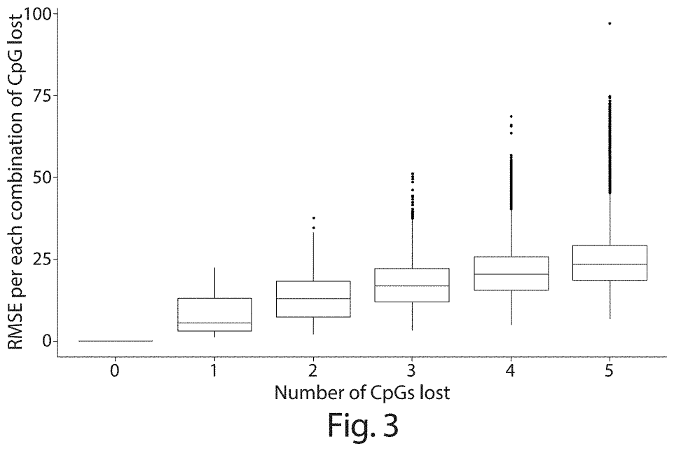

Five independent datasets were used to evaluate the classification area under the ROC (AUROC) curve of the FCO signature and the stability of the FCO estimations. To establish the stability of the FCO signature, the absolute difference in the FCO estimates were evaluated when all the potential combinations of one to five CpGs were lost during the FCO estimations compared to the full set of 27 CpGs using the samples used for the AUROC analysis (umbilical cord blood GSE80310 (Knight et al. 2016, herein), GSE74738 (Hanna C W et al. 2016. Genome Res 26: 756-67), GSE54399 (Montoya-Williams D et al. 2017. JDev Orig Health Dis 9: 1-8), GSE79056 (Knight et al. 2016, herein), GSE62924 (Rojas D et al. 2015. Toxicol Sci 143: 97-106). Adult peripheral blood GSE74738 (Hanna et al. 2016, herein), GSE54399 (Montoya-Williams et al. 2017, herein). The average root mean square error (RMSE) was also calculated between the prediction using the 27 CpGs compared to all the potential combinations when as few as one CpG and as many as five CpGs were excluded from the 27 FCO CpGs. The data indicated that the set of 27 CpG sites is a minimum discriminatory set for a reliable FCO estimation. See FIG. 2 and FIG. 3.

Within the 27 CpGs the loss of eight probes (cg01278041, cg05840541, cg11194994, cg11199014, cg13485366, cg14652587, cg17471939, cg22497969) had the biggest impact in the FCO calculations (RMSE>10). In contrast the loss of some other probes (e.g. cg01567783, absent in the EPIC array), altered only minimally the FCO estimates (RMSE:2.24). The full set of 27 probes were used herein for further assays and determinations. In the absence of specific probes the increase in the estimation errors should be considered.

In the x axis 0 corresponds to the reference including the 27 CpGs, 1, corresponds to 27 combinations losing one CpG, 2 to 351 combinations losing 2 CpGs, 3 to 2925 combinations losing 3 CpGs, 4 to 17550 combinations losing four CpGs, and 5 to 80730 combinations losing 5 CpGs: GSE80310 (Knight et al. 2016, herein), GSE74738 (Hanna et al. 2016, herein), GSE54399 (Montoya-Williams et al. 2017, herein), GSE79056 (Knight et al. 2016, herein), GSE62924 (Roj as et al. 2015, herein). To simulate synthetic mixtures two additional DNA methylation data sets were used: GSE66459 a fetal UCB (n=22) data set (Femando F et al. 2015. BMC Genomics 16: 736) and GSE43976 restricting to those samples of adult peripheral blood (n=52) data set (Marabita F et al. 2013. Epigenetics 8: 333-46).

To establish the reliability of the fetal deconvolution methodology provided in examplese herein, an additional example was performed that involved first creating, and then deconvoluting synthetic mixtures of fetal UCB and adult peripheral blood DNA methylation profiles mixed in in predetermined proportions. More precisely, let S^{CB} and S^{A} represent J×1 vectors of methylation β-values for fetal UCB and adult peripheral blood (Fernando et al. 2015, herein; Marabita et al. 2013, herein), respectively, with J denoting the number of CpG loci.

The synthetic mixture, M, was generated as weighted linear combination of SC^{B} and S^{A}, such that: M=πS^{CB}+(1−π) S^{A} and 0≤π≤1. Assuming that S^{CB} and S^{A} represent the DNA methylation profile over “pure” populations of fetal and adult cells, respectively, π represents the fraction of cells carrying the FCO signature within the synthetic mixture, M. Application of cell mixture deconvolution to M using the FCO signature library allowed estimation of the fraction of cells carrying the FCO signature, \hat{π}, which was compared to the “known” predetermined proportion, it.

To simulate synthetic mixtures two additional DNA methylation data sets were used: GSE66459 a fetal UCB (n=22) data set (Fernando et al. 2015, herein) and GSE43976 restricting to those samples of adult peripheral blood (n=52) data set (Marabita et al. 2013, herein). Importantly, neither of these data sets was used to identify or derive the FCO signature that forms the basis of deconvolution, and therefore represent truly independent data sets. Synthetic mixtures were generated by mixing randomly selected samples from both the fetal UCB and adult peripheral blood data sets, where the mixing parameter was selected to be π={0.1, 0.2, 0.3, 0.4, 0.5, 0.6, 0.7, 0.8, 0.9}. For each specification of π, n=10 synthetic mixture were generated.

### Example 5. Embryonic Stem Cells (ESC), Induced Pluripotent Stem Cells (iPSC) and Hematopoietic Cell Progenitors

To analyze the ontogeny of the stem cell methylation signature several databases of arrayed hematopoietic progenitors were determined: GSE31848 (Nazor K L et al. 2012. Cell Stem Cell 10: 620-634) undifferentiated embryonic stem cells (ESC) (n=19) and induced pluripotent stem cells (iPSC) (n=29); GSE40799 (Weidner C I et al. 2013. Sci Rep 3: 3372), three fresh CD34⁺ stem/progenitor cells from fresh umbilical cord blood; GSE56491 (Lessard S et al. 2015. Genome Med 7: 1), 12 CD34⁺ cells from fetal liver and 12 from adult bone marrow, which were differentiated ex-vivo to erythroid cells; GSE50797 (Ronnerblad M et al. 2014. Blood 123: e79-89) three adult bone marrow samples were used to isolate two different CD34⁺ myeloid progenitors (CMP—common myeloid progenitors, and GMP-granulocyte/macrophage progenitors) and two different CD34⁻ immature myeloid progenitors (PMC-promyelocyte/myelocyte, and PMN—metamyelocyte/band-myelocyte); and, GSE63409, (Jung N et al. 2015. Nat Commun 6: 8489) five adult bone marrow samples including six different isolated CD34⁺ progenitors (CD34⁺ adult stem cells, MPP-multipotent progenitors, L-MPP-lymphoid primed multipotent progenitors, CMP—common myeloid progenitors, GMP-granulocyte/macrophage progenitors, MEP-megakaryocyte-erythroid progenitors), see Table 1.

### Example 6. Fetal/Embryonic and Adult Somatic Tissue

The FCO methods and processes were applied to data from non-hematopoietic tissues to explore the specificity of the DNA methylation signature among tissues derived from diverse embryonic layers and progenitors. For this purpose six additional datasets restricting to those organs with at least one adult (necropsies) and one fetal (abortuses) sample were included (see Table 1): GSE61279 (Bonder M J, et al. 2014. BMC Genomics 15: 860), liver samples (fetuses n=14, adults n=96); GSE31848 (Nazor et al. 2012, herein), different organ biopsies (fetal n=28, adults n=13); GSE56515 (Slieker R C et al. 2015. PLoS Genet 11: e1005583), different organ biopsies (fetal n=26); GSE48472 (Slieker R C et al. 2013. Epigenetics Chromatin 6: 26), different organ biopsies (adults n=18); GSE58885 (Spiers H et al. 2015. Genome Res 25: 338-52), brain samples (fetal/embryonic n=179); and, GSE41826 (Guintivano J et al. 2013. Epigenetics 8: 290-302), frontal brain neurons (adult n=29).

### Example 7. Functional Annotation of Selection Regions

The regulatory features of candidate FCO loci were analyzed using ENCODE (Sloan C A et al. 2016. Nucleic Acids Res 44: D726-D732; Rosenbloom K R et al. 2013. Nucleic Acids Res 41: D56-63) and the functional features of the 27 candidates list were annotated using the human embryonic stem cells and human umbilical vein endothelial cell feature available therein.

### Example 8. Age Dependent Changes in the Fco Methylation Signature in Human Populations

The following example took advantage of several datasets with subjects of different ages. Five datasets were selected for this purpose: GSE83334 (Urdinguio R G et al. 2016. J Transl Med 14: 160), 15 paired samples (cord blood and five years old whole blood cells-WBC); GSE62219 (Acevedo N et al. 2015. Clin Epigenetics 7: 34), WBC samples from ten children; GSE36054 (Alisch R S et al, 2012. Genome Res 22: 623-632.), 176 WBC of children; and, GSE40279 (Hannum G et al. 2013. Mol Cell 49: 359-367), 656 adult WBC samples.

WBC and peripheral blood mononuclear cells samples available from the discovery and replication datasets were pooled (see Table 1).

### Example 9. Sensitivity Analyses

The method of Morin et al. (Morin A M et al. 2017. Clin Epigenetics 9: 75) was used to evaluate whether any of the UCB samples used in this manuscript showed evidence of maternal blood contamination. Ten CpGs were used to cluster the samples. UCB samples showing evident hypermethylation and with inconsistent DNA methylation age (>3.6 years margin of error reported by Horvath (Horvath 2013, herein)) were excluded from the analyses.

Maternal blood contamination in cord blood samples has been described (Morin et al. 2017, herein). Clearly maternal blood is a potential issue for contaminating cord blood in the present methods and processes. A signature of maternal blood contamination using ten probes from the 450K array was developed and validated using three pyrosequenced CpGs. Morin et al. used the Reinius et al. dataset (Reinius et al. 2012, herein) as an adult comparison and whole umbilical cord blood samples to detect differences in a linear model without further adjustment by age. A set of 2,250 CpGs was described as having potential targets for the differences between adult peripheral blood and cord blood based on mixed samples, rather than purified cells. A random forest approach was used to select a subset of highly hypomethylated ten CpGs in the cord blood, none of these CpGs were observed to be present within the FCO signature described herein. From this set of ten CpGs, a semi-quantitative index was developed, wherein if more than five CpGs out of ten demonstrated greater than a 20% difference in methylation, then that sample would qualify as being suspicious of maternal contamination. Although the filtering was based on a strict statistical rule, declaration of contamination mostly involved a qualitative assessment.

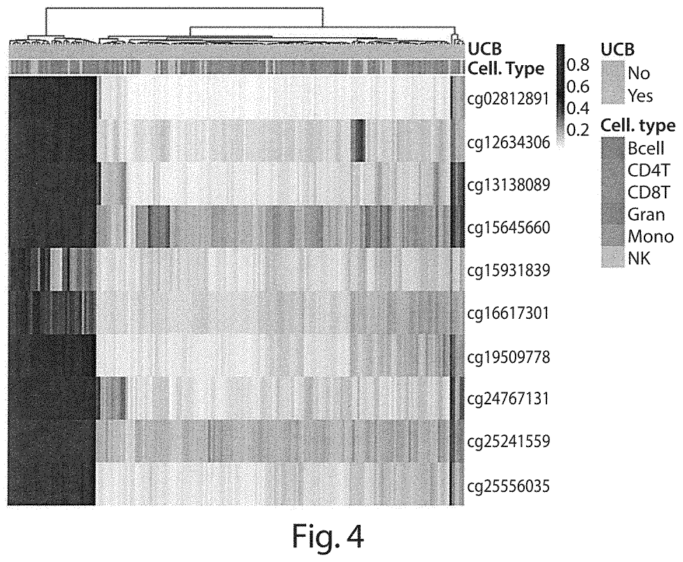

Accordingly, it was herein assessed whether any potential maternal contamination had occurred in the datasets using the method from Morin et al. Only one donor sample comprising all six isolated cells (indicated on the right side of the heatmap in FIG. 4) clustered slightly apart from the other samples. However, the DNA methylation age estimated for this sample (range: 0.82-2.95 years) was consistent with a UCB sample. It was also clarified that the DNA methylation age margin of error reported by Horvath was >3.6 years (Horvath 2013, herein). It was concluded herein that no evidence was obtained of significant contamination in the discovery data set used. Nonetheless, a sensitivity analysis was performed eliminating all six cells from that sample and stable results were observed.

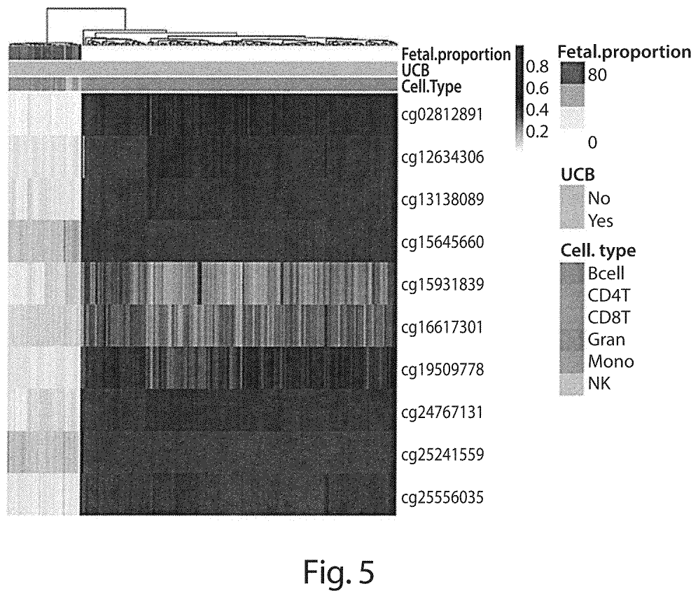

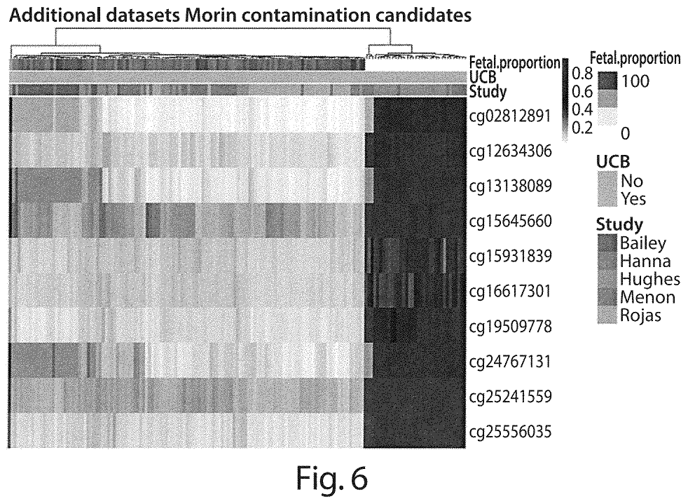

To further explore the idea of fetal contamination using the Morin makers the validation dataset was explored and the same results were achieved (FIG. 5). Therefore, the evidence from data and analyses herein does not support maternal contamination as a factor influencing the validity or interpretation of our cord blood samples or any of the other fetal and adult data. Five additional datasets used by Morin et al. were evaluated using the 10 CpGs in Morin et al., and one sample was observed herein among the new data that was clearly contaminated with maternal blood (FIG. 6). The contaminated sample was observed to cluster with adult blood and had an FCO signature of 0%, as observed in the heatmap in FIG. 6. In addition, the DNA methylation age of this sample was estimated 44.5 years in the “cord blood sample” vs 45 years in the maternal blood pair. As not all Morin et al. CpGs were present in the GEO datasets accessed, a K-nearest neighbors imputation was used to predict the 10 CpGs in cases where data were missing. This sample was therefore excluded from the analyses.

Taken together, these examples yielded confidence that maternal contamination is detected using a combination of the Morin et al. approach and the estimation of the DNA methylation age, should it exist, and that this factor can be ruled out as playing a significant role in final results.

### Example 10. Uses of the Methods

Several genome-scale DNA methylation data sets from newborn and adult leukocyte populations were used in examples herein to identify a common set of CpG loci among fetal leukocyte subtypes (the fetal cell origin, or FCO, signature) and applied to trace the proportion of cells with the progenitor phenotype in several tissue types across the lifecourse (Table 1). Without being limited by any particular theory or mechanism it is hypothesized herein that invariant methylation marks with high potential to be indicative of a FCO would be differentially methylated in newborns compared with adults and shared across six maj or blood cell lineages (granulocytes-Gran, monocytes-Mono, B lymphocytes-Bcell, CD4⁺ T lymphocytes-CD4T, CD8⁺ T lymphocytes-CD8T, and natural killer lymphocytes-NK).

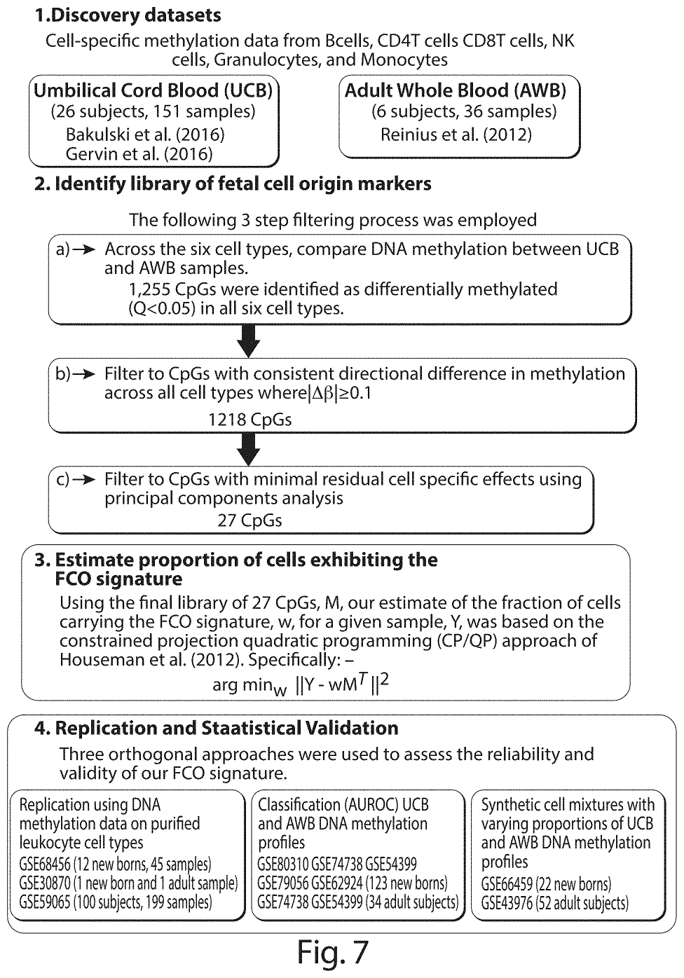

The analytic steps of the process for identification of candidate FCO CpGs from libraries of Illumina HumanMethylation450 array data are shown in FIG. 7. Genome-scale DNA methylation profiles of each of the six major blood cell lineages were initially compared separately between umbilical cord blood (UCB) and adult whole peripheral blood (AWB) DNA samples. Across the separate models fit to each blood cell type, 1,255 CpG sites were identified (False Discovery Rate, FDR<0.05) with shared, significant differential methylation between newborns and adults. Then, this lineage invariant subset of CpG loci was filtered to arrive at CpGs exhibiting both a consistent direction of differential methylation across all lineage groups and an absolute change in methylation greater than 10% between newborns and adults resulting in n=1218 CpGs associated with 518 genes.

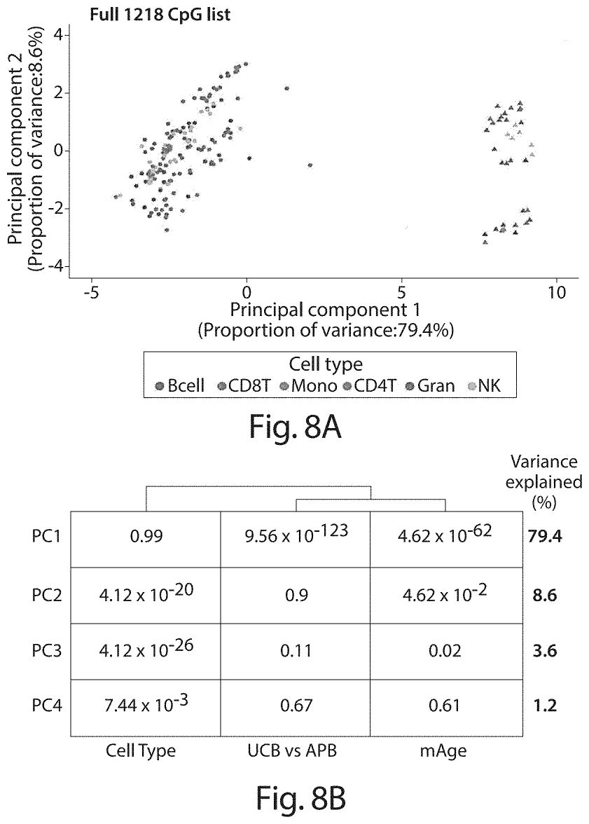

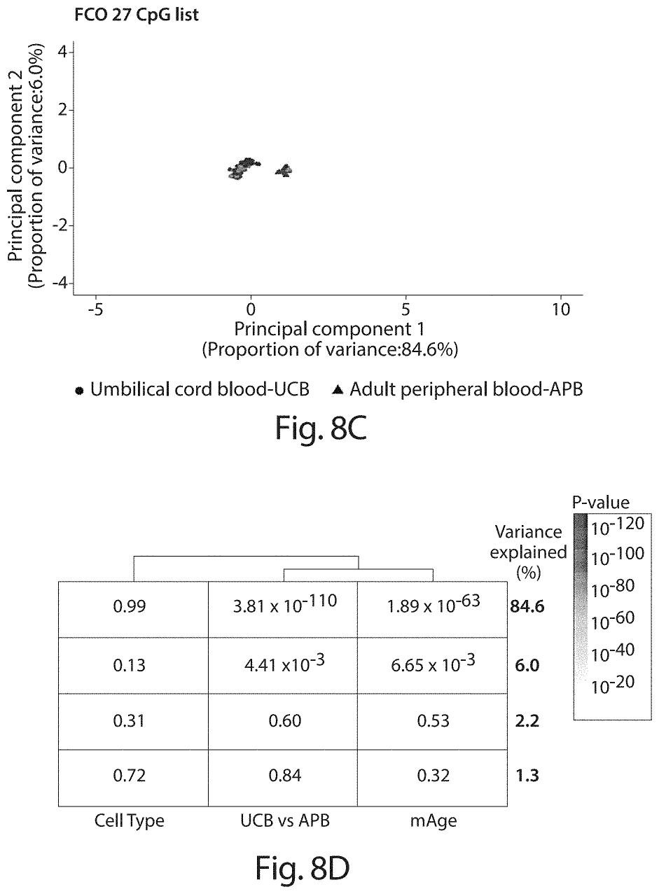

The list of candidate FCO CpG loci was further reduced (FIG. 8A) to minimize potential cell-type-specific contribution by selecting CpGs with minimal residual cell-specific effects, resulting in 27 CpGs (FIG. 8B). This was accomplished by using a principal component regression analysis in which the standardized, and rotated scores of the first four principal components captured most of the variation in DNA methylation across the 1,218 candidate CpGs. The first principal component explained 79.4% of the variance and was significantly associated with both methylation age (P=4.62×10⁻⁶²) and UCB vs adult peripheral blood (P=9.56×10⁻¹²³). Some residual variability, 13.4%, was significantly associated with cell type in the second to fourth principal components (FIG. 8A, lower heatmap). Once filtered to 27 CpGs, 84.6% of the variance was explained by the first principal component, which was significantly associated with both methylation age (P=1.89×10⁻⁶³) and UCB compared to adult peripheral blood (P=3.81×10⁻¹¹⁰). However, cell type was no longer significantly associated with any of the first four principal components (94.1% of the total variance, FIG. 8B, lower heatmap). The library of 27 CpGs so identified represents a phenotypic block of differentially methylated regions (DMRs), with a fetal cell origin phenotype here defined as the FCO signature. The term “FCO signature” summarizes the idea of a common invariant biomarker of a cell that originated during the prenatal period, which is also present across different cell lineage subtypes but which is reduced or lost during lineage commitment of progenitor cells in the adult.

The FCO library was then used in conjunction with the constrained projection quadratic programming approach of Houseman et al. (Houseman et al. 2012, herein; Koestler et al. 2016, herein; Accomando et al. 2014, herein), to estimate the proportion of cells exhibiting the FCO signature in a manner agnostic to variation in underlying proportions of cell types in any given sample, and independent of a sample's DNA methylation age (Horvath 2013, herein; Hannum et al. 2013, herein). The proportion of cells with the FCO signature was estimated for each sample in the discovery data set of newborn and adult leukocytes. UCB samples were predicted to harbor a very high proportion of cells of fetal origin (mean=85.4%), significantly higher than adult leukocytes (mean=0.6%, P=2.11×10⁻¹⁹¹, FIG. 1A). To replicate these findings, the same estimation approach was applied to an independent data set that included leukocyte-specific methylation measurements collected from newborn and adult sources. In the replication data set similar differences were observed in proportions of cells with the stem cell lineage signature between cord blood and adults (P=8.35×10⁻⁸¹, FIG. 1B), where the proportion of cells exhibiting the FCO signature was higher in the cord blood samples compared to the adult samples (89.9% versus 2.0% for UCB and AWB samples, respectively). Together, these results show that the FCO signature captures a population of lineage invariant, developmentally sensitive cells.

Once concordant results in the validation data were obtained the classification performance of the 27 CpG in the FCO signature compared to randomly selected sets of CpGs was assessed. Five independent data sets were included (Table 1, AUROC datasets) consisting of n=123 umbilical cord blood and n=34 adult whole peripheral blood samples. As Morin et al. 2017, herein had interrogated the potential of maternal blood contamination using these datasets, evident maternal blood contamination in any of the samples was located. Using a combination of the 10 CpGs reported by Morin et al. and the calculation of DNA methylation age, one cord blood sample was found in the paired maternal-newborn GSE54399 dataset (Montoya-Williams et al. 2017, herein) that was mostly maternal blood (DNA methylation age 44.5 years corresponding to the paired 45 years in the maternal sample and an adult hypermethylated pattern using the ten markers of Morin et al. 2017, herein). After removing this sample, the FCO signature was applied to these data, to assess how well the FCO signature classified fetal from adult tissues by computing the area under the receiver operating characteristic curve (AUROC). The AUROC for the 27 CpG FCO signature was estimated to be 0.996 based on a combined analysis of the five data sets described above. To gauge whether the AUROC was statistically significant, and thus, that the 27 CpG FCO signature represents a statistically significant subset, an analysis was conducted in which the empirical null distribution of the AUROC was generated by randomly selecting subsets of CpGs of size 27, followed by calculation of the AUROC for the randomly selected subset. These steps were repeated 10,000 times to compute the probability of observing an AUROC as large or larger than what was computed based on our 27 CpG FCO signature. The P from this randomization-based test was P=0.0193, meaning that there was only a 1.9% chance of observing an AUROC as large or larger than what was observed based on the FCO signature. In addition, this same dataset was used to evaluate how stable the estimations would be if some of the 27 markers were excluded using a leave one out combination, leave two out combination, until five probes combination were removed. Although the estimates were stable in the absence of several of the probes, the potential error increases per probe removed (average RMSE: 10 when removing one probe, 15 when removing two, 19 when removing three, 22 with four and 25 with five).

To establish the stability of the FCO signature, the absolute difference in the FCO estimates was evaluated when all the potential combinations of one to five CpGs were lost during the FCO estimations compared to the full set of 27 CpGs using the samples used for the AUROC analysis (umbilical cord blood GSE80310 (Knight et al. 2016, herein), GSE74738 (Hanna et al. 2016, herein), GSE54399 (Montoya-Williams et al. 2017, herein), GSE79056 (Knight et al. 2016, herein), GSE62924 (Rojas et al. 2015, herein). Adult peripheral blood GSE74738 (Hanna et al. 2016, herein), GSE54399 (Montoya-Williams et al. 2017, herein). The average root mean square error (RMSE) between the prediction using the 27 CpGs vs all the potential combinations was also calculated when as few as one CpG and as many as five CpGs were excluded from the 27 FCO CpGs. The results indicate that the 27 CpG sites is a minimum discriminatory set for a reliable FCO estimation.

Within the 27 CpGs the loss of eight probes (cg01278041, cg05840541, cg11194994, cg11199014, cg13485366, cg14652587, cg17471939, cg22497969) had the biggest impact in the FCO calculations (RMSE>10). In contrast the loss of some other probes (e.g. cg01567783, absent in the EPIC array), only altered minimally the FCO estimates (RMSE:2.24). It is recommended that the full set of probes be used for the calculations but in the absence of specific probes the user of the methods should consider the increase in the estimation errors.

To further demonstrate the validity and reliability of the signature, reference synthetic cell mixtures were generated by mixing cord-blood and adult peripheral blood DNA methylation signatures in silico (Table 1, synthetic mixtures datasets), varying the fraction of fetal cord-blood across mixtures. Application of the method to the reference synthetic cell mixtures showed a high concordance correlation coefficient between the estimated fraction of cells carrying the FCO signature and the known mixture proportions (FIG. 9A, concordance correlation coefficient, CCC=0.97).

To explore the ontogeny of the FCO signature, the methylation array data were deconvoluted from each of embryonal stem cell lines, induced pluripotent cells (iPCS), fetal CD34⁺ stem/progenitor cells and bone marrow adult CD34⁺ stem/progenitor cells. The results indicated concordance of the leukocyte derived FCO signature with embryonal and pluripotent methylomes (Table 2 and FIG. 10). Surprisingly, the data showed the fact that among the ESC and iPCS, there was a wide range of the estimated FCO signature. Using information on the number of passages (subcultures) per sample (mean=27.2 passages, SD: 16.8), the estimated FCO fraction was modeled against the number of cell culture passages using a linear regression model. For every additional passage, a reduction of 0.14%, on average, was observed in the estimated FCO signature (P=0.01) after adjusting for each sample's estimated DNA methylation age, FIG. 11. This trend was observed in both ESC and iPSC, however, when stratifying by cell type the magnitude of the reduction was higher for ESC (a mean reduction of 0.18% per passage), and it was attenuated in the iPSCs (a reduction of 0.07% per passage), the P of interaction for cell passage and cell type was not statistically significant P=0.11.

A potential caveat for deriving the FCO signature is the use of lineage committed neonatal cord and adult peripheral blood cells rather than the use of undifferentiated fetal and adult progenitor cells. One reason for this is the fact that considerable heterogeneity exists in isolating undifferentiated cells, making it problematic to generate a true “gold standard”. As an approximation and to estimate the relative variability and sources of uncertainty of our FCO signature we applied a similar pipeline and filter criteria to a small dataset of fetal and adult pluripotent cells. In this sensitivity analysis the DNA methylation was compared between 19 undifferentiated ESCs and five adult hematopoietic stem cells (CD34⁺ CD38⁻ CD90⁺ CD45RA⁻) as proxies of common pluripotent cells at the embryonic and adult ages, respectively. Of observed 113 differentially methylated sites (FDR<0.05) that overlapped with the original 1,255 candidate list (9% overlap) generated from differentiated cells, five out of the 27 CpGs (19%) in the FCO signature were represented. However, when the same filtering process was applied to those CpGs to remove lineage specific effects (see methods), only two CpGs out of the 113 CpGs were retained. When the 113 overlapping CpGs were explored using the discovery dataset, cell population stratification was observed. The second principal component variance increased from 6.0% using the 27 CpGs (FIG. 8B) to 9.8% using the 113 CpGs, and in contrast to the approach as applied to differentiated blood cells, these 113 CpGs discriminated myeloid and lymphoid subpopulations in both the fetal and adult cells of the discovery dataset. The distribution and the variance explained resembled the distribution observed using the 1218 CpGs from the candidate list (FIG. 8A). This finding indicates a highly heterogeneous ESC population in this small sensitivity analysis, which is also consistent with the observed variance in FCO fraction of ESCs explained by cell culture passage number. However, these results also show that the FCO signature shares some CpG loci in common with those derived from a pipeline that starts with ESCs and adult progenitors.

It was then reasoned that if part of the FCO signature were an indicator of embryonic stem cell lineage, it would also be detectable among non-hematopoietic fetal tissues. FIG. 12A shows the high FCO fraction in diverse fetal tissues (3 to 26 weeks of gestational age) and in sharp contrast, the minimal representation of the FCO signature in adult tissues. The FCO signature demonstrated higher variability in fetal/embryonic brain and muscle, showing a dramatic drop of the signature with later gestational age, FIG. 12B, compared to other tissues including the liver (a hematopoietic tissue in the fetus).

The potential biologic functions of the FCO signature were explored. To include sufficient genes in this analysis, analysis was returned to the filtered lineage invariant fetal cell origin candidate CpG list (n=1218 CpGs, associated with 518 genes), and a test of enrichment was applied using information from the MsigDB curated databases v. 6.0 (Liberzon A et al. 2011. Bioinformatics 27: 1739-40) and the Progenitor Cell Biology Consortium database (Salomonis et al. 2016, herein). Several methodological approaches were used to test for enrichment using the curated molecular signatures database (MSigDB): ToppGene (Chen J et al. 2009. Nucleic Acids Res 37: 305-311), GREAT (McLean et al. 2010, herein), and missMethyl (Phipson et al. 2016, herein). ToppGene and missMethyl used the 518 genes associated with the CpG site, in contrast, GREAT used 1238 genes within 1 Mb of the CpG site (cis-regulatory genes). In total 18, 20 and 27 pathways were statistically significant after FDR correction respectively. Of those, a significant statistical association was found in nine pathways using the three approaches, and in six pathways overlapping the ToppGene and missMethyl approaches (shown in Table 3 which is a functional annotation of the 27 loci included in the ESC methylation signature).

TABLE 3
MSigDB pathways test for enrichment with DMRs contained in lineage invariant developmentally sensitive loci (N = 1218)

| | | | | | |
|---|---|---|---|---|---|
| | | | | | DM |
| ID | MSigDB Pathways | Cell target of the pathway | K | DM | (cis) |

Genes identified by ChIP on chip as targets of a Polycomb protein or Polycomb
Repression Complex 2 (bound to protein and H3K27 tri-methylation (H3K27me3))

| | | | | | |
|---|---|---|---|---|---|
| M9898 | BENPORATH_SUZ12_TARGETS | Human embryonic stem cells | 1038 | 112 | 183 |
| M7617 | BENPORATH_EED_TARGETS | Human embryonic stem cells | 1062 | 105 | 184 |
| M8448 | BENPORATH_PRC2_TARGETS | Human embryonic stem cells | 652 | 83 | 138 |

Genes with high-CpG-density promoters (HCP) bearing the H3K27 tri-methylation (H3K27me3)

| | | | | | |
|---|---|---|---|---|---|
| M10371 | BENPORATH_ES_WITH_H3K27ME3 | Human embryonic stem cells | 1118 | 122 | 210 |
| M1938 | MEISSNER_BRAIN_HCP_WITH_H3K27ME3 | Brain | 269 | 39 | 80 |
| M1967 | MIKKELSEN_IPS_WITH_HCP_H3K27ME3 | MCV8.1 (induced pluripotent | 102 | 22 | 28 |
| | | cells, iPS) | | | |
| M2009 | MIKKELSEN_NPC_HCP_WITH_H3K27ME3 | Neural progenitor cells (NPC) | 341 | 39 | 78 |
| M1932 | MEISSNER_NPC_HCP_WITH_H3K27ME3 | Neural precursor cells (NPC) | 79 | 12 | 22 |
| M1954 | MIKKELSEN_MCV6_HCP_WITH_H3K27ME3 | MCV6 cells (embryonic | 435 | 43 | |
| | | fibroblasts trapped in a | | | |
| | | differentiated state) | | | |
| M2019 | MIKKELSEN_MEF_HCP_WITH_H3K27ME3 | MEF cells (embryonic | 590 | 48 | |
| | | fibroblast) | | | |

Genes with high-CpG-density promoters (HCP) that have no H3K27 tri-methylation (H3K27me3)

| | | | | | |
|---|---|---|---|---|---|
| M1936 | MEISSNER_NPC_HCP_WITH_H3_UNMETHYLATED | Neural precursor cells (NPC) | 536 | 44 | 65 |

Genes with high-CpG-density promoters (HCP) bearing histone H3
dimethylation at K4 (H3K4me2) and trimethylation at K27 (H3K27me3)

| | | | | |
|---|---|---|---|---|
| M1941 | MEISSNER_BRAIN_HCP_WITH_H3K4ME3_AND_H3K27ME3 | Brain | 1069 | 83 |
| M1949 | MEISSNER_NPC_HCP_WIIH_H3K4ME2_AND_H3K27ME3 | Neural precursor cells (NPC) | 349 | 34 |

Genes hypermethylated in tumor cells

| | | | | |
|---|---|---|---|---|
| M19508 | HATADA_METHYLATED_IN_LUNG_CANCER_UP | Lung cancer cells | 390 | 32 |

Genes up-regulated in tumor cells

| | | | | |
|---|---|---|---|---|
| M2098 | MARIENS_TRETINOIN_RESPONSE_UP | NB4 cells (acute | 857 | 50 |
| | | promyelocytic leukemia, | | |
| | | APL) | | |

| | ToppGene | | GREAT | | | missMethyl | |
|---|---|---|---|---|---|---|---|
| ID | P | FDR | FE | P | FDR | P | FDR |
| Genes identified by ChIP on chip as targets of a Polycomb protein or Polycomb | | | | | | | |
| Repression Complex 2 (bound to protein and H3K27 tri-methylation (H3K27me3)) | | | | | | | |
| M9898 | 2.86 × 10⁻⁴¹ | 1.33 × 10⁻³⁷ | 2.09 | 1.92 × 10⁻³⁸ | 1.61 × 10⁻³⁵ | <2.0 × 10⁻¹⁶ | <2.0 × 10⁻¹⁶ |
| M7617 | 6.79 × 10⁻³⁶ | 1.58 × 10⁻³² | 2.06 | 2.68 × 10⁻³⁷ | 1.80 × 10⁻³⁴ | <2.0 × 10⁻¹⁶ | <2.0 × 10⁻¹⁶ |
| M8448 | 3.49 × 10⁻³⁶ | 1.08 × 10⁻³² | 2.59 | 4.19 × 10⁻⁴⁶ | 4.69 × 10⁻⁴³ | <2.0 × 10⁻¹⁶ | <2.0 × 10⁻¹⁶ |
| Genes with high-CpG-density promoters (HCP) bearing the H3K27 tri-methylation (H3K27me3) | | | | | | | |
| M10371 | 1.48 × 10⁻⁴⁶ | 1.38 × 10⁻⁴² | 2.18 | 4.47 × 10⁻⁵⁰ | 7.51 × 10⁻⁴⁷ | <2.0 × 10⁻¹⁶ | <2.0 × 10⁻¹⁶ |
| M1938 | 2.16 × 10⁻¹⁹ | 3.36 × 10⁻¹⁶ | 3.71 | 1.31 × 10⁻⁵¹ | 4.40 × 10⁻⁴⁸ | 2.90 × 10⁻¹² | 2.74 × 10⁻⁹ |
| M1967 | 3.53 × 10⁻¹⁵ | 4.11 × 10⁻¹² | 4.99 | 7.61 × 10⁻³⁶ | 4.27 × 10⁻³³ | 8.32 × 10⁻¹⁰ | 6.55 × 10⁻⁷ |
| M2009 | 8.50 × 10⁻¹⁶ | 1.13 × 10⁻¹² | 2.38 | 2.12 × 10⁻²¹ | 1.02 × 10⁻¹⁸ | 1.97 × 10⁻⁸ | 1.17 × 10⁻⁵ |
| M1932 | 4.13 × 10⁻⁷ | 1.60 × 10⁻⁴ | 3.50 | 8.53 × 10⁻¹⁵ | 2.61 × 10⁻¹² | 3.07 × 10⁻⁵ | 9.06 × 10⁻³ |
| M1954 | 5.14 × 10⁻¹² | 5.00 × 10⁻¹¹ | | | N.S | 1.96 × 10⁻⁷ | 9.27 × 10⁻⁵ |
| M2019 | 6.86 × 10⁻¹⁰ | 6.66 × 10⁻⁹ | | | N.S | 2 × 10⁻⁶ | 8.47 × 10⁻⁴ |
| Genes with high-CpG-density promoters (HCP) that have no H3K27 tri-methylation (H3K27me3) | | | | | | | |
| M1936 | 1.65 × 10⁻¹² | 1.18 × 10⁻⁹ | 2.06 | 1.69 × 10⁻¹⁴ | 4.36 × 10⁻¹² | 3.4 × 10⁻⁸ | 1.79 × 10⁻⁵ |
| Genes with high-CpG-density promoters (HCP) bearing histone H3 | | | | | | | |
| dimethylation at K4 (H3K4me2) and trimethylation at K27 (H3K27me3) | | | | | | | |
| M1941 | 5.42 × 10⁻¹⁸ | 5.26 × 10⁻¹⁷ | | | N.S | 1.86 × 10⁻⁸ | 1.17 × 10⁻⁵ |
| M1949 | 3.85 × 10⁻⁹ | 3.74 × 10⁻⁸ | | | N.S | 9.3 × 10⁻⁶ | 3.38 × 10⁻³ |
| Genes hypermethylated in tumor cells | | | | | | | |
| M19508 | 4.05 × 10⁻⁶ | 3.93 × 10⁻⁵ | | | N.S | 2.5 × 10⁻⁵ | 7.97 × 10⁻³ |
| Genes up-regulated in tumor cells | | | | | | | |
| M2098 | 1.17 × 10⁻⁵ | 1.14 × 10⁻⁴ | | | N.S | 3.5 × 10⁻⁶ | 1.36 × 10⁻³ |

Note:
the table summarizes only the significant pathways overlapping three different methods to test for enrichment: 1) ToppGene, hypergeometric distribution to test for enrichment, 2) GREAT, binomial test to test for enrichment cis-regulatory regions, and 3) missMethyl which allows adjusting for array bias.
Abbreviations: ID (MSigDB internal identifier), K (number of genes contained in the gene set), DM (differentially methylated genes overlapping the CpG site), DM (cis) (cis-regulatory regions either overlapping the differentially methylated CpG site or 1 Mb around the site), P (unadjusted P-value), FDR (False discovery), FE (Fold enrichment), N.S (not significant association, FDR > 0.05)

Among the nine overlapping the three approaches, there was a statistically significant association with pathways related to epigenetic marks in embryonal stem cells and progenitor cells. When restricting to the FCO signature CpGs there was an interesting pattern in the chromatin features of 11 out of the 27 sites that changed from a poised promoter to a repressed state in umbilical vein endothelial cells (Table 4).

Table 4 is a list of transcription factors with DMRs contained in lineage invariant developmentally sensitive loci (N=834).

TABLE 4
Functional annotation using ENCODE data of the loci
included in the FCO methylation signature

| | Human Embryonic | Human umbilical vein | Transcription | Transcription |
|---|---|---|---|---|
| Probe ID | Stem cell | endothelial cell | factor 1 | factor 2 |
| cg10338787 | 3_Poised_Promoter | 12_Repressed | EZH2 | EZH2 |
| cg22497969 | 13_Heterochromatin/ | 13_Heterochromatin/ | | |
| | low signal | low signal | | |
| cg11968804 | 3_Poised_Promoter | 12_Repressed | | |
| cg10237252 | 6_Weak_Enhancer | 12_Repressed | Pol2 | |
| cg17310258 | 3_Poised_Promoter | 12_Repressed | EZH2 | EZH2 |
| cg13485366 | 13_Heterochromatin/ | 13_Heterochromatin/ | | |
| | low signal | low signal | | |
| cg03455765 | 2_Weak_Promoter | 12_Repressed | | |
| cg04193160 | 3_Poised_Promoter | 12_Repressed | USF-1 | Bachl |
| cg27367526 | 2_Weak_Promoter | 1_Active Promoter | | |
| cg03384000 | 3_Poised_Promoter | 1_Active Promoter | SIN3A | |
| cg15575683 | 3_Poised_Promoter | 12_Repressed | YY1 | |
| cg17471939 | 3_Poised_Promoter | 13_Heterochromatin/ | | |
| | | low signal | | |
| cg11199014 | 3_Poised_Promoter | 3_Poised Promoter | Pol2 | RBBP5 |
| cg13948430 | 3_Poised_Promoter | 12_Repressed | | |
| cg01567783 | 3_Poised_Promoter | 12_Repressed | | |
| cg01278041 | 2_Weak_Promoter | 11_Weak_Transcribed | CHD1 | TAF1 |
| cg19005955 | 7_Weak_Enhancer | 4_Strong_Enhancer | | |
| cg16154155 | 3_Poised_Promoter | 12_Repressed | EZH2 | EZH2 |
| cg14652587 | 3_Poised_Promoter | 12_Repressed | | |
| cg19659741 | 6_Weak_Enhancer | 12_Repressed | | |
| cg06705930 | 3_Poised_Promoter | 12_Repressed | SUZ12 | |
| cg23009780 | 5_Strong_Enhancer | 12_Repressed | | |
| cg22130008 | 3_Poised_Promoter | 3_Poised Promoter | | |
| cg05840541 | 13_Heterochromatin/ | 13_Heterochromatin/ | | |
| | low signal | low signal | | |
| cg06953130 | 2_Weak_Promoter | 5_Strong_Enhancer | | |
| cg11194994 | 2_Weak_Promoter | 4_Strong_Enhancer | | |
| cg14375747 | 6_Weak_Enhancer | 12_Repressed | TBP | |

In addition, among the candidate stem cell gene list were 13 homeobox transcription factors as well as 14 others that play key roles in embryonic development (e.g. FOXD2, FOXE3, FOXI2, FOXL2, ARID3A, NFIX, PRDM16, SOX18, Table 5).

Table 5 shows MDSigDB pathways enriched with DMRs contained in lineage invariant developmentally sensitive loci (N=834).

TABLE 5
Transcription factors with DMRs contained in lineage
invariant developmentally sensitive loci (N = 1218).

| | |
|---|---|
| Transcription factor | Name |

Zinc-coordinating DNA-binding domains

| KLF9 | Kruppel Like Factor 9 |
|---|---|
| ZBTB46 | Zinc Finger BTB |
| | Domain Containing 46 |
| PRDM10 | PR/SET Domain 10 |
| PRDM16 | PR/SET Domain 12 |
| Helix-turn-helix domains | |
| Homeo domain factors | |
| HOXA2 | Homeobox A2 |
| HOXB7 | Homeobox B7 |
| HOXB-AS3 | HOXB Cluster Antisense RNA 3 |
| LBX2 | Ladybird Homeobox 2 |
| VAX2 | Ventral Anterior Homeobox 2 |
| ALX4 | ALX Homeobox 4 |
| PITX3 | Paired Like Homeodomain 3 |
| LHX6 | LIM Homeobox 6 |
| SIX2 | SIX homeobox 2 |
| POU2F1 (Oct. 1) | POU Class 2 Factor 1 |
| POU3F1 (Oct. 6) | POU Class 3 Homeobox 1 |

Paired box factors

| | |
|---|---|
| PAX6 | Homeodomain Paired box 6 |
| PAX8 | Homeodomain Paired box 8 |
| FOXE3 | Forkhead binding E3 |
| FOXD2 | Forkhead binding D2 |
| FOXI2 | Forkhead binding 12 |
| FOXL2 | Forkhead binding L2 |
| FOXL2NB | FOXL2 Neighbor |

Tryptophan cluster factors

| | |
|---|---|
| ETV4 | ETS variant 4 |

ARID

| | |
|---|---|
| ARID3A | AT-Rich Interaction Domain 3A |

Other all-α-helical DNA-binding domains

| | |
|---|---|
| SOX18 | SRY-Box 18 |

Immunoglobulin fold

| | |
|---|---|
| TBX1 | T-Box 1 |
| TBX4 | T-Box 4 |

β-Hairpin e×posed by an α/β-scaffold

| | |
|---|---|
| NF-1X | Nuclear Factor 1 X |

Most notable were genes previously implicated in fetal to adult transitions in hematopoiesis. ARID3A plays a critical role in lineage commitment in early hematopoiesis (Ratliff et al. 2014, herein). Among the targets was SOX18, a paralog of SOX17, the latter being shown to maintain fetal characteristics of HSCs in mice (He et al. 2011). PRC2 targets were overrepresented in FCO signature loci (Table 3 and Table 4). EZH2, one of three PRC2 components, is indispensable for fetal liver hematopoiesis, but largely dispensable for adult bone marrow hematopoiesis (Mochizuki-Kashio et al. 2011, herein; Xie et al. 2014, herein; Oshima et al. 2016, herein). Among the larger set of loci used to derive the FCO signature, there are five DMRs within the MIIRLET7BHG locus (FIG. 13). The LIN28A-LIN28BAlet-7 axis is a highly evolutionarily conserved developmental regulator and has emerged as a prominent feature of the fetal to adult switch in murine hematopoiesis (Copley M R et al. 2013. Nat Cell Biol 15: 916-25; Rowe et al. 2016, herein). The DMR region identified herein encompasses exon and intron 1 of the MIRLET7BHG. Methylation in this region displayed an inverse relationship within fetal and adult cells for CpG boundary probes that co-locate with active histone marks, DNase I hypersensitivity and transcription factor binding sites (FIG. 13). In addition, a middle region, which is devoid of regulatory motifs, displayed contrasting methylation pattern with hypomethylated loci in adult cells demarcated by hypermethylation, whereas in embryonic cells, the bipartite region is bounded by hypermethylated loci demarcated by hypomethylation. In addition, over representation of genes expressed in ESC to embryoid body differentiation were among the FCO methylation gene loci (Table 6).

Table 6 shows progenitor cell biology consortium (PCBC) pathways enriched using Toppgene with DMRs contained in lineage invariant developmentally sensitive loci (N=834).

TABLE 6
Progenitor Cell Biology Consortium (PCBC) pathways test for enrichment using ToppGene
with DMRs contained in lineage invariant developmentally sensitive loci (N = 1218).

| | | | | |
|---|---|---|---|---|
| | # Genes in | | | |
| | GeneSet | | | |
| PCBC Pathway | (K) | DM | P | FDR |

Stem cells top expressed genes

| | | | | |
|---|---|---|---|---|
| Arv_EB-LF_2500_K2 | 960 | 59 | 3.21 × 10⁻¹⁰ | 1.04 × 10⁻⁸ |
| Arv_EB-LF_1000 | 990 | 58 | 2.73 × 10⁻⁹ | 7.62 × 10⁻⁸ |
| Arv_EB-LF_1000_K4 | 436 | 33 | 2.67 × 10⁻⁸ | 5.66 × 10⁻⁷ |
| Arv_EB-LF_500_K2 | 256 | 23 | 1.77 × 10⁻⁷ | 3.11 × 10⁻⁶ |
| PCBC_SC_CD34+_1000 | 987 | 53 | 2.33 × 10⁻⁷ | 3.77 × 10⁻⁶ |
| Arv_EB-LF_500 | 499 | 32 | 1.75 × 10⁻⁶ | 2.45 × 10⁻⁵ |
| Arv_SC-LF_1000_K3 | 679 | 39 | 2.01 × 10⁻⁶ | 2.74 × 10⁻⁵ |

Embryoid body vs Stem Cells

| | | | | |
|---|---|---|---|---|
| PCBC_ratio_EB_vs_SC_1000 | 997 | 86 | 8.85 × 10⁻²⁴ | 5.43 × 10⁻²¹ |
| ratio_EB_vs_SC_2500_K3 | 1102 | 79 | 4.62 × 10⁻¹⁷ | 9.46 × 10⁻¹⁵ |
| PCBC_ratio_EB_vs_SC_500 | 499 | 47 | 1.01 × 10⁻¹⁴ | 1.03 × 10⁻¹² |
| ratio_EB_vs_SC_1000_K5 | 418 | 42 | 3.14 × 10⁻¹⁴ | 2.75 × 10⁻¹² |
| ratio_EB_vs_SC_1000_K1 | 336 | 29 | 1.09 × 10⁻⁸ | 2.67 × 10⁻⁷ |
| ratio_EB_vs_SC_500_K3 | 204 | 22 | 1.26 × 10⁻⁸ | 2.98 × 10⁻⁷ |

Ectoderm vs Stem cell

| | | | | |
|---|---|---|---|---|
| ratio_ECTO_vs_SC_2500_K3 | 854 | 60 | 9.51 × 10⁻¹³ | 5.84 × 10⁻¹¹ |
| ratio_ECTO_vs_SC_500_K1 | 283 | 32 | 1.67 × 10⁻¹² | 9.34 × 10⁻¹¹ |
| ratio_ECTO_vs_SC_1000_K3 | 476 | 42 | 2.47 × 10⁻¹² | 1.26 × 10⁻¹⁰ |
| PCBC_ratio_ECTO_vs_SC_500 | 499 | 42 | 1.14 × 10⁻¹¹ | 5.01 × 10⁻¹⁰ |
| PCBC_ratio_ECTO_vs_SC_1000 | 994 | 61 | 1.65 × 10⁻¹⁰ | 5.64 × 10⁻⁹ |
| PCBC_ratio_ECTO_vs_SC_100 | 100 | 14 | 2.32 × 10⁻⁷ | 3.77 × 10⁻⁶ |

Endoderm vs Stem cell

| | | | | |
|---|---|---|---|---|
| PCBC_ratio_DE_vs_SC_500 | 499 | 36 | 2.13 × 10⁻⁸ | 4.66 × 10⁻⁷ |
| ratio_DE_vs_SC_500_K5 | 300 | 26 | 5.79 × 10⁻⁸ | 1.15 × 10⁻⁶ |
| ratio_DE_vs_SC_500_K1 | 377 | 29 | 1.34 × 10⁻⁷ | 2.50 × 10⁻⁶ |
| ratio_DE_vs_SC_1000_K5 | 542 | 36 | 1.68 × 10⁻⁷ | 3.03 × 10⁻⁶ |
| PCBC_ratio_DE_vs_SC_1000 | 998 | 49 | 8.25 × 10⁻⁶ | 1.01 × 10⁻⁴ |
| ratio_DE_vs_SC_1000_K2 | 523 | 31 | 1.24 × 10⁻⁵ | 1.43 × 10⁻⁴ |

Mesoderm vs Stem cell

| | | | | |
|---|---|---|---|---|
| PCBC_ratio_MESO- | 499 | 34 | 2.06 × 10⁻⁷ | 3.51 × 10⁻⁶ |
| 5_vs_SC_500 | | | | |
| PCBC_ratio_MESO- | 994 | 51 | 1.53 × 10⁻⁶ | 2.24 × 10⁻⁵ |
| 5_vs_SC_1000 | | | | |
| ratio_MESO_vs_SC_500_K1 | 297 | 22 | 8.01 × 10⁻⁶ | 1.00 × 10⁻⁴ |

Embryoid body top expressed genes

| | | | | |
|---|---|---|---|---|
| PCBC_EB_1000 | 997 | 81 | 9.22 × 10⁻²¹ | 2.83 × 10⁻¹⁸ |
| PCBC_EB_500 | 499 | 45 | 1.82 × 10⁻¹³ | 1.40 × 10⁻¹¹ |

Embryoid body vs non-stem cells

| | | | | |
|---|---|---|---|---|
| PCBC_EB_blastocyst_1000 | 995 | 74 | 7.21 × 10⁻¹⁷ | 1.11 × 10⁻¹⁴ |
| PCBC_EB_fibroblast_1000 | 992 | 71 | 2.38 × 10⁻¹⁵ | 2.93 × 10⁻¹³ |
| PCBC_EB_fibrob1ast_500 | 499 | 44 | 7.42 × 10⁻¹³ | 5.06 × 10⁻¹¹ |
| PCBC_EB_blastocyst_500 | 498 | 41 | 4.04 × 10⁻¹¹ | 1.55 × 10⁻⁹ |

Ectoderm top expressed genes

| | | | | |
|---|---|---|---|---|
| PCBC_ECTO_fibrob1ast_1000 | 996 | 62 | 6.46 × 10⁻¹¹ | 2.33 × 10⁻⁹ |
| PCBC_ECTO_fibrob1ast_500 | 499 | 39 | 5.61 × 10⁻¹⁰ | 1.72 × 10⁻⁸ |
| PCBC_ECTO_500 | 498 | 37 | 6.18 × 10⁻⁹ | 1.65 × 10⁻⁷ |
| PCBC_ECTO_1000 | 997 | 57 | 9.06 × 10⁻⁹ | 2.32 × 10⁻⁷ |
| PCBC_ECTO_blastocyst_1000 | 986 | 56 | 1.55 × 10⁻⁸ | 3.53 × 10⁻⁷ |
| PCBC_ECTO_blastocyst_500 | 490 | 34 | 1.34 × 10⁻⁷ | 2.50 × 10⁻⁶ |

Mesoderm top expressed genes

| | | | | |
|---|---|---|---|---|
| PCBC_MESO-5_blastocyst_1000 | 979 | 52 | 4.26 × 10⁻⁷ | 6.71 × 10⁻⁶ |
| PCBC_MESO-5_fibroblast_1000 | 985 | 50 | 2.64 × 10⁻⁶ | 3.53 × 10⁻⁵ |
| PCBC_MESO-5_500 | 494 | 30 | 1.08 × 10⁻⁵ | 1.29 × 10⁻⁴ |

Other differentiated cells

| | | | | |
|---|---|---|---|---|
| JC_fibro_1000 | 994 | 64 | 7.28 × 10⁻¹² | 3.44 × 10⁻¹⁰ |
| geo_heart_1000_K5 | 428 | 38 | 2.36 × 10⁻¹¹ | 9.67 × 10⁻¹⁰ |
| JC_fibro_500 | 497 | 38 | 1.74 × 10⁻⁹ | 5.08 × 10⁻⁸ |
| PCBC_ctl_geo-heart_1000 | 997 | 55 | 5.60 × 10⁻⁸ | 1.15 × 10⁻⁶ |
| JC_fibro_2500_K5 | 826 | 43 | 7.36 × 10⁻⁶ | 9.42 × 10⁻⁵ |
| JC_fibro_1000_K4 | 177 | 16 | 1.22 × 10⁻⁵ | 1.43 × 10⁻⁴ |

Taken together, the examples herein provide a deconvolution method based on DNA methylation that indicates the fraction of differentiated cells with fetal cell origins which could represent a proxy for ESC origin.

The perinatal and early childhood periods are times of dramatic transition in erythropoiesis and leukocyte function. Therefore, it was envisioned herein that this time of life would be marked by variations in embryonal to adult driven stem cell hematopoiesis. To test this idea, the relative proportion of cells with the FCO signature was examined in blood leukocytes from birth through old age (FIG. 14A). Table 7 shows data obtained for age specific ESC methylation fractions in blood leukocytes from birth (newborn) to old age (older than 65 years).

Dramatic and rapid decreases in the FCO cell fraction occurred over the first 5 years of life (FIG. 14A and FIG. 14B, and Table 7).

TABLE 7
Age specific estimated FCO methylation fractions in blood leukocytes from birth to old age

| Age group | | N | Min. | P10 | P25 | Median | Mean | SD | P75 | P90 | Max. | P |
|---|---|---|---|---|---|---|---|---|---|---|---|---|
| Newborn | | 60 | 67.5 | 74.4 | 78.5 | 82.3 | 82.0 | 6.0 | 85.6 | 88.8 | 97.6 | Reference |
| <12 | mo | 32 | 15.7 | 23.9 | 28.6 | 42.0 | 44.5 | 17.6 | 57.7 | 68.0 | 75.0 | 2.13 × 10⁻¹³⁴ |
| 12-18 | mo | 17 | 22.7 | 25.5 | 29.1 | 30.4 | 31.8 | 5.0 | 36.4 | 38.0 | 39.4 | 2.13 × 10⁻¹³⁴ |
| 18-24 | mo | 23 | 5.9 | 13.4 | 22.9 | 25.9 | 26.6 | 13.2 | 28.9 | 35.9 | 62.5 | 1.34 × 10⁻¹⁴⁷ |
| 2-5 | yr | 106 | 0 | 2.5 | 9.1 | 15.2 | 14.7 | 8.3 | 20.8 | 24.2 | 37.0 | 5.95 × 10⁻¹⁹⁸ |
| 5-18 | yr | 31 | 0 | 0 | 0 | 0.5 | 4.3 | 6.8 | 6.7 | 13.2 | 28.7 | <2.23 × 10⁻³⁰⁸ |
| 18-65 | yr | 403 | 0 | 0 | 0 | 0 | 3.1 | 4.5 | 5.6 | 9.43 | 26.5 | <2.23 × 10⁻³⁰⁸ |
| >65 | yr | 381 | 0 | 0 | 0 | 0 | 1.6 | 3.5 | 1.5 | 5.97 | 25.8 | <2.23 × 10⁻³⁰⁸ |

Notes:
Minimum, maximum, percentile cutoff values (10, 25, 50, 75, 90), mean and standard deviations derived from population data combined from published methylation datasets: see Supplemental Table 1. Values <0.1 were coded as 0. The reported P are based on linear model estimations adjusting for the age group using the newborns as the reference. We also used a linear mixed effect model adjusting for subject (for those measures with several samples), and Study as random effects, the P (using the Kenward Roger approximation for the degrees of freedom) were <2.23 × 10⁻³⁰⁸ for all the groups compared to the newborns.

A reduction in the proportion of cells with the FCO signature of approximately 60% was observed at 1.5 yrs. and by age 5 the fraction was reduced by 80%. Most adults (>18 yrs.) demonstrated non-detectable levels of cells with the FCO signature. However, approximately 10% of adults (18-65 yrs.), were observed to have a relatively high fraction of leukocytes with the FCO signature (range=10%-25%). The FCO fraction among adults with detectable FCO levels (more than 0%) showed a poor linear correlation (r=−0.12) with age. However, when restricting to those with FCO levels>3% and above, this correlation between FCO and age was no longer significant (r=−0.12, P>0.05). Of further note, there was no overlap in the loci comprising the FCO signature with the previously described CpGs used to calculate DNA methylation age (Lowe et al. 2016, herein). Although age associated in the early postnatal period, the FCO signature loci did not overlap with Horvath's age-related epigenetic clock and/or other epigenetic clocks (Lowe et al. 2016, herein). In addition none of the CpG loci identified during HSC aging in mice (Sun D et al. 2014. Cell Stem Cell 14: 673-88) overlap with the FCO signature used herein. These results indicate a distinction between aging and developmentally timed maturation events signaling variations in the fetal origin cell compartment (Rossi D J et al. 2008. Cell 132: 681-96).

Examples herein represent a conceptual departure from previous studies that have focused on DMRs that mark fate determination during terminal differentiation. Most of the characteristic DMRs of stem/progenitor cells are considered unstable to differentiation as they undergo transitions within the progeny as cells differentiate (Beerman I et al. 2013. Cell Stem Cell 12: 413-25; Farlik M et al. 2016. Cell Stem Cell 19: 808-822). In contrast, a smaller set of DMRs retain their status throughout the differentiation sequence and thus form a memory trace of cell origin. By restricting the initial CpG selection to lineage invariant loci, unstable loci (loci with additional sources of variability unrelated to the stem cell/progenitor origin) were filtered out. Subsetting invariant loci according to their differential methylation in newborn versus adult leukocytes was used to obtain an “orthogonal” set of developmentally sensitive loci.

The potential advantage of DNA methylation as a tracking strategy compared with previous methods (e.g. retroviral insertion, molecular barcodes) is that it relies on features of stem cells that have not been genetically altered. DNA methylation-based methods can be applied to human cells without manipulation, using fresh or archival specimens (such as those of ongoing birth cohorts), and provide a significant advantage in being a window into in vivo cell ontogeny dynamics. An example of the utility of this approach is evident in the study herein of newborns, infants and children that revealed a dramatic shift in hematopoietic ontogeny from birth to age 5 with evidence of wide individual variability. There is a great deal of interest in how the timing of early life developmental events shape life-long health outcomes (Gluckman P D et al. 2008. N Engl J Med 359: 61-73). The FCO provides an easily applied developmental marker of early immunologic maturation in such studies.

The loci represented in the FCO signature are themselves potential candidates with regulatory function in stem cell maturation. A notable example is the finding herein of DMRs in the Chromosome 22 region containing a cluster of let-7 microRNAs. Research has shown that expression of let-7 microRNAs play essential roles in the differentiation of embryonic stem cells (Lee H et al. 2016. Protein Cell 7: 100-113). The maintenance of the pluripotent state requires suppression of let-7. The DMR region we identified encompasses exon and intron 1 of MIRLET7BHG. Methylation in this region displayed a bipartite pattern and described an inverse relationship within fetal and adult cells wherein regulatory regions were hypermethylated in the fetal cells. This novel pattern was unexpected as hypermethylation in MIRLET7BHG has only been reported in infant leukemic cells (Nishi M et al. 2013. Leukemia 27: 389-97), wherein methylation silenced MIRLET7BHG expression. In contrast, the primary physiologic mechanism for let-7 regulation has been thought to involve post-transcriptional interference with microRNA biogenesis promoted through the actions of the L1N28A and LIN28B proteins (Lee et al. 2016, herein). LIN28A/LIN28B proteins are essential for normal development and contribute to the pluripotent state by preventing the maturation of let-7 pre-RNA (Piskounova E et al. 2008. J Biol Chem 283: 21310-4; Piskounova E et al. 2011. Cell 147: 1066-79). In turn, let-7 feeds back and dampens the expression of LIN28A/LIN28B thus forming a reciprocal negative feedback loop and acts as a bimodal switch (Rybak A et al. 2008. Nat Cell Biol 10: 987-993; Melton C et al. 2010. Nature 463: 621-6). Recent studies have identified novel DNA binding properties of Lin28 in mouse embryonic stem cells that may also modulate DNA methylation levels (Zeng Y et al. 2016. Mol Cell 61: 153-160). The data in examples herein are consistent with a DNA methylation mediated suppression of MIRLET7BHG in stem cells and its reversal via demethylation during the developmental switch leading to embryonic stem cell differentiation.

The selection herein of the candidates for the FCO signature took advantage of isolated subtypes of adult and newborn blood cells instead of using ESCs or hematopoietic progenitors. This approach was envisioned to be based on the requirement in the discovery step of making comparisons between homogeneous populations present in both newborns and adults and the fact that such data do not currently exist for the respective fetal and adult HSCs. Although an analysis using ESC and adult HSC was implemented, it was foreseen that the dynamic state within ESC subpopulations cannot correctly discriminate stochastic noise due to stem cell dynamics from the potential variation due to early cell commitment or coexistent cell states as observed in mouse models (Singer Z S et al. 2014. Mol Cell 55: 319-31). While starting with differentiated cells as in examples herein introduces some cell subpopulation heterogeneity (e.g. lymphocyte subpopulations) which cannot be controlled in our models, nonetheless, using UCB and AWB sorted blood samples allowed a clear contrast between the more general immune cell lineages in vivo. Under very controlled experimental conditions this same approach would have yielded a similar or an improved signature using ESC and a selected adult cell counterpart. Sensitivity analysis using ESC and adult CD34⁺ cells showed that at least 19% of the FCO signature was shared when using this approach.

The data also indicates that the method is a solution to a practical problem: when using ESC or FCO, the ex vivo conditions may generate heterogeneous populations of ESCs making them poor gold standards for comparison. In the absence of better standards, the proposed FCO signature provides a good proxy of the common fetal cell compartment. It is possible that the reduced FCO estimated fractions in higher passaged embryonic cells points to in vitro conditions leading to instability in the fetal epigenome and may constitute a quality control issue during the ex vivo manipulation of stem cells. The FCO fraction may provide one indicator of epigenome stability that could be useful in evaluating fetal cells expanded in vitro. An ongoing concern in adoptive cell transfer therapies is the paucity of informative markers reflecting epigenomic stability of expanded cell populations, as for example, in the expansion of umbilical cord blood derived

T-regulatory cells (Seay H R et al. 2017. Mol Ther Methods Clin Dev 4: 178-191).

Data herein have additional implications and potential applications for future applications. In clinical and epidemiological studies, the currently used cell correction methods (Titus A J et al. 2017. Hum Mol Genet 26: R216-R224; Teschendorff A E et al. 2017. BMC Bioinformatics 18: 105) could benefit from the additional information on cell heterogeneity provided by the FCO signature. As an adjunct to current cell correction methods the FCO can reduce variability in methylation signals due to cell composition and increase the specificity of EWAS analyses in identifying non-cell type causal factors. Large scale population studies must also account for the now well documented effects of age on a subset of DNA methylation loci, the so called Horvath clock CpG loci (Horvath 2013, herein), which are shown here to be distinct from those forming the FCO signature. Aging in humans is well known to alter hematopoiesis and recent studies in mice illustrate how it manifests in HSCs at multiple layers of the epigenome including DNA methylation (Sun D et al. 2014, herein). However, parallels of age-related HSC methylation with the FCO signature were not observed herein. None of the HSC age loci described in mice overlap with the FCO target loci. The phenomenon of clonal hematopoiesis of indeterminate potential (CHIP) is another age related hematopoietic variation of great potential clinical import (Jaiswal S et al. 2017. N Engl J Med 377: 1400-1402; Jaiswal S et al. 2014. N Engl J Med 371: 2488-98). It is known that CHIP occurs in about 10% of otherwise healthy persons of advanced age, which is similar to our FCO observations (Table 7). However, in examples herein with 784 different adult samples (>18 yrs) no significant correlation of the FCO was observed with the age of blood donors. In the absence of an age-related explanation for increased FCO fractions in some adults is a heretofore unrecognized cell component in adult blood having a distinct fetal cell ontogeny is hypothesized.

In this regard the FCO provides a tool to help resolve a long-debated controversy about the occurrence of a B1 subtype of B-lymphocytes in humans (Griffin D O et al. 2011. J Exp Med 208: 2566-9; Descatoire M et al. 2011. J Exp Med 208: 2563-4; Hardy R R et al. 2015. Eur J Immunol 45: 2978-84). In mice, B1 cells are well described as long-lived self-renewing fetal derived B-cells that produce natural antibodies in the absence of apparent antigenic stimulation and localize in pleural and peritoneal cavities in adults (Hardy et al. 2015, herein; Ghosn E E B et al. 2015. Ann N Y Acad Sci 1362: 23-38). Furthermore, an important role has been established for Let-7 microRNA in mouse B1 cell development (Yuan J et al. 2012. Science 335: 1195-1200), and data herein have linked differential methylation of MIRLET7BHG with the human fetal signature. To explore the hypothesis that the blood FCO signal can arise from a unique B cell population will require isolation of candidate B1 cell populations and simultaneous measurement of the FCO fraction. Human resident macrophages are another potential fetal derived cell type in adult tissues (Hoeffel G et al. 2018. Cell Immunol 1-40; Hoeffel G et al. 2015. Front Immunol 6: 486); the FCO signature could provide a means to explore epigenetic features of the ontogeny of these cells as well.

Finally, a surprising observation herein was made that non-hematopoietic tissues also demonstrate a marked developmental age variation in the FCO signature fraction in fetal tissues. There was evidence of heterogeneity in the FCO signature fraction in brain and muscle according to fetal gestational age. This observation, which is consistent with previous studies in fetal brain (Jaffe A E et al. 2016. Nat Neurosci 19: 40-7) indicates that the transition observed postnatally in hematopoietic cells occurs prenatally in a tissue dependent fashion. Therefore, the FCO signature may be a tool that is useful to explore stem cell heterogeneity more broadly in human development. In conclusion, a DNA methylation signature is provided herein which is common among human fetal hematopoietic progenitor cells, and it is shown that this signature traces the lineage of cells and informs the study of stem cell heterogeneity in humans under homeostatic conditions.

What is claimed is:

1. A method for obtaining a stem cell DNA methylation signature in a subject, comprising:
identifying subsets of methylation invariant CpGs within nucleotide sequences of a plurality of leukocyte subtypes in a prenatal or neonatal sample and in an adult sample, and selecting a subset of identified CpGs containing differentially methylated regions (DMRs) between prenatal or neonate leukocyte subtypes and adult leukocyte subtypes;
determining CpGs within a resulting selected subset that are variant between the samples, and determining CpGs within the same selected subset that are invariant between leukocyte subtypes, and comparing the determined variant CpGs and the determined invariant CpGs, to select the leukocyte subtype invariant CpGs for inclusion in a subset list; and,
preparing a stem cell methylation signature by statistically removing CpGs from the subset list based on inconsistent coefficient sign in model estimates delta beta coefficient models, and selecting the leukocyte subtype invariant CpGs with a statistical difference in methylation between the adult and prenatal or neonate samples which is greater than a pre-determined threshold, to obtain the stem cell methylation signature.

2. The method according to claim 1, wherein preparing further comprises deconvoluting a prenatal sample methylation fraction or neonate sample methylation fraction compared to all adult sample methylation fraction using constrained projection quadratic programming (CP/QP), the stem cell methylation signature being substituted for a default reference methylation library.

3. The method according to claim 1, wherein the stem cell methylation signature is enriched by applying a hypergeometric test to the stem cell methylation signature that reduces the stem cell methylation signature to CpG sequences providing maximum differences in methylation status between the prenatal or neonate sample and the adult sample by a confirmatory principal component analysis with a first component and at least one second component.

4. The method according to claim 3, wherein the first component determines the CpGs that are variant in methylation status between the prenatal sample or the neonate sample and the adult sample by using a pairwise linear model and second components determine the CpGs that are invariant in methylation status among leukocyte subtypes using a linear mixed effect model adjusted using limma to account for subject differences.

5. The method according to claim 4, further comprising calculating the geometric angle between the first component and the second components.

6. The method according to claim 5, further comprising selecting CpGs with maximum orthogonality of the calculated geometric angle for inclusion in the stem cell methylation signature.

7. The method according to claim 1, wherein constrained projection quadratic programming (CP/QP) is calculated according to the equation: arg min_{w}∥Y−wM^{T}∥², wherein M is the list of CpGs, w is an estimate of a fraction of cells carrying the stem cell lineage signature, and Y is based on the constrained projection quadratic programming (CP/QP).

8. The method according to claim 1, further comprising validating the stem cell signature by geometrically comparing DNA methylation profiles of purified leukocyte cell subtypes, by obtaining the profiles from at least one methylation library, to DNA methylation profiles of the stem cell methylation signature.

9. The method according to claim 1, further comprising validating the stem cell signature by geometrically comparing DNA methylation profiles of synthetic cell mixtures containing known proportions of the prenatal sample or the neonate sample and the adult sample to a DNA methylation profile of the stem cell methylation signature.

10. The method according to claim 1, further comprising pooling the methylation datasets of the at least one prenatal or neonatal sample and the at least one adult sample to combine at least one methylation data subset for a specified subset of leukocyte subtypes.

11. The method according to claim 1, further comprising adjusting mathematically the methylation datasets of the at least one prenatal sample or neonate sample and the at least one adult sample to account for at least one variable of the subject from which the samples were obtained, the variables selected from the group of: sex, DNA methylation age, and subject indicators.

12. The method according to claim 1, further comprising implementing by the hypergeometric test the methylation reference databases to restrict the background to genes interrogated in a methylation array, and applying statistical methods to the methylation data to account for array bias.

13. The method according to claim 3, further comprising using the confirmatory principal component analysis first component to account for differences in the adult sample compared to the prenatal or the neonate sample, and the second component to account for subject variability and residual cell subtype confounding.

14. The method according to claim 1, wherein the stem cell methylation signature includes a plurality of sequences selected from the group of: cg10338787 (SEQ ID No: 68), cg22497969 (SEQ ID No: 83), cg11968804 (SEQ ID No: 71), cg10237252 (SEQ ID No: 67), cg17310258 (SEQ ID No: 78), cg13485366 (SEQ ID No: 72), cg03455765 (SEQ ID No: 62), cg04193160 (SEQ ID No: 63), cg27367526 (SEQ ID No: 85), cg03384000 (SEQ ID No: 61), cg15575683 (SEQ ID No: 76), cg17471939 (SEQ ID No: 79), cg11199014 (SEQ ID No: 70), cg13948430 (SEQ ID No: 73), cg01567783 (SEQ ID No: 59), cg01278041 (SEQ ID No: 60), cg19005955 (SEQ ID No: 80), cg16154155 (SEQ ID No: 77), cg14652587 (SEQ ID No: 75), cg19659741 (SEQ ID No: 81), cg06705930 (SEQ ID No: 65), cg23009780 (SEQ ID No: 84), cg22130008 (SEQ ID No: 82), cg05840541 (SEQ ID No: 64), cg06953130 (SEQ ID No: 66), cg11194994 (SEQ ID No: 69), and cg14375747 (SEQ ID No: 74).

15. The method according to claim 1, wherein the prenatal or neonatal sample is a cell or a tissue obtained from at least one of the group consisting of: a fetus, an umbilical cord, umbilical blood, an infant, a uterus, a vein, an artery, a tumor, an abnormal growth, bone marrow, a transplanted or a re-sectioned biological material, an embryo, and a cell from an embryo.

16. Uses of the methods herein for selecting a small number of nucleotide sequences for a custom array for efficient and economical determination of at least one of embryonic cell content, stem cell content, experiential exposure on stem cell maturation, and identity of progenitor cell lineages.

17. A method for determining effects of experiential exposure on stem cell maturation in a subject, comprising:
obtaining an exposure sample and a control sample from the subject and analyzing extent of methylation of at least one CpG dinucleotide in DNA of each sample within a plurality of oligonucleotides sequences selected from at least one of the group of cg10338787 (SEQ ID No: 68), cg22497969 (SEQ ID No: 83), cg11968804 (SEQ ID No: 71), cg10237252 (SEQ ID No: 67), cg17310258 (SEQ ID No: 78), cg13485366 (SEQ ID No: 72), cg03455765 (SEQ ID No: 62), cg04193160 (SEQ ID No: 63), cg27367526 (SEQ ID No: 85), cg03384000 (SEQ ID No: 61), cg15575683 (SEQ ID No: 76), cg17471939 (SEQ ID No: 79), cg11199014 (SEQ ID No: 70), cg13948430 (SEQ ID No: 73), cg01567783 (SEQ ID No: 60), cg01278041 (SEQ ID No: 59), cg19005955 (SEQ ID No: 80), cg16154155 (SEQ ID No: 77), cg14652587 (SEQ ID No: 75), cg19659741 (SEQ ID No: 81), cg06705930 (SEQ ID No: 65), cg23009780 (SEQ ID No: 84), cg22130008 (SEQ ID No: 82), cg05840541 (SEQ ID No: 64), cg06953130 (SEQ ID No: 66), cg11194994 (SEQ ID No: 69), and cg14375747 (SEQ ID No: 74), thereby determining a methylation status of at least one CpG dinucleotide in the DNA of the exposure sample and a methylation status of at least one CpG dinucleotide in the DNA of the control sample; and,
deconvoluting the methylation array data from the control sample and the exposure sample to obtain methylation status of individual leukocyte subtypes in the samples, and comparing methylation status of the at least one CpG dinucleotide within a leukocyte subtype of the control sample to the methylation status of the at least one CpG dinucleotide within the same leukocyte subtype of the exposure sample, to determine sites of differential methylation, and correlating a difference in methylation status between the control sample and the exposure sample to obtain the effect of the exposure on stem cell methylation signature.

18. The method according to claim 17, wherein correlating further comprises assessing the effects of at least one of the following on the stem cell methylation signature: a therapy, a vaccine, a nutritional regimen, a genetic alteration, a progenitor cell transplant, and an environmental exposure.

19. The method according to claim 17, wherein correlating further comprises diagnosing prenatal abnormalities in a fetus.

20. The method according to claim 17, wherein correlating further comprises altering patient therapies through analysis of stem cell methylation in induced pluripotent stem cells therapies in the subject.

21. The method according to claim 17, wherein correlating further comprises determining amount of induction of stem cell progenitors in a transplantation procedure.

22. The method according to claim 17, wherein correlating further comprises measuring an extent of reprogramming adult cells into induced pluripotent stem cells, thereby obtaining a quality control parameter.

23. A kit for determining embryonic stem cell methylation signatures, comprising:
an array with a plurality of DNA probes attached to a surface or a plurality of surfaces at known addressable locations on the array, wherein the probes hybridize to a DNA sequence of each of a methylated form and an unmethylated form of a CpG dinucleotide in a sequence of a gene of the plurality of genes in the sample;
primers and reagents for detecting the hybridized probes and for detecting the reaction products derived from the hybridized probes to obtain methylation data; and
instructions for analyzing at least one sample on the array, and instructions for preparing a stem cell methylation signature.

24. A method for identifying progenitor cell lineages, comprising:
comparing DNA methylation profiles of a leukocyte subtype between a prenatal or neonatal sample and an adult sample;
identifying CpG sites differentially methylated between the prenatal or neonatal sample and the adult sample for the leukocyte subtype;
filtering to select a lineage invariant subset of CpG loci, the subset loci having consistent differential methylation between the leukocyte subtype and an absolute change in methylation greater than a pre-determined threshold between the prenatal or neonatal sample and the adult sample, thereby forming a candidate list of CpG loci for a stem cell methylation signature; and
reducing the candidate list of CpG loci for the stem cell methylation signature by selecting CpGs with minimal residual cell-specific effects, thereby forming a block of differentially methylated regions (DMRs) across the progenitor cell axis of multipotency to terminal differentiation, to identify the progenitor cell lineages.

25. The method according to claim 24, further comprising: calculating a leukocyte proportion exhibiting the stem cell methylation signature, by applying constrained projection quadratic programming (CP/QP) to the candidate list of the stem cell methylation signature CpG loci.

26. The method according to claim 25, wherein calculating further comprises iterating with at least one additional set of leukocyte sequences from each of the prenatal or neonatal sample and the adult sample sources to confirm the candidate list of the CpG loci for the stem cell methylation signature as an estimator of the fraction of the leukocytes in a mixture that contains lineage invariant and developmentally sensitive stem cell loci.

27. The method according to claim 26, further comprising:
validating the calculated stem cell methylation signatures by preparing mixtures of the prenatal or neonate sample and the adult sample in known relative amounts, thereby generating synthetic cell mixtures;
analyzing the synthetic cell mixtures on a DNA methylation array to determine methylation status of CpG dinucleotides in the leukocytes in the mixtures; and
applying statistical methods to the obtained methylation array data of the mixtures to correlate the fraction of cells carrying a stem cell methylation signature with the known mixture relative amounts, thereby determining stem cell maturation by the changes in methylation status between the prenatal or neonate sample leukocytes and the adult sample leukocytes.

28. A method of using an array to determine an embryonic stem cell (ESC) methylation signature in a biological sample, comprising:
analyzing extent of DNA hybridization in an adult sample and a prenatal or neonatal sample to each of a plurality of oligonucleotide probes, the probes being affixed to at least a first surface for methylated CPG sequences and a second surface for unmethylated CpG sequences, the DNA sequences of the oligonucleotides on the first surface and the second surface being otherwise identical, the plurality of the nucleotide sequences selected from at least one of the group of cg10338787 (SEQ ID No: 68), cg22497969 (SEQ ID No: 83), cg11968804 (SEQ ID No: 71), cg10237252 (SEQ ID No: 67), cg17310258 (SEQ ID No: 78), cg13485366 (SEQ ID No: 72), cg03455765 (SEQ ID No: 62), cg04193160 (SEQ ID No: 63), cg27367526 (SEQ ID No: 85), cg03384000 (SEQ ID No: 61), cg15575683 (SEQ ID No: 76), cg17471939 (SEQ ID No: 79), cg11199014 (SEQ ID No: 70), cg13948430 (SEQ ID No: 73), cg01567783 (SEQ ID No: 60), cg01278041 (SEQ ID No: 59), cg19005955 (SEQ ID No: 80), cg16154155 (SEQ ID No: 77), cg14652587 (SEQ ID No: 75), cg19659741 (SEQ ID No: 81), cg06705930 (SEQ ID No: 65), cg23009780 (SEQ ID No: 84), cg22130008 (SEQ ID No: 82), cg05840541 (SEQ ID No: 64), cg06953130 (SEQ ID No: 66), cg11194994 (SEQ ID No: 69), and cg14375747 (SEQ ID No: 74), for determining methylation status of at least one CpG dinucleotide in the DNA of each of the adult and the prenatal or neonatal sample sample;
deconvoluting the methylation array data from the adult sample and the prenatal or neonatal sample to obtain methylation data of a plurality of leukocyte subtypes in the samples;
comparing methylation status of the at least one CpG dinucleotide for a leukocyte subtype in the adult sample to the methylation status of the at least one CpG dinucleotide of the leukocyte subtype of the prenatal or neonatal sample, to determine differentially methylated regions (DMRs); and
analyzing the DMRs to determine the fraction of sequences from progenitor cell lineage origin which constitutes the ESC methylation signature.

29. The method according to claim 28, further comprising comparing the ESC methylation signature of samples of a first subject and a second subject, wherein the first and second subjects are assessed for effects on the embryonic stem cell methylation signature of differences in maternal or prenatal conditions selected from the group of: nutrition, nutrition, genetics, infant or embryonic genetics, environmental exposure, hematopoietic stress, treatment with chemical agents, vaccination status, transplantation, and surgical stress.

30. The method according to claim 28, further comprising comparing the ESC methylation signature during cancer therapy induced neutropenia in a sample from a patient being treated with an agent that promote granulopoiesis, with the ESC methylation signature obtained prior to treatment.

31. The method of claim 28, further comprising inducing CD34 stem progenitors for transplantation, and comparing effect on the ESC methylation signatures to determine quality of the induction process.

32. The method according to claim 14 or 23, wherein each of the plurality of sequences comprises a portion of cg10338787 (SEQ ID No: 68), cg22497969 (SEQ ID No: 83), cg11968804 (SEQ ID No: 71), cg10237252 (SEQ ID No: 67), cg17310258 (SEQ ID No: 78), cg13485366 (SEQ ID No: 72), cg03455765 (SEQ ID No: 62), cg04193160 (SEQ ID No: 63), cg27367526 (SEQ ID No: 85), cg03384000 (SEQ ID No: 61), cg15575683 (SEQ ID No: 76), cg17471939 (SEQ ID No: 79), cg11199014 (SEQ ID No: 70), cg13948430 (SEQ ID No: 73), cg01567783 (SEQ ID No: 60), cg01278041 (SEQ ID No: 59), cg19005955 (SEQ ID No: 80), cg16154155 (SEQ ID No: 77), cg14652587 (SEQ ID No: 75), cg19659741 (SEQ ID No: 81), cg06705930 (SEQ ID No: 65), cg23009780 (SEQ ID No: 84), cg22130008 (SEQ ID No: 82), cg05840541 (SEQ ID No: 64), cg06953130 (SEQ ID No: 66), cg11194994 (SEQ ID No: 69), and cg14375747 (SEQ ID No: 74).

33. The method according to claim 32, wherein the portion includes at least one hypermethylatable CpG.

34. The method according to claim 17, wherein extent of methylation is determined by hybridizing each DNA sample to each of a plurality of oligonucleotide probes attached to at least one array, the probes affixed to at least one surface and containing each of methylated CpG containing oligonucleotide sequences and unmethylated CpG containing oligonucleotide sequences and otherwise identical in nucleotide sequence.

35. The method according to claim 17, wherein extent of methylation is determined by amplifying sample DNA by polymerase chain reaction (PCR) with primers specific for hypermethylated Cpg dinucleotides.

36. An array for efficient and economical determination of embryonic stem cell (ESC) content in a biological sample, comprising a surface containing a plurality of nucleotide sequences, each sequence at an addressable location, the sequences selected from at least one of the group of: cg10338787 (SEQ ID No: 68), cg22497969 (SEQ ID No: 83), cg11968804 (SEQ ID No: 71), cg10237252 (SEQ ID No: 67), cg17310258 (SEQ ID No: 78), cg13485366 (SEQ ID No: 72), cg03455765 (SEQ ID No: 62), cg04193160 (SEQ ID No: 63), cg27367526 (SEQ ID No: 85), cg03384000 (SEQ ID No: 61), cg15575683 (SEQ ID No: 76), cg17471939 (SEQ ID No: 79), cg11199014 (SEQ ID No: 70), cg13948430 (SEQ ID No: 73), cg01567783 (SEQ ID No: 60), cg01278041 (SEQ ID No: 59), cg19005955 (SEQ ID No: 80), cg16154155 (SEQ ID No: 77), cg14652587 (SEQ ID No: 75), cg19659741 (SEQ ID No: 81), cg06705930 (SEQ ID No: 65), cg23009780 (SEQ ID No: 84), cg22130008 (SEQ ID No: 82), cg05840541 (SEQ ID No: 64), cg06953130 (SEQ ID No: 66), cg11194994 (SEQ ID No: 69), and cg14375747 (SEQ ID No: 74), for analyzing a fraction of sequences of progenitor cell lineage origin having an ESC methylation signature.

37. The array according to claim 36, wherein the array is efficient and economical for determination of the content, comprising nucleotide sequences containing CpG sites which are less than 1%, less than 0.1%, 0.01% or 0.001% of total CpG sequences in a genome.

38. An array having the uses of determining any of embryonic cell content, stem cell content, experiential exposure on stem cell maturation, and identity of progenitor cell lineages having nucleotide sequences containing at least one CpG selected by any of the methods herein from among 25 million CpGs in the human genome.

39. A kit for determining embryonic cell content, the kit comprising a plurality of primers for custom bisulfate sequencing library preparation, each primer directing amplification of a hyper methylatable CpG dinucleotide located in a DNA sequence selected from cg10338787 (SEQ ID No: 68), cg22497969 (SEQ ID No: 83), cg11968804 (SEQ ID No: 71), cg10237252 (SEQ ID No: 67), cg17310258 (SEQ ID No: 78), cg13485366 (SEQ ID No: 72), cg03455765 (SEQ ID No: 62), cg04193160 (SEQ ID No: 63), cg27367526 (SEQ ID No: 85), cg03384000 (SEQ ID No: 61), cg15575683 (SEQ ID No: 76), cg17471939 (SEQ ID No: 79), cg11199014 (SEQ ID No: 70), cg13948430 (SEQ ID No: 73), cg01567783 (SEQ ID No: 60), cg01278041 (SEQ ID No: 59), cg19005955 (SEQ ID No: 80), cg16154155 (SEQ ID No: 77), cg14652587 (SEQ ID No: 75), cg19659741 (SEQ ID No: 81), cg06705930 (SEQ ID No: 65), cg23009780 (SEQ ID No: 84), cg22130008 (SEQ ID No: 82), cg05840541 (SEQ ID No: 64), cg06953130 (SEQ ID No: 66), cg11194994 (SEQ ID No: 69), and cg14375747 (SEQ ID No: 74).

40. A kit for determining embryonic stem cell methylation signatures, comprising:
an array with a plurality of DNA probes attached to a surface or a plurality of surfaces at known addressable locations on the array, wherein the probes hybridize to a DNA sequence of each of a methylated form and an unmethylated form of a CpG dinucleotide in a sequence of a gene of the plurality of genes in the sample or; a set of oligonucleotide primers comprising a plurality of sequences each having a CpG dinucleotide within each primer sequence;
primers and reagents for detecting the hybridized probes and for detecting the reaction products derived from the hybridized probes to obtain methylation data; and
instructions for analyzing at least one sample on the array, and instructions for preparing a stem cell methylation signature.

41. A kit for quantifying embryonic stem cells in a biological sample, the kit comprising: at least one of
(i) an array with a plurality of DNA probes attached to a surface or a plurality of surfaces at known addressable locations on the array, wherein the probes hybridize to a DNA sequence of each of a methylated form and an unmethylated form of a CpG dinucleotide in a stem cell signature sequence in the sample; and/or
(ii) a plurality of oligonucleotide primers comprising a plurality of gene sequences in the stem cell signature for amplification of genomic DNA at a plurality of loci corresponding to hypermethylated CpG sites; and
reagents comprising at least one of: primers for amplifying DNA in the sample, for detecting sample DNA hybridized with probes, and for detecting reaction products derived from the hybridized probes to obtain methylation data; and
instructions for analyzing at least one sample on the array, and instructions for quantifying embryonic stem cells based on the stem cell methylation signature.

42. Uses of a list of 27 CpG containing loci in the human genome as a stem cell methylation signature for efficient and economical determination of at least one of embryonic cell content, stem cell content, experiential exposure on stem cell maturation, and identity of progenitor cell lineages.

43. A method for quantifying effects of experiential exposure on stem cell maturation in a subject, comprising:
obtaining an exposure sample and a control sample from the subject and analyzing extent of methylation of at least one CpG dinucleotide in DNA of each sample within a plurality of CpG dinucleotide locations selected from at least one of the group of cg10338787, cg22497969, cg11968804, cg10237252, cg17310258, cg13485366, cg03455765, cg04193160, cg27367526, cg03384000, cg15575683, cg17471939, cg11199014, cg13948430, cg01567783, cg01278041, cg19005955, cg16154155, cg14652587, cg19659741, cg06705930, cg23009780, cg22130008, cg05840541, cg06953130, cg11194994, and cg14375747, thereby determining a methylation status of at least one CpG dinucleotide in the DNA of the exposure sample and a methylation status of at least one CpG dinucleotide in the DNA of the control sample; and,
deconvoluting the methylation array data from the control sample and the exposure sample to obtain methylation status of individual leukocyte subtypes in the samples, and comparing methylation status of the at least one CpG dinucleotide within a leukocyte subtype of the control sample to the methylation status of the at least one CpG dinucleotide within the same leukocyte subtype of the exposure sample, to determine sites of differential methylation, and correlating a difference in methylation status between the control sample and the exposure sample to obtain the effect of the exposure on stem cell methylation signature.

44. A kit for quantifying embryonic cell from extent of hypermethylation, the kit comprising a plurality of primers for custom bisulfate sequencing library preparation, each primer directing amplification of a hyper methylatable CpG dinucleotide located in a DNA sequence selected from cg10338787, cg22497969, cg11968804, cg10237252, cg17310258, cg13485366, cg03455765, cg04193160, cg27367526, cg03384000, cg15575683, cg17471939, cg11199014, cg13948430, cg01567783, cg01278041, cg19005955, cg16154155, cg14652587, cg19659741, cg06705930, cg23009780, cg22130008, cg05840541, cg06953130, cg11194994, and cg14375747.

45. An array for quantifying embryonic stem cell (ESC) content in a biological sample, comprising a surface containing a plurality of hypermethylatable CpG locations, the locations selected from at least one of the group of: cg10338787, cg22497969, cg11968804, cg10237252, cg17310258, cg13485366, cg03455765, cg04193160, cg27367526, cg03384000, cg15575683, cg17471939, cg11199014, cg13948430, cg01567783, cg01278041, cg19005955, cg16154155, cg14652587, cg19659741, cg06705930, cg23009780, cg22130008, cg05840541, cg06953130, cg11194994, and cg14375747, for analyzing ESC content having an ESC methylation signature.
